(19) United States
(12) Patent Application Publication
(10) Pub. No.: US 2022/0105108 A1
(43) Pub. Date: Apr. 7, 2022

(54) PERIVASCULAR ANTI-INFLAMMATORY THERAPY FOR VENOUS THROMBOSIS

(71) Applicant: Mercator MedSystems, Inc., Emeryville, CA (US)

(72) Inventor: Kirk P. SEWARD, Brooklyn, NY (US)

(21) Appl. No.: 17/491,036

(22) Filed: Sep. 30, 2021

Related U.S. Application Data
(60) Provisional application No. 63/086,228, filed on Oct. 1, 2020.

(51) Int. Cl. A61K 31/573; A61K 9/00; A61P 7/04

(52) U.S. Cl. CPC A61K 31/573; A61P 7/04; A61K 9/0024

(57) ABSTRACT

Disclosed herein are methods, devices, systems, and kits for reducing inflammation and rate of progression to post-thrombotic syndrome (PTS) in individuals who have experienced venous thrombosis. Provided herein are approaches for local delivery of therapeutic agents to reduce inflammation and resolve clotting in affected veins in limbs. A catheter is positioned within the affected vein, and a composition comprising one or more therapeutic agents is injected into the perivenous tissue through the wall of the vein. The puncturing to inject may be achieved by an expanding balloon on the distal end of the catheter.

### CROSS-REFERENCE

This application claims the benefit of U.S. Provisional Application No. 63/086,228 filed Oct. 1, 2020, which is incorporated herein by reference.

The subject matter of the present application is related to the subject matter of U.S. application Ser. No. 16/977,355, filed on Sep. 1, 2020, which is a national phase entry of International Application No. PCT/US19/22054, filed on Mar. 13, 2019, which claims priority from U.S. Provisional Application No. 62/642,743, filed on Mar. 14, 2018, which are incorporated herein by reference

### BACKGROUND

Individuals having deep vein thrombosis (DVT) or blood clots in blood vessels may experience post-thrombotic syndrome (PTS). PTS has been associated with local venous inflammation and changes in levels of inflammatory factors. Individuals with DVT may experience PTS even after compression therapy, pharmaceutical treatments, or thrombolysis or interventional or open surgical procedures to treat the DVT. Symptoms of PTS may include sensations of leg heaviness, pulling, or fatigue, leg pain, and limb swelling. As such, a local delivery of agents that target the inflammatory response may reduce the symptoms of PTS and provide a useful treatment for PTS.

### SUMMARY

Disclosed herein are device, methods, and kits for treatment of post-thrombotic syndrome (PTS) in an individual. Provided herein are device, methods, and kits for treatment of symptoms from resulting from deep vein thrombosis (DVT) or blood clots in blood vessels in an individual. Described herein are device, methods, and kits to reduce or resolve inflammation that is present with PTS and/or venous thromboembolism, including but not limited to DVT and pulmonary embolism (PE).

Provided herein are methods of reducing progression to post-thrombotic syndrome (PTS) in a subject, the method comprising: (a) identifying a vein in the subject affected by deep vein thrombosis (DVT) currently or previously and/or is at risk for progressing to PTS; (b) advancing a therapeutic delivering catheter within a lumen of the vein affected by DVT to or near a thrombosed segment of the vein; and (c) delivering a therapeutic composition into a perivascular tissue at or near the thrombosed segment using the therapeutic delivering catheter, wherein the therapeutic composition comprises an anti-inflammatory agent and a therapeutic dosage of the anti-inflammatory agent ranges from about 0.1 mg per cm of the thrombosed segment to about 10 mg per cm of the thrombosed segment. In some embodiments, the anti-inflammatory agent comprises a glucocorticoid. In some embodiments, the glucocorticoid comprises dexamethasone. In some embodiments, the vein affected by DVT comprises a plurality of thrombotic segments. In some embodiments, the therapeutic composition is delivered to the plurality of thrombosed segments. In some embodiments, the vein affected by DVT has undergone a catheter-directed thrombolysis or thrombectomy (CDT) previously. In some embodiments, the vein affected by DVT has undergone an endovascular procedure previously, wherein the endovascular procedures comprise one or more of venous valve repair, venous bypass, and venous stents. In some embodiments, a total dosage of the anti-inflammatory agent delivered into the vein affected by DVT ranges between about 1 mg and about 100 mg. In some embodiments, a therapeutic concentration of the anti-inflammatory agent delivered into the vein affected by DVT ranges between about 0.1 mg/ml to about 10 mg/ml. In some embodiments, a volume of the anti-inflammatory agent delivered into the vein affected by DVT ranges between about 0.01 ml per cm of the thrombosed vein to about 100 ml per cm of the thrombosed vein. In some embodiments, the therapeutic composition further comprises a fibrinolytic agent. In some embodiments, the fibrinolytic agent comprises one or more of tissue plasminogen activator (tPA), von Willebrand factor (vWF) inhibitor, G-CSF, P-selectin inhibitor, E-selection inhibitors, resolvins, protectins, MMP-9 inhibitors, low molecular weight heparin, tenecteplase, reteplase, alteplase, streptokinase and urokinase. In some embodiments, the fibrinolytic agent comprises tissue plasminogen activator (tPA). In some embodiments, the fibrinolytic agent is delivered directly into an acute or organizing thrombus. In some embodiments, the delivery of the fibrinolytic agent results in a resolution of a thrombus in the thrombosed segment. In some embodiments, the resolution of the thrombus takes at least 1 day, 3 days, 7 days, or 14 days. In some embodiments, the delivery of the fibrinolytic agent results in a maintenance or an increase in patency of the thrombosed segment. In some embodiments, the maintenance or the increase in patency lasts for at least 5 weeks, 3 months, 6 months, 12 months, 18 months, or 24 months. In some embodiments, a level of one or more inflammatory biomarkers decreases after the delivery of a therapeutic composition into a perivascular tissue at or near the thrombosed segment. In some embodiments, the one or more inflammatory biomarkers comprises one or more of IL-1β, IL-2, IL-6, IL-8, IL-10, IFN-α, IFN-γ, ICAM-1, TNF-α, CRP, D-dimer, fibrinogen, MCP-1, IL-1Ra, IL-1α, MMP-1, MMP-2, MMP-8, MMP-9, TIMP, ICAM-1, VCAM-1, and soluble P-selectin. In some embodiments, the level of one or more inflammatory biomarkers is measured from a sample from whole blood, plasma, serum, or perivascular tissue. In some embodiments, a level of one or more anti-inflammatory biomarkers increases after the delivery of a therapeutic composition into a perivascular tissue at or near the thrombosed segment. In some embodiments, the one or more anti-inflammatory biomarkers comprises one or more of IL-10 and IL-1 receptor antagonist (IL-1 Ra). In some embodiments, the reduction in progression to PTS is assessed by maintenance or an increase in patency of the thrombosed segment. In some embodiments, the maintenance or the increase in patency lasts for at least 5 weeks, 3 months, 6 months, 12 months, 18 months, or 24 months. In some embodiments, the reduction in progression to PTS is assessed by a decrease or a lack of increase in rethrombosis in the thrombosed segment. In some embodiments, the decrease or the lack of increase in rethrombosis lasts for at least 5 weeks, 3 months, 6 months, 12 months, 18 months, or 24 months. In some embodiments, the decrease or the lack of increase in rethrombosis is measured by ultrasound. In some embodiments, the reduction in progression to PTS is assessed by a decrease or a lack of increase in venous reflux. In some embodiments, the decrease or the lack of increase in venous reflux lasts for at least 5 weeks, 3 months, 6 months, 12 months, 18 months, or 24 months. In some embodiments, the decrease or the lack of increase in venous reflux is measured by ultrasound. In some embodiments, the reduction in progression to PTS is assessed by a decrease or a lack of increase in fibrosis and stiffness of wall and valve of the vein affected by DVT. In some embodiments, the decrease or the lack of increase in fibrosis and stiffness of wall and valve is measured by ultrasound. In some embodiments, the reduction in progression to PTS is assessed by a decrease or a lack of increase in a symptom of PTS, wherein the symptom of PTS comprises one or more of pain, cramps, heaviness, pruritus, paresthesia, edema, skin induration, hyperpigmentation, venous ectasia, redness, and pain during calf compression. In some embodiments, the reduction in progression to PTS is assessed by a decrease or a lack of increase in a Villalta score or a VCSS score. In some embodiments, the vein affected by DVT currently or previously and/or is at risk for progressing to PTS is identified by fluordeoxyglucose-positron emission tomography (FDG-PET). In some embodiments, the reduction in progression to PTS is assessed by FDG-PET scanning of the perivascular tissue. In some embodiments, the FDG-PET detects a level of local metabolic activity in the perivascular tissue. In some embodiments, the level of local metabolic activity indicates localized inflammation. In some embodiments, an increase in a residual local metabolic activity detected by FDG-PET indicates progression to PTS. In some embodiments, a decrease in a residual local metabolic activity detected by FDG-PET indicates reduction in progression to PTS. In some embodiments, the therapeutic composition comprises one or more component for extended release, sustained release, or controlled release. In some embodiments, the therapeutic composition is extended released, sustained released, or controlled released in the perivascular tissue. In some embodiments, the therapeutic delivering catheter accesses the vein affected by DVT from a popliteal vein. In some embodiments, the therapeutic delivering catheter comprises a needle injection catheter.

Described herein are methods of reducing progression to post-thrombotic syndrome (PTS) in a subject, the method comprising: (a) identifying a vein in the subject affected by deep vein thrombosis (DVT) currently or previously; (b) advancing a therapeutic delivering catheter within a lumen of the vein affected by DVT to or near a thrombosed segment of the vein; and (c) delivering a therapeutic composition into a perivascular tissue at or near the thrombosed segment using the therapeutic delivering catheter, wherein the therapeutic composition comprises mononuclear stem or stem-like cells. In some embodiments, the vein affected by DVT comprises a plurality of thrombotic segments. In some embodiments, the therapeutic composition is delivered to the plurality of thrombosed segments. In some embodiments, the vein affected by DVT has undergone a catheter-directed thrombolysis or thrombectomy (CDT) previously. In some embodiments, a level of one or more inflammatory biomarkers decreases after the delivery of a therapeutic composition into a perivascular tissue at or near the thrombosed segment. In some embodiments, the one or more inflammatory biomarkers comprises one or more of IL-1β, IL-2, IL-6, IL-8, IL-10, IFN-α, IFN-γ, ICAM-1, TNF-α, CRP, D-dimer, fibrinogen, MCP-1, IL-1Ra, IL-1α, MMP-1, MMP-2, MMP-8, MMP-9, TIMP, ICAM-1, VCAM-1, and soluble P-selectin. In some embodiments, the level of one or more inflammatory biomarkers is measured from a sample from whole blood, plasma, serum, or perivascular tissue. In some embodiments, the reduction in progression to PTS is assessed by a decrease or a lack of increase in a symptom of PTS, wherein the symptom of PTS comprises one or more of pain, cramps, heaviness, pruritus, paresthesia, edema, skin induration, hyperpigmentation, venous ectasia, redness, and pain during calf compression. In some embodiments, the reduction in progression to PTS is assessed by a decrease or a lack of increase in venous reflux. In some embodiments, the decrease or the lack of increase in venous reflux lasts for at least 5 weeks, 3 months, 6 months, 12 months, 18 months, or 24 months. In some embodiments, the decrease or the lack of increase in venous reflux is measured by ultrasound. In some embodiments, the reduction in progression to PTS is assessed by a decrease or a lack of increase in fibrosis and stiffness of wall and valve of the vein affected by DVT. In some embodiments, the decrease or the lack of increase in fibrosis and stiffness of wall and valve is measured by ultrasound. In some embodiments, the therapeutic delivering catheter comprises a needle injection catheter. In some embodiments, the vein affected by DVT comprises a plurality of thrombotic segments. In some embodiments, the therapeutic composition is delivered to the plurality of thrombosed segments. In some embodiments, the vein affected by DVT has undergone an endovascular procedure previously, wherein the endovascular procedures comprise one or more of venous valve repair, venous bypass, and venous stents. In some embodiments, the resolution of the thrombus takes at least 1 day, 3 days, 7 days, or 14 days. In some embodiments, the delivery of the therapeutic composition results in a maintenance or an increase in patency of the thrombosed segment. In some embodiments, the maintenance or the increase in patency lasts for at least 5 weeks, 3 months, 6 months, 12 months, 18 months, or 24 months. In some embodiments, a level of one or more anti-inflammatory biomarkers increases after the delivery of a therapeutic composition into a perivascular tissue at or near the thrombosed segment. In some embodiments, the one or more anti-inflammatory biomarkers comprises one or more of IL-10 and IL-1 receptor antagonist (IL-1 Ra). In some embodiments, the reduction in progression to PTS is assessed by maintenance or an increase in patency of the thrombosed segment. In some embodiments, the maintenance or the increase in patency lasts for at least 5 weeks, 3 months, 6 months, 12 months, 18 months, or 24 months. In some embodiments, the reduction in progression to PTS is assessed by a decrease or a lack of increase in rethrombosis in the thrombosed segment. In some embodiments, the decrease or the lack of increase in rethrombosis lasts for at least 5 weeks, 3 months, 6 months, 12 months, 18 months, or 24 months. In some embodiments, the decrease or the lack of increase in rethrombosis is measured by ultrasound. In some embodiments, the reduction in progression to PTS is assessed by a decrease or a lack of increase in a Villalta score or a VCSS score. In some embodiments, the vein affected by DVT currently or previously and/or is at risk for progressing to PTS is identified by fluordeoxyglucose-positron emission tomography (FDG-PET). In some embodiments, the reduction in progression to PTS is assessed by FDG-PET scanning of the perivascular tissue. In some embodiments, the FDG-PET detects a level of local metabolic activity in the perivascular tissue. In some embodiments, the level of local metabolic activity indicates localized inflammation. In some embodiments, an increase in a residual local metabolic activity detected by FDG-PET indicates progression to PTS. In some embodiments, a decrease in a residual local metabolic activity detected by FDG-PET indicates reduction in progression to PTS. In some embodiments, the therapeutic composition comprises one or more component for extended release, sustained release, or controlled release. In some embodiments, the therapeutic composition is extended released, sustained released, or controlled released in the perivascular tissue.

Provided herein are methods of reducing progression to post-thrombotic syndrome (PTS) in a subject by reducing MMP-9 level in a perivascular tissue around a vein affected by deep vein thrombosis (DVT), the method comprising: (a) identifying a vein in the subject affected by DVT currently or previously and/or is at risk for progressing to PTS; (b) advancing a therapeutic delivering catheter within a lumen of the vein affected by DVT to or near a thrombosed segment of the vein; and (c) delivering a therapeutic composition into a perivascular tissue at or near the thrombosed segment using the therapeutic delivering catheter, wherein the therapeutic composition comprises one or more of a corticosteroid, a MMP-9 inhibitor, and an agent capable of reducing a level of MMP-9 or another MMPs. In some embodiments, the vein affected by DVT comprises a plurality of thrombotic segments. In some embodiments, the therapeutic composition is delivered to the plurality of thrombosed segments. In some embodiments, the vein affected by DVT has undergone a catheter-directed thrombolysis or thrombectomy (CDT) previously. In some embodiments, the therapeutic composition comprises one or more component for extended release, sustained release, or controlled release. In some embodiments, the delivery of the therapeutic composition results in a resolution of a thrombus in the thrombosed segment. In some embodiments, the resolution of the thrombus takes at least 1 day, 3 days, 7 days, or 14 days. In some embodiments, a level of one or more inflammatory biomarkers decreases after the delivery of a therapeutic composition into a perivascular tissue at or near the thrombosed segment, wherein the one or more inflammatory biomarkers comprises one or more of IL-1β, IL-2, IL-6, IL-8, IL-10, IFN-α, IFN-γ, ICAM-1, TNF-α, CRP, D-dimer, fibrinogen, MCP-1, IL-1Ra, IL-1α, MMP-1, MMP-2, MMP-8, MMP-9, TIMP, ICAM-1, VCAM-1, and soluble P-selectin. In some embodiments, the reduction in progression to PTS is assessed by a decrease or a lack of increase in venous reflux. In some embodiments, the decrease or the lack of increase in venous reflux lasts for at least 5 weeks, 3 months, 6 months, 12 months, 18 months, or 24 months. In some embodiments, the decrease or the lack of increase in venous reflux is measured by ultrasound. In some embodiments, the reduction in progression to PTS is assessed by a decrease or a lack of increase in fibrosis and stiffness of wall and valve of the vein affected by DVT. In some embodiments, the decrease or the lack of increase in fibrosis and stiffness of wall and valve is measured by ultrasound. In some embodiments, the reduction in progression to PTS is assessed by a decrease or a lack of increase in a symptom of PTS, wherein the symptom of PTS comprises one or more of pain, cramps, heaviness, pruritus, paresthesia, edema, skin induration, hyperpigmentation, venous ectasia, redness, and pain during calf compression. In some embodiments, the therapeutic delivering catheter comprises a needle injection catheter. In some embodiments, the reduction in progression to PTS is assessed by a decrease or a lack of increase in a symptom of PTS, wherein the symptom of PTS comprises one or more of pain, cramps, heaviness, pruritus, paresthesia, edema, skin induration, hyperpigmentation, venous ectasia, redness, and pain during calf compression. In some embodiments, the reduction in progression to PTS is assessed by a decrease or a lack of increase in venous reflux. In some embodiments, the decrease or the lack of increase in venous reflux lasts for at least 5 weeks, 3 months, 6 months, 12 months, 18 months, or 24 months. In some embodiments, the decrease or the lack of increase in venous reflux is measured by ultrasound. In some embodiments, the reduction in progression to PTS is assessed by a decrease or a lack of increase in fibrosis and stiffness of wall and valve of the vein affected by DVT. In some embodiments, the decrease or the lack of increase in fibrosis and stiffness of wall and valve is measured by ultrasound. In some embodiments, the therapeutic delivering catheter comprises a needle injection catheter. In some embodiments, the vein affected by DVT comprises a plurality of thrombotic segments. In some embodiments, the therapeutic composition is delivered to the plurality of thrombosed segments. In some embodiments, the vein affected by DVT has undergone an endovascular procedure previously, wherein the endovascular procedures comprise one or more of venous valve repair, venous bypass, and venous stents. In some embodiments, the resolution of the thrombus takes at least 1 day, 3 days, 7 days, or 14 days. In some embodiments, the delivery of the therapeutic composition results in a maintenance or an increase in patency of the thrombosed segment. In some embodiments, the maintenance or the increase in patency lasts for at least 5 weeks, 3 months, 6 months, 12 months, 18 months, or 24 months. In some embodiments, a level of one or more anti-inflammatory biomarkers increases after the delivery of a therapeutic composition into a perivascular tissue at or near the thrombosed segment. In some embodiments, the one or more anti-inflammatory biomarkers comprises one or more of IL-10 and IL-1 receptor antagonist (IL-1 Ra). In some embodiments, the reduction in progression to PTS is assessed by maintenance or an increase in patency of the thrombosed segment. In some embodiments, the maintenance or the increase in patency lasts for at least 5 weeks, 3 months, 6 months, 12 months, 18 months, or 24 months. In some embodiments, the reduction in progression to PTS is assessed by a decrease or a lack of increase in rethrombosis in the thrombosed segment. In some embodiments, the decrease or the lack of increase in rethrombosis lasts for at least 5 weeks, 3 months, 6 months, 12 months, 18 months, or 24 months. In some embodiments, the decrease or the lack of increase in rethrombosis is measured by ultrasound. In some embodiments, the reduction in progression to PTS is assessed by a decrease or a lack of increase in a Villalta score or a VCSS score. In some embodiments, the vein affected by DVT currently or previously and/or is at risk for progressing to PTS is identified by fluordeoxyglucose-positron emission tomography (FDG-PET). In some embodiments, the reduction in progression to PTS is assessed by FDG-PET scanning of the perivascular tissue. In some embodiments, the FDG-PET detects a level of local metabolic activity in the perivascular tissue. In some embodiments, the level of local metabolic activity indicates localized inflammation. In some embodiments, an increase in a residual local metabolic activity detected by FDG-PET indicates progression to PTS. In some embodiments, a decrease in a residual local metabolic activity detected by FDG-PET indicates reduction in progression to PTS. In some embodiments, the therapeutic composition comprises one or more component for extended release, sustained release, or controlled release. In some embodiments, the therapeutic composition is extended released, sustained released, or controlled released in the perivascular tissue. In some embodiments, the therapeutic delivering catheter accesses the vein affected by DVT from a popliteal vein.

Provided herein are systems for use in reducing progression to post-thrombotic syndrome (PTS) in a subject, the system comprising: a therapeutic composition comprising an anti-inflammatory agent; a catheter configured to be placed within a vein affected by deep vein thrombosis (DVT) in the subject; an expandable element at a distal end of the catheter, wherein the expandable element is inflatable from an involuted contracted configuration; and an injection needle coupled to the expandable element, wherein expanding the expandable element advances the injection needle in a direction transverse to a longitudinal axis of the catheter to puncture wall of the vein at or near a thrombosed segment of the vein, and wherein, when the needle has punctured the wall of the vein, the needle delivers an amount of the therapeutic composition to a perivascular tissue at or near a thrombosed segment of the vein, the amount being therapeutic to reducing progression to PTS. In some embodiments, the therapeutic composition comprises a fibrinolytic agent. In some embodiments, the therapeutic composition comprises one or more component for extended release, sustained release, or controlled release. In some embodiments, the vein affected by DVT has undergone a catheter-directed thrombolysis or thrombectomy (CDT) previously. In some embodiments, the vein affected by DVT comprises a plurality of thrombotic segments. In some embodiments, the expandable element is expandable to a circumference to fill a lumen of the vein, wherein the circumference is larger than 2 mm.

Described herein are compositions comprising an anti-inflammatory agent for use in a method of reducing progression to post-thrombotic syndrome (PTS), wherein: said method comprises delivery of said composition into a perivascular tissue at or near a thrombosed section of a vein affected by deep vein thrombosis (DVT) currently or previously and/or is at risk for progressing to PTS; and the composition comprises a dose of the anti-inflammatory agent from about 0.1 mg per cm of the thrombosed segment to about 10 mg per cm of the thrombosed segment. In some embodiments, the anti-inflammatory agent: comprises a glucocorticoid, preferably dexamethasone; and/or further comprises a fibrinolytic agent.

Provided herein are compositions comprising mononuclear cells or stem-like cells for use in a method of reducing progression to post-thrombotic syndrome (PTS), wherein said method comprises delivery of said composition into a perivascular tissue at or near a thrombosed section of a vein affected by deep vein thrombosis (DVT) currently or previously and/or is at risk for progressing to PTS.

Provided herein are compositions for use in a method of reducing progression to post-thrombotic syndrome (PTS), wherein: said method comprises delivery of said composition into a perivascular tissue at or near a thrombosed section of a vein affected by deep vein thrombosis (DVT) currently or previously and/or is at risk for progressing to PTS; and the composition comprises one or more of a corticosteroid, a MMP-9 inhibitor, and an agent capable of reducing a level of MMP-9 or another MMPs.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Some understanding of the features and advantages of the present disclosure will be obtained by reference to the following detailed description that sets forth illustrative embodiments, in which the principles of the invention are utilized, and the accompanying drawings of which:

FIG. 1 shows a schematic of the interaction between cytokines, chemokines, adhesion molecules, MMPs, cells, and coagulation activation in pathophysiology of thrombus formation.

FIG. 2 shows a schematic of a vein after thrombectomy and stenting in a patient experiencing post-thrombotic syndrome (PTS).

FIG. 3 shows a schematic of a hypothesis of pathways involved in progressing from DVT to PTS.

FIG. 4 shows graphs of experimental results of MMP-9 plasma concentration over time before and after dexamethasone injection.

FIG. 5 shows a schematic of placement of a radiographic ruler on thigh to allow measurement and indexing.

FIG. 6 shows a graphical example of a needle injection catheter having a balloon that sheaths a microneedle.

FIG. 7 shows a graphical example of a needle injection catheter delivering a therapeutic composition into the perivascular space of a vein affected by DVT.

FIG. 8 is a schematic, perspective view of a medical instrument for localized drug delivery in accordance with some embodiments of the disclosure.

FIG. 9 is an enlarged view showing portion A of FIG. 8.

FIG. 10A shows the medical instrument for localized drug delivery where a tissue penetrating member is not yet deployed in accordance with some embodiments of the disclosure.

FIG. 10B is a cross-sectional view along line A-A of FIG. 10A.

FIG. 11A shows an exemplary medical instrument for localized drug delivery where an inflatable body is at a partially inflated configuration in accordance with some embodiments of the disclosure.

FIG. 11B is a cross-sectional view along line B-B of FIG. 11A, showing a transitional configuration toward the partially inflated configuration of inflatable body.

FIG. 11C is a cross-sectional view along line B-B of FIG. 11A, showing the partially inflated configuration of inflatable body.

FIG. 12A shows the medical instrument for localized drug delivery where the inflatable body is at a fully inflated configuration and the tissue penetrating member is deployed in accordance with some embodiments of the disclosure.

FIG. 12B is a cross-sectional view along line C-C of FIG. 12A.

FIG. 13A is a schematic, perspective view of the medical instrument for localized drug delivery as being inserted into a patient's body lumen in accordance with some embodiments of the disclosure.

FIG. 13B is a schematic, perspective view of the medical instrument for localized drug delivery as the tissue penetrating member is deployed in the patient's body lumen in accordance with some embodiments of the disclosure.

FIG. 13C is a schematic, perspective view of the medical instrument for localized drug delivery as the tissue penetrating member penetrating into a luminal wall of the patient's body lumen in accordance with some embodiments of the disclosure.

FIG. 14A is a cross-sectional view of the junction between three-lumen catheter tubing and the three fluid paths created by use of elastomeric coating and vapor polymer deposition, in accordance with some embodiments of the disclosure.

FIG. 14B is a cross-sectional view along line D-D of FIG. 14A.

FIG. 14C is a cross-sectional view of a dissolvable mold element used to create the junction in FIG. 14A.

FIG. 14D is an assembly consisting of the dissolvable mold element and tubing used to create the junction in FIG. 14A.

FIG. 15 shows a flow chart of a method for delivering a drug to a patient in accordance with some embodiments of the disclosure.

FIG. 16 shows a graph of RNA analysis result of inflammation panel in an in vivo murine study.

FIG. 17 shows a graph of RNA analysis result of fibrosis-related gene panel in an in vivo murine study.

FIG. 18 shows representative histology images of the IVC and DVT in an in vivo murine study.

FIG. 19 shows a graph showing percentage of the thrombus area occupied by organizing thrombus in an in vivo mouse study. The area of organizing thrombus in the dexamethasone-treated group was significantly smaller than in the control group (p=0.024).

FIG. 20 shows graphs depicting a semi-quantitative evaluation of inflammation in the entire thrombus. in an in vivo mouse study. More severe inflammation was observed in the control group compared to the dexamethasone-treated groups. There were no significant differences in terms of the distribution of inflammation in the thrombus.

FIG. 21 shows graphs depicting dexamethasone levels measured in pig carotid arteries 1, 4, and 7 days after confirmed delivery of 1 mg dexamethasone sodium phosphate in 3 ml volume to the carotid artery adventitia with the Bullfrog Micro-infusion Device from an in vivo pig study. The delivery was made in segment 3 in each case. each line represents a single artery.

FIG. 22 shows an FDG-PET scan of leg with DVT in comparison to a leg without DVT.

FIG. 23 shows data (SUVmax) indicating the local metabolic activity due to localized inflammation in veins with DVT in comparison to contralateral, non-DVT veins or in comparison to normal limbs in patients without DVT.

### DETAILED DESCRIPTION

Disclosed herein are device, methods, and kits for reducing symptoms of and treatment of post-thrombotic syndrome (PTS) in an individual. Often, PTS may result from deep vein thrombosis (DVT) or blood clots in a vein in an individual. Provided herein are device, methods, and kits to reduce or resolve inflammation that is present during venous thrombosis, including but not limited to DVT and or pulmonary embolism (PE), or after treatment of venous thrombosis.

Often, individuals having PTS have an elevated or altered level of inflammation. In some cases, inflammation may arise prior to clot formation and may be exacerbated by the organization of the thrombus. In some cases, inflammation may be increased after mechanical, surgical, and/or endovascular procedures to remove the clot. In some cases, the local inflammation may have been caused by thrombosis and thrombectomy. In some cases, the inflammation may be an acute inflammation that is elevated for a short time. In some cases, the inflammation may be a subacute inflammation or a chronic inflammation that persists for more than 2 weeks. Locally delivered treatment to reduce the various causes of inflammation found in individuals with PTS may help treat PTS and reduce PTS symptoms.

In some cases, steroids, corticosteroids, glucocorticoids, or other agents with anti-inflammatory properties may be used to decrease the local inflammation at or near the site of PTS. Sometimes, delivery of glucocorticoids, dexamethasone, dexamethasone sodium phosphate, or equipotent doses of other glucocorticoids may aid in the resolution of inflammation. Usually, the delivery of these agents directly into perivascular tissues around the vein or artery that has been thrombosed can reduce the local inflammation by reducing the level of several factors associated with inflammation, including but not limited to MCP-1, IL-6, IL-1α, MMP-1, MMP-2, MMP-8, MMP-9, TIMP, TNF-α, ICAM-1, VCAM-1, and soluble P-selectin. In some cases, dexamethasone and other glucocorticoids may increase the expression of anti-inflammatory cytokines, including but not limited to IL-10 and IL-1 receptor antagonist (IL-1 Ra).

Usually, the systemic levels of factors and cytokines may be measurable from a blood sample. In some cases, the blood sample may be a whole blood sample, serum, or plasma. Often, the injection of dexamethasone, glucocorticoids, corticosteroids, or other agents with anti-inflammatory properties may result in a measurable change in the levels of the factors and cytokines. In some cases, the measurable change is the systemic levels of the factors and cytokines from a blood sample.

In some cases, when a glucocorticoid is delivered together with tissue plasminogen activator (tPA) by direct injection into organized thrombus, the reaction of the glucocorticoid to increase plasminogen activator inhibor-1 can be counterbalanced. In some cases, the counterbalancing may be achieved by delivering the glucocorticoid to the outside of the vessel wall and delivering the tPA inside the vessel into the organizing thrombus. In some cases, the counterbalancing may be achieved by delivering the glucocorticoid to a first site in the vessel wall and delivering the tPA to a second site near the first site.

In some cases, various cytokines, adhesion molecules, and matrix metalloproteinases may be used as a predisposing, diagnostic, or prognostic factors for venous thrombosis, DVT, PE, or PTS. Tables 1-3 provide non-limiting lists of these molecules and their activities. Table 1 shows a non-limiting list of cytokines as predisposing factors, diagnostic markers, and prognostic markers for venous thrombosis. Table 2 shows a non-limiting list of adhesion molecules as predisposing factors, diagnostic markers, and prognostic markers for venous thrombosis. Table 3 shows a non-limiting list of matrix metalloproteases as predisposing factors, diagnostic markers, and prognostic markers for venous thrombosis. Additional factors are described in publication by Mosevoll et al, 2018 (Mosevoll K A, Johansen S, Wendelbo Ø, Nepstad I, Bruserud Ø, Reikvam H. Cytokines, Adhesion Molecules, and Matrix Metalloproteases as Predisposing, Diagnostic, and Prognostic Factors in Venous Thrombosis. Front Med (Lausanne). 2018 May 22;5:147.), which is incorporated by reference.

In some cases, fluorodeoxyglucose-positron emission tomography (FDG-PET) may be used to detect high levels of metabolic activity in the body, which indicates localized inflammation and can be used as a predisposing, diagnostic, or prognostic factor for venous thrombosis, DVT, PE, or PTS (as further described in Rondina M T, Lam U T, Pendleton R C, Kraiss L W, Wanner N, Zimmerman G A, Hoffman J M, Hanrahan C, Boucher K, Christian P E, Butterfield R I, Morton K A. (18)F-FDG PET in the evaluation of acuity of deep vein thrombosis. Clin Nucl Med. 2012 December; 37(12):1139-45. doi: 10.1097/RLU.0b013e3182638934. PMID: 23154470; PMCID: PMC3564643.). In some cases, the perivascular edema or tissue constituent fluids may be assessed using Mill, CT, FDG-PET, ultrasound, or other non-invasive imaging modalities.

Often, methods to reduce local inflammation of the venous segment affected by DVT may likely to reduce venous re-occlusion and progression to PTS after removal of thrombus. In some embodiments, local perivascular delivery of an anti-inflammatory agent, such as dexamethasone, may improve long-term clinical outcomes in iliofemoral and femoropopliteal DVT. In some embodiments, the purposes of localized drug therapy to reduce progression to PTS and to relieve symptoms of PTS provided herein are (1) to treat or resolve the clot, which may be acute or organized, and (2) to resolve and prevent further inflammatory signaling that may lead to fibrosis of the vein wall and subsequent PTS. In some embodiments, methods to reduce local inflammation of the venous segment may be likely to reduce progression to PTS after removal of thrombus. In some embodiments, methods to reduce local inflammation of the venous segment may reduce stent thrombosis in venous stents. In some embodiments, the local fibrinolytic therapy delivered directly into the resistant (organized) thrombus may aid with the resolution of the clot. In some embodiments, local, perivascular delivery of an anti-inflammatory agent such as dexamethasone may improve long-term clinical outcomes in iliofemoral and femoral-popliteal DVT. In some embodiments, such local, perivascular delivery of an anti-inflammatory agent such as dexamethasone may be paired with intra-thrombus injection of tissue plasminogen activator (tPA) to assist with clot resolution.

The methods, therapeutic uses, devices, systems, and kits described herein have many advantages in treating the inflammation present in patients with PTS. The methods, therapeutic uses, devices, systems, and kits described herein provide local delivery of a therapeutic composition comprising one or more of steroids, corticosteroids, glucocorticoids, and other agents with anti-inflammatory properties to the affected site experiencing inflammation from PTS. In some cases, the methods, therapeutic uses, devices, systems, and kits provided use a large balloon to allow for a more accurate access and delivery in veins, which have a larger lumen than an artery. In some cases, the therapeutic composition comprises a fibrinolytic agent. In some cases, the therapeutic composition comprises mononuclear stem or stem-like cells. In some cases, the goal of local delivery of the therapeutic composition into the thrombotic segments may be to reduce inflammation and extend vein patency. In some cases, an entire segment of vein can be treated by moving the device around and targeting the needle for delivery in different segments where thick, adherent clot is not present. In some cases, where thick, adherent clot or organized thrombus is present, a fibrinolytic, anti-platelet, or anti-coagulant agent, such as tPA, may be delivered directly into the organized tissue in combination with an anti-inflammatory agent, which aids with the resolution of the thrombus. While various fibrinolytic agents and anti-inflammatory agents are commercially available, the local delivery of therapeutic compositions comprising these agents have not been used in treating affected veins in subjects at risks for PTS. Systemic corticosteroid therapy has not been used as a treatment for DVT potentially because long-term systemic corticosteroid therapy has been linked to thromboembolic events; however, the localized administration of short term bursts of corticosteroid therapy have not been similarly linked to clotting events. Thus, local administration of corticosteroid therapy for a short duration may provide significant advantages over systemic administration or longer term treatment duration to reduce rates of progression to PTS and symptoms associated with PTS. In some embodiments, such local administration of therapeutics for short duration may reduce systemic side effect, allow for delivery of a lower amount than for systemic administration while achieving therapeutic efficacy, and/or longer residence time of the therapeutic in the tissue at or near the delivery site.. In some embodiments, the local administration of therapeutics for short duration may reduce systemic side effect due to a lower amount that needs to be delivered for direct, local administration than for systemic administration to achieve therapeutic efficacy. In some embodiments, the lower amount by direct, local administration allows for reduced systemic toxicity and side effects. In some embodiments, the direct, local injection of therapeutics into the perivascular tissue allows for a longer residence time of the therapeutic in the tissue at or near the injection site than for systemic delivery. In some embodiments, the longer residence time of the therapeutic in the tissue by direct, local injection is at least 3 days, 7 days, 14, days, 21 days, 28 days, 1 month, 2 months, 3 months, 4 months, 5 months, or 6 months as compared to systemic administration of the same amount of therapeutic.

TABLE 1
Cytokines as predisposing factors, diagnostic markers,
and prognostic markers in venous thrombosis.

| | | Acute reaction and diagnostic | Effect on thrombus |
|---|---|---|---|
| Predisposing factor | | use | resolution |
| IL-1α | −899C/T ↓ SNP: 108 | | |
| | DVT vs. 325 controls | | |
| IL-1β | Rs1143634 ↓ SNP in | | |
| | DVT in larger cohort | | |
| | (4) ↔506 DVT vs. | | |
| | 1464 controls | | |
| | IL1RN-H5H5 ↑ Leiden | | |
| | thrombophilia study | | |
| IL-4 | −589 T allele ↑ SNP: | | |
| | 108 DVT vs. 325 | | |
| | controls | | |
| IL-6 | ↔ 506 DVT bs. 1464 | −174 G > C ↔ 128 DVT, 105 PE | ↑ 182 recurrent VTE vs. |
| | controls | vs. 122 controls | 350 controls |
| | −174 CC ↑ SNP: 108 | ↑ 84 VTE vs. 100 controls | ↑ in post-thrombotic |
| | VTE vs. 325 controls | ↑ 49 VTE vs. 48 controls | syndrome, 49 DVT |
| | (36) | ↑ 40 DVT- vs. 33 DVT⁻ | ↑ in post-thrombotic |
| | −174 G > C ↑ SNP: 130 | ↑ 201 DVT vs. 60 controls | syndrome, 136 DVT |
| | DVT⁺ and 190 DVT⁻ | ↑ abdominal cancer, post-operative | (mice) |
| | (cancer patients) vs. | [40 DVT vs. 40 non-DVT vs. 40 | ↑ in post-thrombotic |
| | 215 controls | controls] | syndrome, 387 DVT |
| | −174 GC ↑ SNP: 119 | ↔ 181 cases vs. 313 controls | ↑ risk for post- |
| | VTE vs. 126 controls | ↑ 68 cases vs. 67 controls | thrombotic syndrome, |
| | −174 G > C ↔ SNP: | | 110 DVT patients |
| | 128 DVT, 105 PE vs. | | ↑ 201 DVT vs. 60 |
| | 122 controls ↔ IL6: | | controls |
| | 128 DVT, 105 PE vs. | | ↔ 181 cases vs. 313 |
| | 122 controls | | controls |
| | CC −572 G/C ↑ 140/246 | | ↑43 DVT vs. 43 controls |
| | VTE vs. 160/292 | | ↑ increased risk for post- |
| | controls, respectively | | thrombotic syndrome. |
| | ↑IL6, 200 ovarian | | 803 participants SOX |
| | cancer, predictor for | | trial |
| | VTE | | |
| | ↑IL6 in 34 VTE 322 | | |
| | patients with diffuse | | |
| | large B-cell lymphoma | | |
| CXCL8/ | ↔ 506 VTE vs. 1464 | ↑ 49 VTE vs. 48 controls | ↑ 182 recurrent VTE vs. |
| IL-8 | controls | ↑ 40 DVT⁺ vs. 33 DVT⁻ | 350 controls |
| | −251AT ↑ SNP: 119 | ↔ 181 cases vs. 313 controls | ↔ 181 cases vs. 313 |
| | VTE vs. 126 controls | | controls |
| | ↑ 474 DVT vs. 474 | | ↑43 DVT vs. 43 controls |
| | controls | | Correlation between |
| | | | baseline lumen diameter |
| | | | of the femoral thrombi |
| | | | and IL-8 cytokine |
| | | | ↔ risk for post- |
| | | | thrombotic syndrome, |
| | | | 387 DVT |
| IL-10 | ↓ in VTE group in | ↓ abdominal cancer, post-operative | ↔ 181 cases vs. 313 |
| | trauma cohort | 40 DVT vs. 40 non-DVT vs. 40 | controls |
| | ↔ 506 VTE vs. 1464 | controls | ↓ 43 DVT vs. 43 |
| | controls | ↔ 181 cases vs. 313 controls (50) | controls |
| | Rs1800872 ↑ SNP IL- | | ↑ increased risk for post- |
| | 10 in DVT cohort (22 | | thrombotic syndrome, |
| | 413 women) | | 803 participants SOX |
| | −1082GG genotype ↓ in | | trial |
| | 660 DVT vs. 660 | | ↔ risk for post- |
| | controls | | thrombotic syndrome, |
| | ↑IL10 in 34 VTE 322 | | 387 DVT |
| | patients with diffuse | | |
| | large B-cell lymphoma | | |
| IL-12p70 | ↔ 506 VTE vs. 1464 | | |
| | controls | | |
| IL-13 | ↑ TT genotype: 108 | | |
| | VTE vs. 325 controls | | |
| | (female) | | |
| CCL2/ | −2518AG ↑ SNP: 119 | ↔ 49 VTE vs. 48 controls | ↑43 DVT vs. 43 controls |
| MCP-1 | VTE vs. 126 controls | ↑ 201 DVT vs. 60 controls | |
| | | ↑ 68 patients vs. 67 controls | |
| TNF-α | ↑ TNF-α in VTE in | ↔ 49 VTE vs. 48 controls | ↑43 DVT vs. 43 controls |
| | cancer cohort | ↑ 201 DVT vs. 60 controls | |
| | ↑ TNF-α and TNFA | ↑ 68 patients vs. 67 controls | |
| | haplotype in 15 VTE in | | |
| | cancer cohort 157 GI | | |
| | cancer and controls 157 | | |
| | ↑ −308A allele 68 | | |
| | patients vs. 62 controls | | |
| IFN-γ | | | ↑ IFN-γ enhances |
| | | | thrombus resolution in |
| | | | mice through enhanced |
| | | | MMP9 and VEGF |
| | | | expression in mice |
| TNFSF4 | SNP ↑ (921C > T), ↓ | | |
| | (rs3850641) 344 DVT | | |
| | vs. 2269 controls | | |
| NF-kB | | ↑abdominal cancer, post-operative | |
| | | 40 DVT vs. 40 non-DVT controls | |
| TGF-β1 | | ↔181 cases vs. 313 controls | ↔181 cases vs. 313 |
| TGF-β2 | | | controls |
| | | | MATS 42 recurrent |
| | | | DVT vs. 84 controls |
| PDGF | | ↔181 cases vs. 313 controls | ↔181 cases vs. 313 |
| | | | controls |
| Multiplex | | IL1RA, EGF, HGF CXCL5, | |
| analysis | | CXCL10, and Leptin ↑21 DVT vs. | |
| | | 20 controls | |
| | | IL1-α, IL-β, IL-2, IL-4, IL-5, IL-6, | |
| | | IL-7, IL-22, IL1RA, CCL-3/4/5/11, | |
| | | CXCL-5/10/11, bFGF, G-CSF, | |
| | | GM-CSF, VEGF, TPO, EGF, HGF, | |
| | | and Leptin, IFN-γ CD40L, TNF- α | |
| | | ↔21 DVT vs. 20 controls | |

This table summarizes selected key human and animal studies of c response in venous thrombosis. Arrows indicate the following: the cytokine/genetic polymorphism coding for the cytokine is elevated/more frequent (↑), decreased/less frequent (↓), or unchanged (↔) in DVT cohorts as a predisposing factor (left column), as part of the acute reaction (middle column), or as a risk factor for post-thrombotic syndrome or recurrent DVT (right column). PTS, post thrombotic syndrome; SNP, single nucleotide polymorphism; TIMP, tissue inhibitor of metalloproteases. Control, healthy control.

TABLE 2
Adhesion molecules as predisposing factors, diagnostic
markers and prognostic markers in venous thrombosis.

| | | Acute reaction and diagnostic | Effect on thrombus |
|---|---|---|---|
| Predisposing factor | | use | resolution |
| P-selectin | ↔ in DVT | ↑, meta-analysis 586 DVT, 1,843 | ↑ in acute DVT predicts |
| | group in | controls | post-thrombotic syndrome, |
| | trauma cohort | ↑, lower extremity: 112 DVT vs. 122 | 49 DVT |
| | ↔ selectin | non-DVT | ↑ After anticoagulation |
| | haplotypes in | ↔, upper extremity: 32 DVT vs. 13 | therapy, possible |
| | Leiden | non-DVT | therapeutic target? |
| | Thrombophilia | ↑ 62 DVT vs. 116 non-DVT | ↓ 1 month after DVT: |
| | Study | ↑ in DVT patients vs. controls | patients with chronic |
| | | ↑ 49 VTE vs. 48 controls | thrombosis vs. in patients |
| | | ↑ 22 DVT vs. 21 non-DVT vs. 30 | with resolved |
| | | controls | P-selectin inhibition |
| | | ↔ 37 DVT vs. 32 non-DVT | decreases post-thrombotic |
| | | ↑ 52 DVT vs. 83 non-DVT | vein wall fibrosis in a rat |
| | | ↑ platelet expressing P-selectin in post- | model |
| | | operative DVT | P-selectin inhibition |
| | | ↑ 89 DVT vs. 126 controls | enhances thrombus |
| | | ↑21 DVT vs. 68 non-DVT | resolution and decreases |
| | | | vein wall fibrosis in a rat |
| | | | model |
| | | | P-selectin/PSGL inhibitors |
| | | | equal enoxaparin in VTE |
| | | | treatment |
| ICAM-1 | | ↔ ICAM-1 37 DVT vs. 32 non-DVT | ↑ risk for post-thrombotic |
| | | ↑ 181 cases vs. 313 controls | syndrome, 387 DVT |
| | | ↔21 DVT vs. 20 controls | ↑ increased risk for post- |
| | | | thrombotic syndrome, 803 |
| | | | participants SOX trial |
| VCAM-1 | | ↔ 49 VTE vs. 48 healthy controls | ↔ risk for post-thrombotic |
| | | ↔ 37 DVT vs. 32 non-DVT | syndrome, 387 DVT |
| | | ↑ 52 DVT vs. 83 non-DVT | ↑ 181 cases vs. 313 |
| | | ↑ 181 cases vs. 313 controls | controls |
| | | ↑21 DVT vs. 68 non-DVT 20 controls | |
| E-selectin | ↔ selectin | ↔ 37 VTE vs. 32 non-VTE | |
| | haplotypes in | ↑ abdominal cancer, post-operative [40 | |
| | Leiden | DVT vs. 40 non-DVT vs. 40 controls] | |
| | Thrombophilia | ↔ 28 VTE vs. 92 non-VTE | |
| | Study | | |

This table summarizes selected key human and animal studies of adhesion molecule in venous thrombosis. Arrows indicate the following: the adhesion molecule/genetic polymorphism coding for the cytokine is elevated/more frequent (↑), decreased/less frequent (↓), or unchanged (↔) in DVT cohorts as a predisposing factor (left column), as part of the acute reaction (middle column), or as a risk factor for post-thrombotic syndrome or recurrent DVT (right column). PTS, post thrombotic syndrome; SNP, single nucleotide polymorphism; TIMP, tissue inhibitor of metalloproteases. Control, healthy control.

TABLE 3
Matrix metalloproteases as predisposing factors, diagnostic
markers and prognostic markers in venous thrombosis.

| | | Acute reaction and diagnostic | Effect on thrombus |
|---|---|---|---|
| Predisposing factor | | use | resolution |
| MMP-9 | 1,562 C > T ↑ SNP: | ↑ in VTE | ↑ IFN-γ enhances |
| | 130 DVT⁺ and 190 | | thrombus resolution in |
| | DVT⁻ (cancer | | mice through enhanced |
| | patients) vs. 215 | | MMP-9 and VEGF |
| | controls | | expression in mice |
| | | | Review: the role of |
| | | | MMPs in DVT [mouse |
| | | | models] |
| MMP-1, 2, | | ↑ MMPs: 201 DVT vs. | ↑ MMP-1/8: 47 of 201 |
| 3, 7, 8, 9 | | 60 controls | DVT developing PTS |
| TIMP-1/2 | | | |
| MMP-2, | | ↑21 DVT vs. 20 controls, | |
| 3, 7, 8, 9 | | ↔21 DVT vs. 68 non-DVT | |

This table summarizes selected key human and animal studies of MMP response in venous thrombosis. Arrows indicate the following: the MMP/genetic polymorphism coding for the cytokine is elevated/more frequent (↑), decreased/less frequent (↓), or unchanged (↔) in DVT cohorts as a predisposing factor (left column), as part of the acute reaction (middle column), or as a risk factor for post-thrombotic syndrome or recurrent DVT (right column), PTS, post thrombotic syndrome; SNP, single nucleotide polymorphism; TIMP, tissue inhibitor of metalloproteases. Control, healthy control

### Post-Thrombotic Syndrome (PTS)

Post-thrombotic syndrome (PTS) is a chronic condition that may occur in subjects who have had a deep vein thrombosis (DVT) of the leg. Often, PTS may develop in the weeks or months following a DVT. A DVT is a blockage or clot that obstructs the vein and can lead to the valves and the walls of the vein becoming damaged. Typically, the veins have small vein valves inside the lumen that ensure the blood flows correctly back toward the heart. In some patients with DVT, these fragile vein valves may become damaged easily, which may result in reflux or the blood flowing in the wrong direction. In some cases, the reflux may lead to pressure build up in the veins, especially in lower part of the legs, and result in swelling and pain. The walls of the vein may become damaged and induce vein wall fibrosis in patients with DVT. Such scarred vein walls may lack the capacity to expand as normal vein walls due to the scarring. This may result in swelling (edema) and pain in the legs when blood flow to the legs increases due to physical activities. In severe cases, the vein may be so damaged as to block off any significant blood flow to the leg.

Usually, PTS may evolve from an interplay of multiple factors: fibrotic vein wall stiffening leading to venous hypertension, continued obstruction of venous outflow due to clot and thickened vein wall, and dysfunctional or damaged venous valves leading to reflux. In some embodiments, these outcomes may be linked to venous inflammation. In some embodiments, inflammation may be key in the advancement of post-DVT patients to PTS and drugs with anti-inflammatory properties could have ability to prevent PTS. In some embodiments, the high levels of inflammatory cytokines circulating in patients progressing to PTS after DVT treatment that can both result from and lead to further vein wall injury indicate this may be drug target. In some embodiments, PTS may be characterized by inflammatory venous fibrosis localized within the thrombosed segment of vein and likely proportionate to the severity of the underlying DVT. In some embodiments, the localized venous inflammation may be detectable by fluordeoxyglucose-positron emission tomography (FDG-PET) scanning of the local tissue in comparison to the undiseased tissue in the opposite limb or in other unaffected venous tissue in the body. Furthermore, the reduction of localized venous inflammation may similarly be detected with the use of FDG-PET. In some embodiments, the perivascular edema or tissue constituent fluids may be assessed using MRI, CT, FDG-PET, ultrasound, or other non-invasive imaging modalities. In some embodiments, the perivascular edema may indicate presence of local inflammation in the tissue around the vasculature.

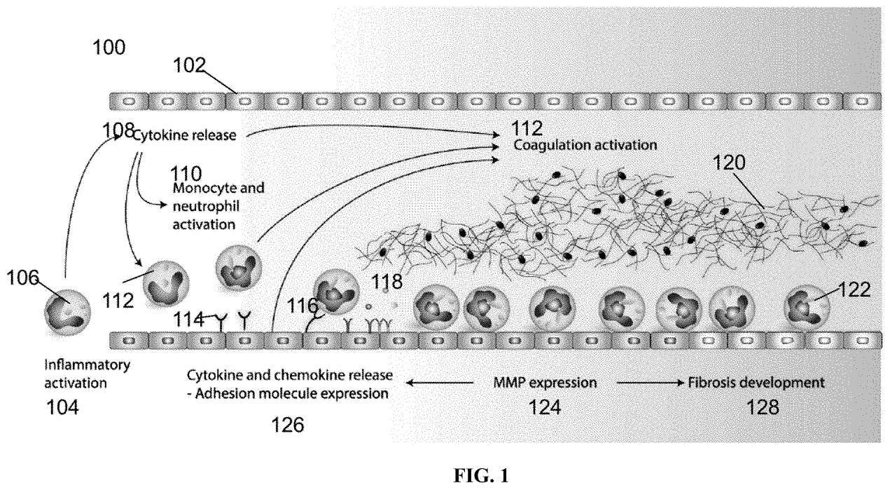

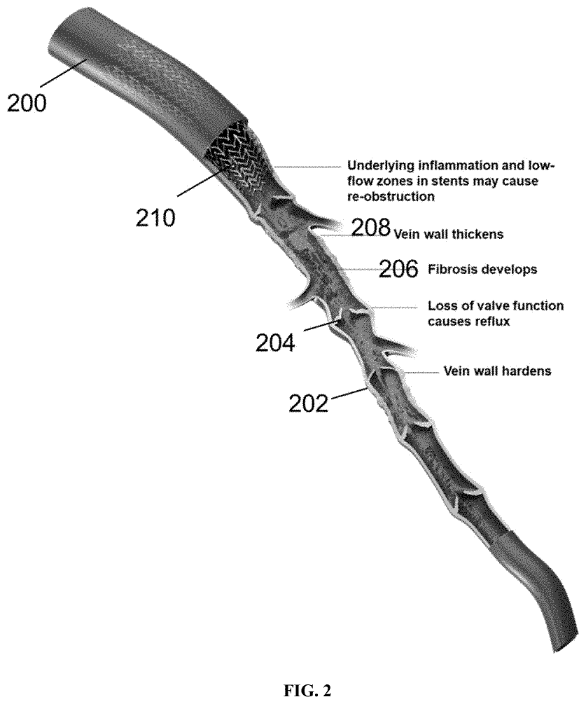

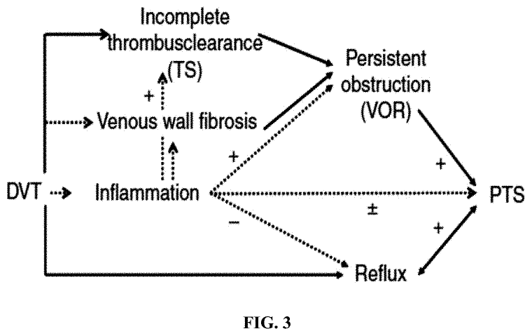

FIG. 1, from Mosevoll et al, 2018, shows a schematic of the interaction between cytokines, chemokines, adhesion molecules, MMPs, cells, and coagulation activation in pathophysiology of thrombus formation in a lumen of a vein 100 having endothelial cells along the vessel wall 102. Often, cytokines 108 may be early initiators of inflammation 104, and activated leukocytes 110, 106, 112 and endothelial cells 102 may express adhesion molecules 114 which promotes leukocyte attachment 116, 122 the endothelium 102. The cytokine release 108 may lead to coagulation activation 112, 120. The MMPs 124 may be involved in fibrosis of the vein walls 128 modulation and may act in modulation of cytokines and adhesion molecules 118, 126 during inflammation. FIG. 2 shows a schematic of a vein 200 having post-thrombotic syndrome (PTS), with underlying inflammation and low flow zones in stents 210 that may cause re-obstruction, vein wall thickening 208, development of fibrosis 206, vein wall hardening 202, and loss of vein valve functions and reflux 204. FIG. 3, from Roumen-Klappe et al, 2009 (Roumen-Klappe E M, Janssen M C, Van Rossum J, Holewijn S, Van Bokhoven M M, Kaasjager K, Wollersheim H, Den Heijer M. Inflammation in deep vein thrombosis and the development of post-thrombotic syndrome: a prospective study. J Thromb Haemost. 2009 April; 7(4):582-7. doi: 10.1111/j.1538-7836.2009.03286.x. Epub 2009 Jan. 19. PMID: 19175493.), shows a schematic of a hypothesis for pathways involved in development of PTS 314, where DVT 302 initiates an inflammatory response 304 that contribute to incomplete thrombus clearance 308, as well as vein wall changes and fibrosis 306, resulting in elevated venous outflow resistance (VOR) 310. Direct mechanical damage to the valves may contribute to venous reflux 312. Persistent obstruction 310 and venous reflux 312 may lead to venous hypertension and PTS 314.

In some cases, symptoms of PTS include but are not limited to a feeling of heaviness in the leg; itching, tingling, or cramping in the leg; leg pain that is worse with standing and better after resting or raising the leg; widening of leg veins; swelling in the leg, and darkening or redness of the skin around the leg. In some cases, PTS may result in leg ulcers due to a trauma to the leg. In some cases, PTS results in mild symptoms. In some cases, symptoms of PTS may be severe.

PTS may have various causes, including various conditions that increase chances for having DVT. The chances of having DVT increases with various events, including but are not limited to a recent surgery that decreases mobility of the subject and increases inflammation in the body, which can lead to clotting; medical conditions that limit mobility of the subject, such as an injury or stroke; long periods of travel, which limit mobility of the subject; injury to a deep vein; inherited blood disorders that increase clotting; pregnancy; and cancer treatment. The risk for having PTS may increase with various factors, including but not limited to being very overweight, having a DVT that causes symptoms, getting a thrombosis above the knee (proximal, especially with iliac or common femoral vein involvement) instead of below it (distal, such as calf), having more than one DVT, having increased pressure in the veins in the legs, and not taking blood thinners after having DVT.

While there is no gold standard biomarker, imaging, or physiologic test that establishes the diagnosis of PTS, PTS is usually diagnosed by examination of the affected leg, ultrasound to assess any problems with leg vein valves, and a blood test to assess any clotting problems with the blood of the patient. Often, Villalta score rates the severity of your symptoms (pain, cramps, heaviness, pruritus, paresthesia) and signs (edema, skin induration, hyperpigmentation, venous ectasia, redness, pain during calf compression) of PTS, where a score of >15 indicates a severe PTS. In some cases, other diagnostic or classification scales are used to assess PTS, including the CEAP classification, Ginsberg measure, and Venous Clinical Severity Score (VCSS).

PTS may be treated by one or more of lifestyle, pharmaceutical, and/or invasive or minimally invasive interventions. In some cases, symptoms of PTS may be alleviated by exercise and walking to increase leg muscle strength, elevating the affected leg, using a compression stocking or a compression device on the affected leg. In some cases, symptoms of PTS may be alleviated by taking a blood-thinning medication, such as warfarin or heparin, or a venoactive medication that affects the vessel filtration, permeability, or levels of cytokines involved in clotting. In some cases, PTS may be treated by one or more of catheter-directed thrombolysis (CDT), pharmacomechanical CDT, or an endovascular procedure such as mechanical thrombectomy, venous valve repair, venous bypass, and venous stents.

PTS is a chronic complication arising in about 30-50% of patients after treatment of proximal DVT. In some cases, PTS may be more frequently observed if DVT extends into the iliofemoral segment of the veins. In some cases, PTS may be observed within 2 years of treatment for DVT at rates of 30-40% after femoropopliteal DVT and 50-70% after iliofemoral DVT. In some cases, with or without catheter-directed thrombectomy (CDT) or pharmacomechanical catheter-directed venous thrombolysis (PCDT), there is about 40-50% rate of PTS (based on the ATTRACT, CaVenT and CAVA trials). In some cases, a complete clearance of a thrombus during a thrombolysis procedure does not appear to improve rates of progression to PTS, although severity of PTS may be reduced due to decreased residual thrombus. In some cases, PCDT may not reduce the incidence of PTS over 24 months, compared to control anticoagulation alone. In some cases, PCDT may confer reduced moderate-to-severe PTS in iliofemoral DVT, and no benefit when PCDT was administered after 8 days post-symptom onset. Overall, there remains a clear unmet need to in reducing symptoms of PTS to therapies beyond selective PCDT. The methods and the therapeutic uses provided herein may be used where the vein affected by DVT has previously undergone a CDT, and/or an endovascular procedure (examples of which are described herein).

While there is limited published data related to patency after treatment of subacute DVT, data for acute and chronic cases may be used as a point of reference. In some cases, regarding acute DVT, iliofemoral patency rates were 65.9% at 6 months among 58 patients treated with CDT, and 47.4% among 45 patients receiving standard anticoagulant therapy alone. In these subjects, 50-59% had femoral DVT, indicating involvement of the femoropopliteal segment. In some cases, chronic DVT patients may be required to take oral anticoagulants for at least 3 months pre-procedurally. After treatment with EKOS catheter thrombolysis, the total number of occluded segments at 6 months vs. baseline was reduced by a relative 100% in the CIV, 89% in EIV, 91% in CFV, 87% in Proximal FV, 86% in Distal FV, and 90% in popliteal vein. The data in this trial did not report overall patency in the subjects, so it is not known whether subjects had patency in all segments or whether there was overlap between those who had lost patency in individual segments.

On average, the United States experiences between 200,000 and 700,000 new cases of DVT each year, with estimates varying widely due to the likelihood of under-reporting. About 30%-50% of DVT patients develop morbid PTS. It is further recognized there is an increased risk for DVT with concomitant COVID-19. In some cases, there may also be a potential for thrombotic complications such as PTS in those individuals with COVID-19.

### PTS-Related Inflammation

In some embodiments, inflammation may play a role in promoting the development of PTS. In some embodiments, PTS may develop due to delayed thrombus resolution and vein wall fibrosis, which promotes valvular reflux. In some embodiments, PTS may be more closely linked to inflammation than to reobstruction. In some embodiments, inflammatory cytokines have been detected at high levels in patients progressing to PTS after DVT treatment, and signaling pathways between the thrombus and vein wall may mediate the release of inflammatory factors that lead to further vein wall injury. In some cases, PTS may be characterized by a fibrotic injury response leading to a thickened and non-compliant vein wall due to inflammation localized within the thrombosed segment of vein and may be likely proportionate to the severity of the underlying thrombosis. In some embodiments, enhancing thrombus resolution may reduce progression of PTS and symptoms of PTS. In some embodiments, reducing or inhibiting one or more cytokines in the clotting cascade may reduce progression of PTS and symptoms of PTS. In some embodiments, reducing the expression or release of inflammatory cytokines may reduce progression of PTS and symptoms of PTS. In some embodiments, reducing the fibrosis in the vein wall may reduce progression of PTS and symptoms of PTS.

In some cases, one or more inflammatory factors, including but not limited to C-reactive protein (CRP), interleukin-6 (IL-6), interleukin-8 (IL-8) and tissue necrosis factor-alpha (TNFα), may be elevated in subjects with increased risk for venous thromboembolism (VTE), a DVT subgroup. In some cases, elevation of inflammatory factors can be a cause of thrombus formation. In some cases, elevation of inflammatory factors may be a consequence of VTE and may lead to a poor resolution of thrombus, resulting in thickening and hardening of vein walls and ultimately causing progression to PTS. In some cases, reducing the levels of one or more inflammatory factors, including but not limited to CRP, IL-6, IL-8, and TNFα, may reduce progression of PTS and symptoms of PTS.

In some cases, one or more inflammatory biomarkers may have altered levels in patients with PTS. In some cases, local levels of one or more inflammatory biomarkers at or near the thrombosis site may be elevated in patients with PTS. In some cases, systemic levels of one or more inflammatory biomarkers may be elevated in patients with PTS. In some cases, one or more biomarkers may include but are not limited to IL-1β, IL-2, IL-6, IL-8, IL-10, IFN-α, IFN-γ, ICAM-1, TNF-α, CRP, D-dimer, fibrinogen, MCP-1, IL-1Ra, IL-1α, MMP-1, MMP-2, MMP-8, MMP-9, TIMP, ICAM-1, VCAM-1, and soluble P-selectin. In some cases, reducing the level of one or more inflammatory biomarkers may reduce progression of PTS and symptoms of PTS.

In some cases, one or more biomarkers may have altered levels in patients with PTS. In some cases, one or more biomarkers may be elevated in patients with PTS. In some cases, one or more biomarkers may be decreased in patients with PTS. In some cases, one or more biomarkers may be decreased while other biomarkers are elevated in patients with PTS. In some embodiments, preclinical studies have shown that matrix metalloproteinases (MMPs), specifically MMP-9, may be key regulatory cytokines in thrombus resolution. In some cases, MMP-9 expression may be increased during thrombus resolution. In some cases, a long-term elevation of MMP-9 can increase vein wall collagen, thickening and stiffening the vein wall. In some cases, elevated MMP-9 levels at late stage may be indicative of the formation of PTS, as are elevation of MMP-1 and MMP-8. In some cases, reducing the level of one or more biomarkers may reduce progression of PTS and symptoms of PTS. In some cases, reducing level of MMP-9 may alleviate symptoms of PTS and progression to PTS. In some cases, administering inhibitors of MMP-9 may alleviate symptoms of PTS and progression to PTS.

In some cases, localized metabolic activity may be detected around the venous segment experiencing DVT in patients, and residual increased local metabolic activity as detected by FDG-PET may be correlated to the development of PTS. In some cases, reducing the level of metabolic activity detectable by FDG-PET around a vein that has experienced DVT may reduce progression of PTS and symptoms of PTS.

In some embodiments, there may be about 20-30% risk of stent thrombosis after venous stenting. While many factors may contribute to thrombosis after venous stenting, inflammation may be a highly likely contributor based on the inflammatory cytokine cascade that may be local to the stent. In some embodiments, re-occlusion may be more likely to occur due to spontaneous thrombosis, induced by plasmin and other proteolytic cascades triggered by inflammatory cells present in the early thrombus.

In some cases, while the etiology of VTE may not be well elucidated, systemic drug use may contribute to negative outcomes. In some cases, use of one or more of non-selective, non-steroidal anti-inflammatory drugs (NSAIDs) and cyclooxygenase-2-selective (COX-2) inhibitors may lead to greater risk of VTE. In some cases, use of systemic, long-term corticosteroids may lead to greater risk of VTE. At least part of this outcome may be explained by the systemic use of the medications rather than their local administration.

### PTS-Related Blood Clotting

In some embodiments, PTS may result from abnormalities in clotting in the subject. In some embodiments, PTS may result in abnormal clotting in the subject. In some embodiments, glucocorticoid (GC) use has been linked to clotting in arterial and venous circulation. In some embodiments, clot formation may result from the specific imbalance amongst procoagulant, anti-coagulant, and fibrinolytic factors. In some embodiments, GCs may affect the procoagulant, anti-coagulant, and fibrinolytic factors in ways that may cause clotting in otherwise non-inflamed patients or when delivered systemically. However, a local delivery of GCs has not been linked to thrombus. In some embodiments, long-term use of GCs may increase levels of von Willebrand factor (vWF), a procoagulant factor. In some embodiments, short-term administration of GC has not shown similar increases in vWF levels. In some embodiments, in surgery, systemic GC use has been shown to decrease tissue plasminogen activator (tPA), an anti-coagulant factor, and increase plasminogen activator inhibitor-1 (PAI-1), an inhibitor of fibrinolysis.

In some embodiments, dexamethasone may affect levels of cytokines and markers involved in inflammatory responses. In some embodiments, a high dose of dexamethasone (1 mg/kg bid×2 days) induced elevated P-selectin levels in healthy males. In some embodiments, a low dose of dexamethasone (0.04 mg/kg bid×2 days) did not induce elevated P-selectin levels. In some embodiments, vWF was elevated at 24 hours and 48 hours with a dexamethasone treatment. In some embodiments, P-selectin was only elevated at 48 hours with a dexamethasone treatment.

In some embodiments, monocytes and macrophages may migrate to and resolve the clot in the vein in the presence of fibrinolytic cytokines in a subject with PTS. In some embodiments, however, the hyperactive response of the monocytes and macrophages may lead to the local inflammation, thickening and stiffening of the vein wall and valves. In some embodiments, granulocyte colony stimulating factor (G-CSF) and recombinant human G-CSF (rhG-CSF) may play a role in clot resolution. In some embodiments, the clot resolution occurs via increased release of bone marrow mononuclear cells via increased release of bone marrow mononuclear cells. In some embodiments, monocytes and macrophages (Mo/MT) may play a role in the resolution of a clot. In some embodiments, the resolution of a clot by monocytes and macrophages may be evident from histology in animals with venous thrombus and the increased levels of MCP-1 expression during clot resolution. In some embodiments, harvested or selected mononuclear, stem or stem-like cells from circulating blood, bone marrow or adipose tissue may be used to reduce clot by locally delivering the monocytes into the area of the clot, where they are useful in clot resolution.

In some embodiments, various agents that affect the coagulation cascade may reduce inflammation and/or progression of PTS in the venous segment when delivered locally to or near the affected venous segment. In some embodiments, agents that are tailored to knockout parts of the coagulation cascade and that can be locally delivered to treat PTS include but are not limited to P-selectin or E-selection inhibitors, resolvins, protectins, MMP-9 inhibitors, plasminogen activators, vWF inhibitors, low molecular heparin. In some cases, fibrinolytic, anti-platelet, or anti-coagulant agents include but are not limited to tenecteplase, reteplase, alteplase, streptokinase and urokinase. In some cases, administration of one or more agents that reduce the activity of the coagulation cascade may reduce progression of PTS and symptoms of PTS.

### Treatments to Reduce Progression to and Symptoms of PTS

Often, in subjects without treatment for DVT, their symptoms can worsen and lead to debilitating PTS. Usually, the potential to reduce the likelihood of re-thrombosis and progression to PTS after DVT intervention may help alleviate symptoms of PTS. The methods and therapeutic uses of the invention may be used to treat a vein affected by DVT which comprises a one thrombotic segment or a plurality of thrombotic segments, for example, 2, 3, 4, 5 or more thrombotic segments. A therapeutic composition may be delivered at or near to a single thrombotic segment (for example if the vein to be treated comprises a single thrombotic segment), or at or near to a plurality of thrombotic segments. In some embodiments, where a vein to be treated comprises a plurality of thrombotic segments, the therapeutic composition may be delivered at or near to each of the plurality of thrombotic segments. In some cases, the subjects have acute DVT with acute inflammation. In some cases, the subjects with acute DVT have had acute DVT symptoms for 14 days or less in the affected limb. In some cases, the subjects have subacute DVT with subacute inflammation. In some cases, the subjects have chronic DVT with chronic inflammation. In some cases, subjects with chronic DVT have a different inflammatory biomarker profile than those with acute DVT. In some cases, the DVT and inflammation may result from an extrinsic injury to the affected vein, including but not limited to surgery, trauma, accident and injury. In some cases, the DVT and inflammation may result from an intrinsic cause, including but not limited to pregnancy or edema. In some cases, the DVT and inflammation may result from an iatrogenic cause, including but not limited to cancer treatment.

In the treatment of DVT the open-vein hypothesis proposes that early and active removal of thrombus will improve deep venous flow, reduce venous reflux, and decrease the risk of PTS. However, acute DVT trials have demonstrated that progression to PTS may not be inhibited by catheter-directed thrombolysis or thrombectomy (CDT). An alternative hypothesis based on venous inflammation has arisen. In some cases, the open vein may not be enough to prevent PTS, and the residual inflammation and fibrosis of the vein wall and valves may need therapeutic attention. Thus, treatment to reduce inflammation at and near the thrombosis may be beneficial in reducing rate of progression to PTS and symptoms of PTS. As DVT and VTE are considered to be a localized disease with localized inflammation, a localized therapy may be advantageous as compared to systemic therapy in order to reduce the potential for systemic harms that can be caused by these medications.

In some embodiments, the purposes of localized drug therapy to treat DVT may include (1) treatment or resolution of the clot, which may be acute or organized, and (2) resolution and prevention of further inflammatory signaling that may lead to fibrosis of the vein wall and subsequent PTS. In some embodiments, methods to reduce local inflammation in the affected venous segment may reduce progression to PTS after removal of thrombus. In some embodiments, methods to reduce local inflammation of the venous segment may reduce stent thrombosis in venous stents. In some embodiments, the local fibrinolytic therapy delivered directly into the resistant (organized) thrombus may aid with resolution of the clot. In some embodiments, local, perivascular delivery of an anti-inflammatory agent such as dexamethasone may improve long-term clinical outcomes in DVT. In some embodiments, local, perivascular delivery of an anti-inflammatory agent such as dexamethasone may improve long-term clinical outcomes in iliofemoral and femoropopliteal DVT. In some embodiments, such local, perivascular delivery of an anti-inflammatory agent such as dexamethasone may be paired with intra-thrombus injection of tissue plasminogen activator (tPA) to assist with clot resolution.

In some cases, reducing the level of one or more inflammatory biomarkers may reduce progression of PTS and symptoms of PTS. In some cases, reducing the local level of one or more inflammatory biomarkers at or near the thrombosis site may reduce progression of PTS and symptoms of PTS. In some cases, reducing the systemic level of one or more inflammatory biomarkers may reduce progression of PTS and symptoms of PTS. In some cases, reducing the levels of one or more biomarkers, including but not limited to IL-1(3, IL-2, IL-6, IL-8, IL-10, IFN-α, IFN-γ, ICAM-1, TNF-α, hsCRP, D-dimer, fibrinogen, MCP-1, IL-1Ra, IL-1α, MMP-1, MMP-2, MMP-8, MMP-9, TIMP, ICAM-1, VCAM-1, and soluble P-selectin, may reduce progression of PTS and symptoms of PTS. In some cases, one or more biomarkers may include but are not limited to MMP-1, MMP-2, MMP-8, and MMP-9. In some cases, one or more biomarkers may include but are not limited to IL-10 and/or IL-1Ra. In some cases, reducing the levels of one or more biomarkers, including but not limited to MMP-1, MMP-2, MMP-8, and MMP-9, may reduce progression of PTS and symptoms of PTS. In some embodiments, steroids, corticosteroids, glucocorticoids, or other agents with anti-inflammatory properties may be used to reduce the levels of one or more inflammatory biomarkers, at or near the site of PTS. Sometimes, delivery of glucocorticoids, dexamethasone, dexamethasone sodium phosphate, or equipotent doses of other glucocorticoids may aid in the reduction of levels of inflammatory biomarkers.

In some cases, administration of one or more agents that reduce the activity of the coagulation cascade may reduce progression of PTS and symptoms of PTS. In some cases, reducing the level of one or more biomarkers involved in the clotting cascade may reduce progression of PTS and symptoms of PTS. In some cases, reducing the local level of one or more biomarkers involved in the clotting cascade at or near the thrombosis site may reduce progression of PTS and symptoms of PTS. In some cases, reducing the systemic level of one or more biomarkers involved in the clotting cascade may reduce progression of PTS and symptoms of PTS. In some cases, reducing the levels of one or more biomarkers involved in the clotting cascade, including but not limited to vWF inhibitors, tissue plasminogen activator (tPA), anti-platelet or anti-coagulant agents including low molecular weight heparins, and G-CSF, may reduce progression of PTS and symptoms of PTS.

In some embodiments, the local administration comprises using a percutaneous delivery device that injects the agent into the tissue surrounding the vein. In some embodiments, the device may be able to deliver drug to perivascular interstitial tissues to bathe the vein in the delivered agent.

In some embodiments, the treatment to reduce progression of PTS and/or symptoms of PTS comprises local administration of one or more anti-inflammatory agents. In some embodiments, the one or more anti-inflammatory agents comprise a glucocorticoid. In some embodiments, the one or more anti-inflammatory agents comprise dexamethasone. In some embodiments, the one or more anti-inflammatory agents comprise at least one of dexamethasone, hydrocortisone, cortisone, prednisone, prednisolone, methylprednisolone, betamethasone, triamcinolone, fludrocortisone acetate, deoxycorticosterone acetate, aldosterone, and beclomethasone. In some embodiments, the treatment to reduce progression of PTS and/or symptoms of PTS comprises local administration of one or more agents that reduce thrombus and resolve the inflammation occurring due to the localized thrombus. In some embodiments, the localized glucocorticoid administration may reduce thrombus and resolve the inflammation occurring due to the localized thrombus. In some embodiments, the treatment to reduce progression of PTS and/or symptoms of PTS comprises local administration of one or more agents that reduce local inflammation in the affected limb.

In some embodiments, the treatment to reduce progression of PTS and/or symptoms of PTS comprises local administration of one or more anti-inflammatory agents and one or more fibrinolytic agents. In some embodiments, the treatment to reduce progression of PTS and/or symptoms of PTS comprises local administration of one or more agents to reduce local inflammation and one or more agents to reduce clot formation or improve resolution of a clot. In some embodiments, the one or more fibrinolytic, anti-platelet, or anti-coagulant agents comprise at least one of tissue plasminogen activator (tPA), vWF inhibitor, low molecular weight heparin, and G-CSF. In some embodiments, the one or more fibrinolytic agents comprise tPA. Thus, a particularly preferred combination of anti-inflammatory agent and fibrinolytic agent may be dexamethasone and tPA. In some embodiments, the one or more fibrinolytic agents may be administered at or near, or directly into an acute or organizing thrombus. The delivery of a fibrinolytic agent may result in a maintenance or an increase in patency of the thrombosed segment. In some embodiments, the local administration of the combination of the one or more anti-inflammatory agents and one or more fibrinolytic agents may be administered at or near an organized thrombus in the vein in the affected limb. In some embodiments, the local administration of the combination of the one or more anti-inflammatory agents and one or more fibrinolytic agents may be administered directly into organized thrombus to resolve the thrombus. In some embodiments, the local administration of the combination of the one or more anti-inflammatory agents and one or more fibrinolytic agents may reduce the localized inflammatory reactions and resolve the thrombus, both of which contribute to progression to PTS. In some embodiments, the local administration of the combination of the one or more anti-inflammatory agents and one or more fibrinolytic agents provides a two-pronged attack of resolving the thrombus and reducing the inflammation, which may reduce vein wall thickening (scarring), preserve venous valves, and reduce the progression from DVT to PTS.

### Anti-Inflammatory Agents

Often, glucocorticoids (GCs) are utilized as immunosuppressive and anti-inflammatory agents. In some cases, one of the effects of GCs may be to exert anti-proliferative and apoptotic (i.e., programmed cell death) actions. In some cases, GCs mediate their effects by binding to the intracellular GC receptor, which can enter the nucleus of the cell, dimerize, and bind to specific DNA sequences and GC response elements thereby activating transcription of target genes. In some cases, the anti-inflammatory and immunosuppressive effects of GC may be achieved by inhibition rather than by activation of target gene expression. In some cases, many down-regulated genes involved in the inflammatory response may not contain GC response elements in their promoter. In some cases, they may be down-regulated by different mechanisms, i.e., transcriptional factors such as NF-kB. NF-kB is regulated by I-kB and GCs such as dexamethasone are potent inhibitors of NF-kB activation via enhanced I-kB gene transcription.

In some embodiments, a glucocorticoid delivered minimally invasively by catheter into the adventitia and perivascular tissue around veins that have experienced DVT and subsequently been recanalized may decrease the inflammation that could lead to rethrombosis, venous wall and valve fibrosis and stiffening and the accompanying venous reflux and hypertension. In some embodiments, one or more of these outcomes typically accompanies chronic PTS. In some embodiments, the glucocorticoid delivery may improve venous patency and reduce the rate of progression to PTS.

Dexamethasone is a generic anti-inflammatory steroid compound that may be a synthetic analog to the naturally occurring glucocorticoids cortisone and hydrocortisone. In some cases, at equipotent anti-inflammatory doses, dexamethasone may lack the sodium-retaining property of hydrocortisone and closely related derivatives of hydrocortisone. In some cases, dexamethasone may be designated chemically as 9-fluoro-11(beta),17,21-trihydroxy-16(alpha)-methylpregna-1,4-diene-3,20-dione. The empirical formula of dexamethasone is C₂₂H₂₉FO₅.

In some cases, dexamethasone may reduce the expression of one or more inflammatory cytokines. In some cases, dexamethasone may reduce the expression of one or more inflammatory cytokines, including but not limited to MMP-9, MCP-1, TNFα, CRP, IL-1β and IL-6. In some cases, dexamethasone may increase the expression of one or more anti-inflammatory cytokines, including but not limited to IL-10, which concomitantly reduces expression of MCP-1. In some cases, elevation of one or more of the inflammatory cytokines MCP-1, CRP, MMP-9 and TNFα has been directly correlated to thrombosis and PTS, so their down-regulation with dexamethasone may reduce re-thrombosis and PTS rates. In some cases, dexamethasone may have potent effects down-regulating the expression of monocyte chemoattractant protein-1 (MCP-1). In some cases, MCP-1 reduction has been shown to decrease macrophages present in atherosclerotic lesions and inhibit macrophage accumulation following balloon angioplasty in cholesterol fed rabbits. In some cases, the anti-macrophage effect of dexamethasone may support its use in vascular disease in view of the large numbers of macrophages present in human atherosclerotic lesions and in arteriovenous graft and fistula stenosis. In some cases, dexamethasone may act on various chemical and molecular signals. In some cases, dexamethasone may act on degradation of MCP-1 mRNA (the messenger RNA for monocyte chemoattractive protein-1) at dexamethasone levels from 10 nM to 1 μM In some cases, dexamethasone may result in a decrease of inflammatory protein MCP-1 expression at dexamethasone levels of 50 nM. In some cases, dexamethasone levels of 10 nM to 1 μM may decrease TNFα levels. In some cases, dexamethasone levels of 10 nM to 1 μM may increase MCP-1 and decrease the number of dexamethasone binding sites and binding affinity in cells, resulting in increased inflammation. In some cases, dexamethasone may increase IL-10 levels at dexamethasone levels of 1 nM to 100 nM, which helps to decrease MCP-1 levels and serves to increase the number of binding sites and binding affinity of dexamethasone within cells. In some cases, dexamethasone may improve endothelial cell migration, resulting in quicker healing of the vessel at dexamethasone levels of 1μM. In some cases, this may be particularly relevant in the resolution of thrombosis at the endothelial surface of a vein experiencing DVT.

Usually, dexamethasone may be supplied in numerous formulations, including but not limited to tablets, elixir, ophthalmic ointments, suspensions, solutions and as an injectable for intravenous administration. In some cases, dexamethasone may be used for a variety of clinical conditions that include the treatment of asthma, cerebral edema, arthritis, ocular and dermatological conditions. In some cases, dexamethasone may be delivered to accomplish localized effect by soft tissue infiltration, intra-articular injection, intra-ocular injection and intra-lesional (skin) injection with minimal side effects. Dexamethasone may be used for intra-articular or soft tissue injection and by intralesional injection. Dexamethasone has been approved for various indications, including endocrine disorders, rheumatic disorders, collagen diseases, dermatologic diseases, allergic states, ophthalmic diseases, gastrointestinal diseases, respiratory diseases, hematologic disorders, neoplastic diseases, edematous states, tuberculous meningitis, trichinosis with neurologic or myocardial involvement and diagnostic testing of adrenocortical hyperfunction. In some instances, Dexamethasone may be administered by intra-articular or soft tissue injection for synovitis of osteoarthritis, rheumatoid arthritis, acute and subacute bursitis, acute gouty arthritis, epicondylitis, acute nonspecific tenosynovitis, and post-traumatic osteoarthritis. In some instances, dexamethasone may be administered by intralesional injection: keloids, localized hypertrophic, infiltrated, inflammatory lesions of: lichen planus, psoriatic plaques, granuloma annulare, and lichen simplex chronicus (neurodermatitis), discoid lupus erythematosus, necrobiosis lipoidica diabeticorum, alopecia areata, and may also be useful in cystic tumors of an aponeurosis or tendon (ganglia). In some embodiments, dexamethasone delivered minimally invasively by catheter into the adventitia and perivascular tissue around veins that have experienced DVT and subsequently been recanalized may decrease the inflammation that could lead to rethrombosis, venous wall and valve fibrosis and stiffening and the accompanying venous reflux and hypertension. In some embodiments, one or more of these outcomes typically accompanies chronic PTS. In some embodiments, the dexamethasone delivery may improve venous patency and reduce the rate of progression to PTS. In some cases, dexamethasone may be used reduce perivascular edema or signs of perivascular inflammation in thrombosed segments of veins affected by DVT or PTS.

### Dosage and Formulation of Agents

In some cases, dexamethasone may be commercially available as Dexamethasone Sodium Phosphate Injection, USP, 4 mg/mL. In some cases, dexamethasone may be commercially available as Dexamethasone 3.3 mg/mL Solution for Injection or Dexamethasone Phosphate 4 mg/mL Solution for Injection. In some cases, Dexamethasone Sodium Phosphate Injection, USP, 4 mg/mL, comprises 4.37 mg/mL of dexamethasone sodium phosphate, which may be equivalent to 4 mg/mL of dexamethasone phosphate.

In some cases, recommended total dosages of injected Dexamethasone Sodium Phosphate for various sites is as provided in Table 4.

TABLE 4
Recommended Dexamethasone Dosage
Amount Based on Injection Site

| | Indicated Amount of |
|---|---|
| | Dexamethasone Phosphate |
| Site of Injection | (mg) |
| Large Joints (e.g., Knee) | 2 to 4 |
| Small Joints | 0.8 to 1 |
| (e.g., Interphalangeal, Temporo-mandibular) | |
| Bursae | 2 to 3 |
| Tendon Sheaths | 0.4 to 1 |
| Soft Tissue Infiltration | 2 to 6 |
| Ganglia | 1 to 2 |

In some embodiments, the local administration of agents for treatment of PTS may be at a dosage similar to that used for soft tissue infiltration. In some embodiments, the local administration of dexamethasone for treatment of PTS may be at a dosage similar to that used for soft tissue infiltration. In some embodiments, Dexamethasone Sodium Phosphate for Injection USP, 4 mg/mL, may be indicated at doses of 2-6 mg for soft tissue infiltration, which is similar to the connective tissue surrounding blood vessels.

In some embodiments, the therapeutically effective dose for treating a thrombosed vein segment ranges from about 0.1 mg per cm of thrombosed vein to about 10 mg per cm of thrombosed vein, about 0.5 mg per cm of thrombosed vein to about 5 mg per cm of thrombosed vein, about 1 mg per cm of thrombosed vein to about 5 mg per cm of thrombosed vein, or about 1 mg per cm of thrombosed vein to about 3 mg per cm of thrombosed vein. In some embodiments, the therapeutically effective dose for treating a thrombosed vein segment is 1.28 mg per cm of thrombosed vein. In some embodiments, the therapeutically effective dose for treating a thrombosed vein segment is 3.84 mg for 3 cm of thrombosed vein. In some embodiments, the therapeutically effective dose for treating a thrombosed vein segment is at least about 0.01 mg, 0.02 mg, 0.03 mg, 0.04 mg, 0.05 mg, 0.06 mg, 0.07 mg, 0.08 mg, 0.09 mg, 0.1 mg, 0.2 mg, 0.3 mg, 0.4 mg, 0.5 mg, 0.6 mg, 0.7 mg, 0.8 mg, 0.9 mg, 1 mg, 2 mg, 3 mg, 5 mg, 5 mg, 6 mg, 7 mg, 8 mg, 9 mg, or 10 mg per cm of thrombosed vein. In some embodiments, the therapeutically effective dose for treating a thrombosed vein segment is no more than about 0.1 mg, 0.2 mg, 0.3 mg, 0.4 mg, 0.5 mg, 0.6 mg, 0.7 mg, 0.8 mg, 0.9 mg, 1 mg, 2 mg, 3 mg, 5 mg, 5 mg, 6 mg, 7 mg, 8 mg, 9 mg, 10 mg, 15 mg, 20 mg, 25 mg, 30 mg, 35 mg, 40 mg, 45mg, or 50 mg per cm of thrombosed vein. In some embodiments, the total therapeutically effective dose for treating a thrombosed vein segment is at least about 0.01 mg, 0.05 mg, 0.1 mg, 0.2 mg, 0.3 mg, 0.4 mg, 0.5 mg, 0.6 mg, 0.7 mg, 0.8 mg, 0.9 mg, 1 mg, 2mg, 3 mg, 5 mg, 5 mg, 6 mg, 7 mg, 8 mg, 9 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, or 100 mg. In some embodiments, the total therapeutically effective dose for treating a thrombosed vein segment is no more than about 0.1 mg, 0.5 mg, 1 mg, 2mg, 3 mg, 5 mg, 5 mg, 6 mg, 7 mg, 8 mg, 9 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, or 100 mg. In some embodiments, the delivered agents reduce inflammation. In some embodiments, the delivered agents reduce clotting and thrombosis. In some embodiments, the delivered therapeutic agents comprise dexamethasone. In some embodiments, the delivered therapeutic agents comprise tPA.

In some embodiments, the thrombosed vein segment length treated by the therapeutically effective dose ranges from about 1 cm to about 80 cm, about 5 cm to about 50 cm, about 1 cm to about 40 cm, about 1 cm to about 30 cm, about 1 cm to about 20 cm, about 1 cm to about 10 cm, about 10 cm to about 20 cm, about 10 cm to about 80 cm, or about 20 cm to about 80 cm. In some embodiments, the thrombosed vein segment length treated by the therapeutically effective dose is at least about 0.5 cm, 1 cm, 5 cm, 10 cm, 15 cm, 20 cm, 25 cm, 30 cm, 35 cm, 40 cm, 45 cm, 50 cm, 55 cm, 60 cm, 65 cm, 70 cm, 75 cm, or 80 cm. In some embodiments, the thrombosed vein segment length treated by the therapeutically effective dose is no more than about 5 cm, 10 cm, 15 cm, 20 cm, 25 cm, 30 cm, 35 cm, 40 cm, 45 cm, 50 cm, 55 cm, 60 cm, 65 cm, 70 cm, 75 cm, or 80 cm. In some embodiments, the maximum thrombosed vein segment length treated by the therapeutically effective dose is 50 cm, and the maximum dose of dexamethasone to be delivered is about 64 mg at a prescribed dosage of 1.28 mg/cm of thrombosed vein. In some embodiments, the maximum thrombosed vein segment length treated by the therapeutically effective dose is 80 cm, and the maximum dose of dexamethasone to be delivered is about 100 mg at a prescribed dosage of 1.25 mg/cm of thrombosed vein. In some embodiments, the delivered agents reduce inflammation. In some embodiments, the delivered agents reduce clotting and thrombosis. In some embodiments, the delivered therapeutic agents comprise dexamethasone. In some embodiments, the delivered therapeutic agents comprise tPA.

In some embodiments, the therapeutically effective concentration of glucocorticoid that is delivered into perivenous tissue may range from about 0.01 mg/ml to about 100 mg/ml, about 0.01 to about 50 mg/ml, about 0.1 mg/ml to about 50 mg/ml, about 0.1 mg/ml to about 10 mg/ml, about 0.5 mg/ml to about 5 mg/ml, or about 1 mg/ml to about 10 mg/ml. In some embodiments, the therapeutically effective concentration of glucocorticoid that is delivered into perivenous tissue may be at least about 0.01 mg/ml, 0.02 mg/ml, 0.03 mg/ml, 0.04 mg/ml, 0.05 mg/ml, 0.06 mg/ml, 0.07 mg/ml, 0.08 mg/ml, 0.09 mg/ml, 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/m, 1.0 mg/ml, or 5 mg/ml. In some embodiments, the therapeutically effective concentration of glucocorticoid that is delivered into perivenous tissue may be no more than 0.1 mg/ml, 0.5 mg/ml, 1.0 mg/ml, 2 mg/ml, 3 mg/ml, 4 mg/ml, 5 mg/ml, 6 mg/ml, 7 mg/ml, 8 mg/ml, 9 mg/ml, 10 mg/ml, 20 mg/ml, 30 mg/ml, 40 mg/ml, 50 mg/ml, 60 mg/ml, 70 mg/ml, 80 mg/ml, 90 mg/ml, or 100 mg/ml. In some embodiments, the therapeutically effective concentration of glucocorticoid that is delivered into perivenous tissue may range from about 0.1 mg/ml to about 10 mg/ml.

In some embodiments, the therapeutically effective concentration of dexamethasone that is delivered into perivenous tissue may range from about 0.01 mg/ml to about 100 mg/ml, about 0.01 to about 50 mg/ml, about 0.1 mg/ml to about 50 mg/ml, about 0.1 mg/ml to about 10 mg/ml, about 0.5 mg/ml to about 5 mg/ml, or about 1 mg/ml to about 10 mg/ml. In some embodiments, the therapeutically effective concentration of dexamethasone that is delivered into perivenous tissue may be at least about 0.01 mg/ml, 0.02 mg/ml, 0.03 mg/ml, 0.04 mg/ml, 0.05 mg/ml, 0.06 mg/ml, 0.07 mg/ml, 0.08 mg/ml, 0.09 mg/ml, 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/m, 1.0 mg/ml, or 5 mg/ml. In some embodiments, the therapeutically effective concentration of dexamethasone that is delivered into perivenous tissue may be no more than 0.1 mg/ml, 0.5 mg/ml, 1.0 mg/ml, 2 mg/ml, 3 mg/ml, 4 mg/ml, 5 mg/ml, 6 mg/ml, 7 mg/ml, 8 mg/ml, 9 mg/ml, 10 mg/ml, 20 mg/ml, 30 mg/ml, 40 mg/ml, 50 mg/ml, 60 mg/ml, 70 mg/ml, 80 mg/ml, 90 mg/ml, or 100 mg/ml. In some embodiments, the therapeutically effective concentration of dexamethasone that is delivered into perivenous tissue may range from about 0.1 mg/ml to about 10 mg/ml. In some embodiments, the therapeutically effective concentration of dexamethasone that is delivered into perivenous tissue may be about 3.2 mg/ml. In some embodiments, the therapeutically effective concentration of dexamethasone that is delivered into perivenous tissue may be about 3 mg/ml. In some embodiments, the therapeutically effective concentration of dexamethasone that is delivered into perivenous tissue may be about 2 mg/ml. In some embodiments, the therapeutically effective concentration of dexamethasone that is delivered into perivenous tissue may be about 1.6 mg/ml. In some embodiments, the therapeutically effective concentration of dexamethasone that is delivered into perivenous tissue may be about 8 mg/ml. In some embodiments, the therapeutically effective concentration of dexamethasone that is delivered into perivenous tissue may be about 10 mg/ml.

In some embodiments, the volume of therapeutic agent that is delivered into perivenous tissue may range from about 0.01 ml per cm of thrombosed vein to about 100 ml per cm of thrombosed vein, about 0.01 to about 50 ml per cm of thrombosed vein, about 0.1 ml to about 50 ml per cm of thrombosed vein, about 0.1 ml to about 10 ml per cm of thrombosed vein, about 0.5 ml to about 5 ml per cm of thrombosed vein, or about 1 ml to about 10 ml per cm of thrombosed vein. In some embodiments, the volume of therapeutic agent that is delivered into perivenous tissue is at least about 0.01 ml, 0.02 ml, 0.03 ml, 0.04 ml, 0.05 ml, 0.06 ml, 0.07 ml, 0.08 ml, 0.09 ml, 0.1 ml, 0.2 ml, 0.3 ml, 0.4 ml, 0.5 ml, 0.6 ml, 0.7 ml, 0.8 ml, 0.9 mg/m, 1 ml, 2 ml, 3 ml, 4 ml, 5 ml, 6 ml, 7 ml, 8 ml, 9 ml, or 10 ml per cm of thrombosed vein. In some embodiments, the volume of therapeutic agent that is delivered into perivenous tissue is no more than about 0.1 ml, 0.2 ml, 0.3 ml, 0.4 ml, 0.5 ml, 0.6 ml, 0.7 ml, 0.8 ml, 0.9 mg/m, 1 ml, 2 ml, 3 ml, 4 ml, 5 ml, 6 ml, 7 ml, 8 ml, 9 ml, 10 ml, 15 ml, 20 ml, or 25 ml per cm of thrombosed vein. In some embodiments, the volume of therapeutic agent that is delivered into perivenous tissue may range from about 0.5 ml per cm of thrombosed vein to about 3 ml per cm of thrombosed vein. In some embodiments, the delivered therapeutic agents reduce inflammation. In some embodiments, the delivered therapeutic agents reduce clotting and thrombosis. In some embodiments, the delivered therapeutic agents comprise dexamethasone. In some embodiments, the delivered therapeutic agents comprise tPA.

In some embodiments, the therapeutically effective dosage of one or more agents for treating a thrombosed vein segment ranges from about 0.1 to 10 mL of volume per cm of affected tissue. In some embodiments, about 1 to about 10 mg/mL dexamethasone may be delivered in doses of 0.5 to 3 mL volume per cm of target vein length. In some embodiments, the therapeutically effective dose for treating a thrombosed vein segment is 1.28 mg per cm of thrombosed vein. In some embodiments, the therapeutically effective dose for treating a thrombosed vein segment is 3.84 mg for 3 cm of thrombosed vein. In some embodiments, the entire segment of vein can be treated by moving the delivery device around and targeting the needle for delivery through the vein wall in different segments where thick, adherent clot is not present. In some embodiments, where thick, adherent clot or organized thrombus may be present, tPA or other fibrinolytic therapies may be delivered directly into the organized tissue. In some embodiments, the direct delivery of a fibrinolytic agent may aid with the resolution of the thrombus.

Multiple injections may be typically needed to treat a segment of vein longer than a few centimeters. In some embodiments, an injection may be tracked with a contrast agent to confirm distribution around the target vein segment. In some embodiments, an injection may be up to 8 mL volume but will typically be in the 1-3 mL range prior to moving to the next injection site along the length of the vein. In some embodiments, injection sites can be chosen based on distribution pattern in order to provide optimal coverage of the treatment site. In some embodiments, one administration may comprise at least 1 injection, 2 injections, 3 injections, 4 injections, 5 injections, 6 injections, 7 injections, 8 injections, 9 injections, 10 injections, 15 injections, 20 injections, or 25 injections. In some embodiments, one administration may comprise no more than 5 injections, 6 injections, 7 injections, 8 injections, 9 injections, 10 injections, 15 injections, 20 injections, or 25 injections. In some embodiments, one injection is distance apart from another injection by at least 1 cm, 2 cm, 3 cm, 4 cm, 5 cm, 6 cm, 7 cm, 8 cm, 9 cm, or 10 cm along the vein.

In some embodiments, the therapeutically effective concentration refers to a concentration that has one or more of the following effects: reduces local inflammation at or near the thrombosis, reduces the local tissue level of one or more biomarkers of inflammation, reduces the systemic level of one or more biomarkers of inflammation, reduces the local tissue level of one or more biomarkers of thrombosis, reduces the systemic level of one or more biomarkers of thrombosis, reduces indicators of PTS, reduces indicators of DVT. In some embodiments, the therapeutically effective concentration refers to a concentration that reduces local inflammation at or near the thrombosis by at least about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50%. In some embodiments, the therapeutically effective concentration refers to a concentration that reduces the local tissue level of one or more biomarkers of inflammation by at least about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50%. In some embodiments, the therapeutically effective concentration refers to a concentration that reduces the systemic level of one or more biomarkers of inflammation by at least about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50%. In some embodiments, the therapeutically effective concentration refers to a concentration that reduces the local tissue level of one or more biomarkers of thrombosis by at least about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50%. In some embodiments, the therapeutically effective concentration refers to a concentration that reduces the systemic level of one or more biomarkers of thrombosis by at least about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50%. In some embodiments, the therapeutically effective concentration refers to a concentration that reduces indicators of PTS by at least about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50%. In some embodiments, the therapeutically effective concentration refers to a concentration that reduces indicators of DVT by at least about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50%.

In some embodiments, compositions disclosed herein may reduce indicators of PTS. In some embodiments, the indicators of PTS that may be assessed include but are not limited pain, cramps, heaviness, pruritus, paresthesia, edema, skin induration, hyperpigmentation, venous ectasia, redness, and pain during calf compression. In some embodiments, the indicators of PTS that may be assessed Villalta score or a VCSS score. In some embodiments, the indicators of PTS that may include measuring level of one or more inflammatory biomarkers after the delivery of a therapeutic composition into a perivascular tissue at or near the thrombosed segment. In some embodiments, the one or more inflammatory biomarkers comprises one or more of IL-1β, IL-2, IL-6, IL-8, IL-10, IFN-α, IFN-γ, ICAM-1, TNF-α, CRP, D-dimer, fibrinogen, MCP-1, IL-1Ra, IL-1α, MMP-1, MMP-2, MMP-8, MMP-9, TIMP, ICAM-1, VCAM-1, and soluble P-selectin. In some embodiments, the indicators of PTS that may include assessing the patency of a thrombosed segment, a decrease or a lack of increase in rethrombosis in the thrombosed segment, a decrease or a lack of increase in venous reflux, and/or a decrease or a lack of increase in fibrosis and stiffness of wall and valve of the vein affected by DVT. The compositions disclosed herein may maintain or increase the patency of a thrombosed segment for at least 5 weeks, 3 months, 6 months, 12 months, 18 months, 24 months or more. Alternatively or in combination, the compositions of the invention may result in a decrease or a lack of increase in rethrombosis for at least 5 weeks, 3 months, 6 months, 12 months, 18 months, 24 months or more. Alternatively or in combination, the compositions of the invention may result in a decrease or a lack of increase in venous reflux for at least 5 weeks, 3 months, 6 months, 12 months, 18 months, 24 months or more. Again, alternatively or in combination, the compositions of the invention may result in a decrease or a lack of increase in fibrosis and stiffness of the wall and/or valve of the vein for at least 5 weeks, 3 months, 6 months, 12 months, 18 months, 24 months or more. Rethrombosis, venous reflux and/or fibrosis and stiffness of the wall and/or valve (or lack thereof) may be measured by ultrasound as described herein. In some embodiments, the perivascular edema or tissue constituent fluids may be assessed using MRI, CT, FDG-PET, ultrasound, or other non-invasive imaging modalities.

In some embodiments, a 4 mg/mL stock solution of dexamethasone may be diluted to 3.2 mg/mL with a contrast medium having at least 300 mg unbound iodine per ml. In some embodiments, each 0.4 ml of infusion may treat one cm of vessel segment in target vessels at 3.2 mg/ml of dexamethasone concentration as based on experimental data. In some embodiments, each milliliter of the stock solution of dexamethasone has 4.37 mg of dexamethasone sodium phosphate equivalent to 4 mg of dexamethasone phosphate or 3.33 mg of dexamethasone. In some embodiments, the stock solution of dexamethasone may be diluted by 20% prior to administration with a contrast medium to enhance visualization of the injection field under X-ray fluoroscopy. In some embodiments, the diluted solution of dexamethasone may have a final concentration of 3.2 mg dexamethasone phosphate (3.5 mg dexamethasone sodium phosphate, or 2.67 mg dexamethasone) in each milliliter of solution. In some embodiments, the dexamethasone at a concentration of 3.2 mg/mL may result in 0.4 ml being delivered per centimeter of thrombosed vein. In some embodiments, allotting for an additional 25% (16 mg) due to variability in anatomy, distribution pattern and intravascular diagnostic loss, a total dose of 80 mg, or 20 mL of dexamethasone sodium phosphate injection, USP (4 mg/mL) combined with 5 mL of contrast, may be provided for each procedure. In some embodiments, the intended dosage may be limited to 64 mg (20 mL at 3.2 mg/mL), which is well under approved systemic exposure (300 mg in a 50 kg individual) for dexamethasone. In some embodiments, Dexamethasone Sodium Phosphate Injection USP, 4 mg/mL label may indicate a systemic dosing of up to 6 mg/kg IV bolus for the treatment of shock. In some embodiments, multiple doses may be needed to treat longer diseased regions. In some embodiments, the dexamethasone solution may be diluted with saline or water for injection in order to provide a solution with lower concentration but greater infusion volume per cm of target vessel. In some embodiments, the solution may comprise at least 1 mg/mL dexamethasone, optionally around 20% contrast medium with at least 200 mg unbound iodine per mL, and a balance of saline solution or other injectable medium, and the intended dosing may be at least 0.5 mg dexamethasone per cm of target vessel with the delivery of a volume of at least 0.5 mL per cm of target vessel length.

In some embodiments, dexamethasone has been safely administered using a local catheter-based injection into blood vessels. In some embodiments, a dosage of 10 mg per 3 cm treatment site in arteriovenous graft anastomoses was observed to have no toxic effects in preclinical porcine studies. In some embodiments, a dosage of 1.6 mg dexamethasone per cm of lesion was safely delivered by perivascular injection around revascularized femoral and popliteal arteries. In some embodiments, such delivery provides a safe procedure at this dosage per unit length, and patency compared favorably to historical data in similar patients.

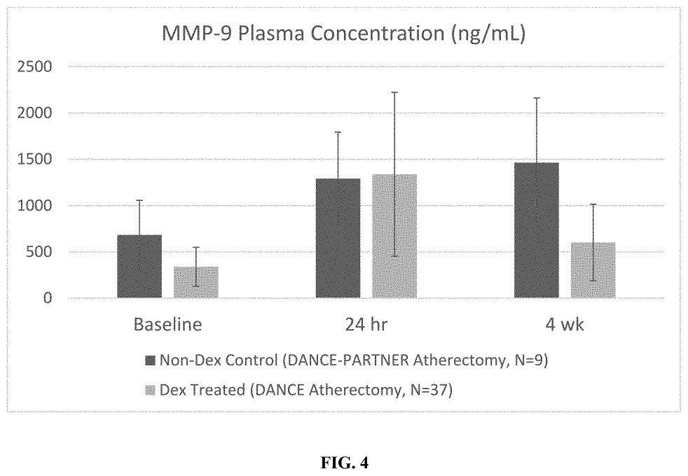

In some cases, MMP-9 may be one of the indicators of advancement toward PTS if it is present for longer time frames. In some cases, MMP-9 levels may serve as an indicator for chronic inflammation associated with PTS. In some cases, during the early course of thrombus resolution, MMP-9 may aid breaking down a clot through mediation of macrophage and collagen content of the resolving thrombus. In some cases, however, if MMP-9 remains at a high concentration, this may lead to increased stiffness of the extracellular matrix and collagen-elastin fibers, stiffening of the vein wall and leading to PTS. In some cases, direct, perivascular administration of dexamethasone may counteract the effect of MMP-9 by keeping high short-term MMP-9 levels but reducing long-term MMP-9 levels through direct or paracrine effects on the tissue. FIG. 4 shows an experimental results of MMP-9 plasma concentration over time before and after dexamethasone injection. In some cases, during a clinical trial to inject dexamethasone at 3.2 mg/mL and a dose of 0.5 mL per cm of affected vessel length into superficial femoral and popliteal arteries during revascularization procedure, MMP-9 levels were measured in circulating blood. In some cases, when MMP-9 levels were compared to a series of control subjects that did not receive dexamethasone injections, both control and local dexamethasone-treated subjects had statistically significant (p<0.05) and substantial increase in MMP-9 level from baseline (pre-procedure) to 24 hours after the procedure, but only the local dexamethasone-treated subjects (DANCE Atherectomy) patients had a statically significant and substantial decrease in MMP-9 between 24 hours and 4 weeks, nearly back to baseline levels. In some cases, local administration of dexamethasone may prevent progression to PTS in patients with venous thrombosis.

### Modified Release of Agents

In some embodiments, the one or more agents for local delivery into perivenous tissue described herein may be formulated for a modified release. In some embodiments, the one or more agents for local delivery into perivenous tissue described herein may be formulated sustained-release dosage. In some embodiments, sustained release dosage forms or controlled release dosage forms may be designed to release the one or more agents at a predetermined rate and maintain a constant concentration for a specific period of time with minimum side effects. In some embodiments, the formulation allows maintenance of drug release over a sustained period but not at a constant rate. In some embodiments, the formulation allows maintenance of drug release over a sustained period at a nearly constant rate.

In some embodiments, the modified release, such as extended release, sustained release, or controlled release, may be achieved by various formulations, including but not limited to liposomes, drug-polymer conjugates, microparticles, molecular polymerization of the drug, and nanoparticles. In some embodiments, the one or more agents described herein may be formulated into a polymeric carrier. In some embodiments, the one or more agents described herein may be embedded within a polymer. In some embodiments, the composition for local administration by the methods and devices provided herein may comprise a liquid, gel, or semisolid into the tissue. In some embodiments, the gel comprises a hydrogel. In some embodiments, long-acting injectables may include but are not limited to oil-based injections, injectable drug suspensions, injectable microspheres, and injectable in situ systems, drugs and polymers for depot injections, depot injections, polymer-based microspheres, and polymer-based in-situ forming, and injectable sustained-release drug-delivery. In some embodiments, oil-based injectable solutions and injectable drug suspensions may control the release for weeks. In some embodiments, polymer-based microspheres and in-situ gels may control the release for months

In some embodiments, the polymer comprises one or more of polylactides (PLA), polyglycolides (PGA), poly(lactide-co-glycolide) (PLGA), poly(c-caprolactone) (PCL), polyglyconate, polyanhydrides, polyorthoesters, poly(dioxanone), polyalkylcyanoacrylates, poly(ether ester urethane)s, poly(ethylene glycol) (PEG), poly(propylene glycol) (PPG), PEG-chitosan polymer, PEG copolymer, PLGA copolymer, and PPG copolymer. In some cases, the polymer is biodegradable. In some cases, the polymer is bioresorbable.

In some embodiments, the composition comprises a particle suspension. In some embodiments, the particles are less than 1 micron in size. In other embodiments, the particles are between 1 and 100 microns in size. In other embodiments, the particles are larger than 100 microns. In some embodiments, the particles are spherical. In some embodiments the particles are ellipsoid, rod-like, disc-like or other shapes. In some embodiments, all of the particles are approximately the same size, or monodisperse. In some embodiments, the particles are a range of sizes, or polydisperse. In general, the size, shape and physical properties of the particles may be selected to optimize the desired properties of the final product, including injectability, diffusion, physical stability, biodistribution, response to the composition, and ease of manufacturing. With respect to injectability, relatively smaller particles may be more desirable for injection through a needle.

In some embodiments, the sustained release of the agent at the injection site may last for at least 1 day, 2 day, 3 days, 4 days, 5 days, 6 days, 1 week, 2 weeks, 3 weeks, 4 weeks, 1 month, 2 months, 3 months, 4 months, 5 months, or 6 months. In some embodiments, the sustained release of the agent at the injection site may last for no more than 1 day, 2 day, 3 days, 4 days, 5 days, 6 days, 1 week, 2 weeks, 3 weeks, 4 weeks, 1 month, 2 months, 3 months, 4 months, 5 months, or 6 months.

In some embodiments, a long-acting injectable formulation to a local tissue injection site may provide many advantages when compared with conventional formulations of the same agent or a systemic administration of the same agent. In some embodiments, the advantages include but are not limited to: a predictable drug-release profile during a defined period of time following each injection; better patient compliance; ease of application; improved systemic availability by avoidance of first-pass metabolism; reduced dosing frequency (i.e., fewer injections) without compromising the effectiveness of the treatment; decreased incidence of side effects; and overall cost reduction of medical care.

### Systems and Methods for Localized Administration

Provided herein are medical instruments and medical methods for localized drug delivery to a patient's tissue. The medical instrument can comprise a catheter shaft assembly having at least an injection lumen and an inflation lumen, an inflatable component (e.g., a balloon) at a distal end of the catheter shaft assembly and in fluid communication to the inflation lumen, a tissue penetrating member (e.g., a needle) coupled to the inflatable component and in fluid communication to the injection lumen, fluid routing pathways between the catheter shaft assembly and the inflatable component and between the catheter shaft assembly and the tissue penetrating member, and at least one protective element coupled to the inflatable component in proximity to the tissue penetrating member. The catheter shaft assembly can be inserted into and advanced within a body lumen of a patient over a guidewire to a predetermined position within the body lumen when the inflatable component is in a contracted configuration. The inflatable component can then be inflated by hydraulic fluid, which is supplied through the inflation lumen, into an expanded configuration such that the tissue penetrating member is exposed. The tissue penetrating member, which is in fluidic communication with a drug lumen, can penetrate the body lumen and deliver a drug into the patient's tissue. The inflatable component can be deflated upon a completion of the drug delivery, such that the catheter shaft assembly can be further advanced in or retracted from the body lumen. The inflatable component and a fluid communication line from the injection lumen to the tissue penetrating element can be kept separate and sealed off from each other using fluid routing techniques between the catheter shaft assembly and the inflatable component. The body lumen can comprise a vein of a patient. An exemplary medical instruments and medical methods for localized drug delivery to a patient's tissue as described in U.S. patent application Ser. No. 16/977,355, filed on Sep. 1, 2020, which is incorporated herein by reference.

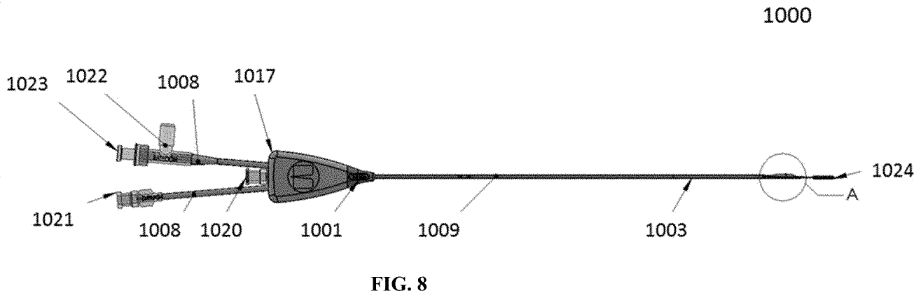

FIG. 8 is a schematic, perspective view of a medical instrument 1000 for localized drug delivery in accordance with some embodiments of the disclosure. The medical instrument 1000 can comprise a catheter shaft assembly 1009 and a hub 1017 coupled to a proximal end of the catheter shaft assembly 1009. A labeling 1001 can be provided to the medical instrument to show particular information for the medical instrument, such as the working diameter of patient body lumens that the medical instrument can treat. The labeling 1001 can be provided at any appropriate position of the medical instrument, for example at the hub or at an injunction of the hub and the catheter shaft assembly.

The catheter shaft assembly 1009 can be provided as a micro-fabricated intraluminal catheter. The catheter shaft assembly can include a catheter body tubing. In some embodiments, the catheter body tubing can be provided with a diameter of 1 mm to 3 mm and a length of 50 cm to 180 cm. One or more lumens (e.g., fluid transmission channels) can be accommodated within the catheter body tubing, which one or more lumens each has a longitudinal axis parallel to a longitudinal axis of the catheter body tubing. The one or more lumens can include at least one of an injection lumen, in inflation lumen, or a guidewire lumen. The injection lumen can be provided to transmit a drug or agent to be delivered to the patient. The inflation lumen can be provided to transmit a fluid to inflate an inflatable component (e.g., a balloon). The guidewire lumen can be provided through which a guidewire can be extended. In some embodiments where a guidewire lumen is not provided within the catheter shaft assembly, a stiffening element 1024 can be provided at the distal end of the catheter shaft assembly.

In some embodiments, the catheter shaft assembly can additionally include a torque transmission tube 1003 with its axis parallel to the axis of the catheter body tubing. The torque transmission tube can be provided to transmit a torque from the proximal end (e.g., the user end) of the catheter shaft assembly to the distal end (e.g., the working end) of the catheter shaft assembly. The torque transmission tube may be comprised of a stainless steel hypodermic tubing that is cut in a pattern to allow the transmission of torque while removing the bending stiffness of the tube. An exemplary cut pattern is a spiral cut or a broken spiral cut as described in U.S. Pat. No. 7,708,704, the full disclosures of which is incorporated herein by reference.

The hub 1017 can be coupled to the proximal end of the catheter shaft assembly 1009 and comprise one or more interfaces/ports which are in fluidic communication with the one or more lumens of the catheter body tubing. The one or more interfaces/ports can be coupled to the one or more lumens of the catheter body tubing via a tube such as tube 1008. In some embodiments, the hub can comprise an injection port 1021 coupled to the injection lumen, an inflation port 1023 coupled to the inflation lumen, and a guidewire port 1020 coupled to the guidewire lumen of the catheter body tubing. In an example where the medical instrument has a scope-compatible configuration, the injection port 1021 and the inflation port 1023 can be provided. In another example, where the medical instrument has a guidewire-compatible configuration, the guidewire port 1020 can be additionally provided. The one or more tubes can be coupled with the catheter body tubing by adhesive bonding, potting, thermal fusing, or over-molding, for example. The one or more interfaces/ports of the hub can be Luer interfaces or handles with which a user can interact with the medical instrument to provide or remove a hydraulic fluid, guidewire and drug(s) into or from the medical instrument. For instance, the hydraulic fluid can be supplied into the inflatable component via the inflation lumen using a syringe. In some embodiments, the one or more interfaces/ports of the hub can each be provided with a pressure governor to regulate a pressure of the fluid transmitted via the interface/port. For instance, a pressure governor 102 can be provided to the inflation port 1023. The pressure governor 1022 can be a pressure relief valve with spring-loaded silicone stopper against a valve seat. The pressure governor 1022 can be configured to regulate a pressure of the hydraulic fluid supplied to the inflatable component.

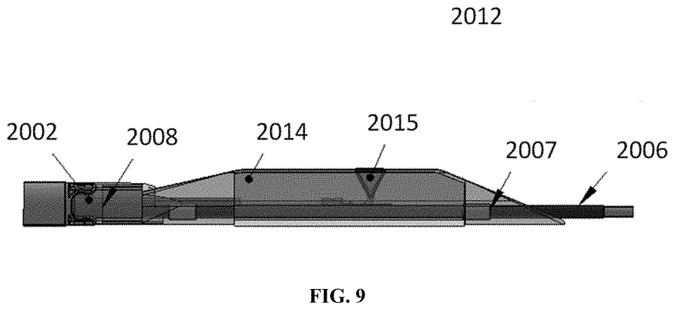

The inflatable component can be provided at a distal end of the catheter shaft assembly. FIG. 9 is an enlarged view showing portion A of FIG. 8 where the inflatable component is positioned. In some embodiments, the inflatable component can comprise an inflatable body 2012 and a protective element 2015 provided at the inflatable body 2012. The inflatable body 2012 can be coupled to the inflation lumen by various coupling member, such as an adhesive 2007. The protective element 2015 can be provided to prevent any damage of the inflatable body during an inflation process.

The inflatable body 2012 can be a hydraulic actuating balloon which is inflatable when a hydraulic fluid is provided into the hydraulic actuating balloon. For instance, the hydraulic actuating balloon can be made from an elastic material. The hydraulic fluid can be a compressed air or liquid. In some embodiments, the inflatable body 2012 can include a first section and a second section which are inflated and deployed sequentially and/or successively. For instance, the first section of the inflatable body can be inflated and/or deployed at a first pressure, and the second section of the inflatable body can then be inflated and/or deployed at a second pressure which is higher than the first pressure. The second section may not be inflated during an inflation of the first section. The first section may not be further inflated during an inflation of the second section. In some instances, the first pressure and the second pressure can be successive inflation pressures. The sequential inflation can be effected by providing the first section and the second section with different elasticities. Similar inflatable bodies with multiple layers and methods for manufacturing such layers are described in U.S. patent applications Ser. No. 11/858,797 (U.S. Pat. No. 7,691,080), Ser. No. 12/711,141 (U.S. Pat. No. 8,016,786), Ser. No. 13/222,977 (U.S. Pat. No. 8,721,590), Ser. No. 14/063,604 (U.S. Pat. No. 9,789,276), and Ser. No. 15/691,138, the contents of which are fully incorporated herein by reference.

A material of the inflatable body 2012 can allow the inflatable body to be inflated/converted from a lower profile to a larger profile once an inflation pressure is applied to the inflatable body, such that a size of the inflatable body can be increased. The inflatable body can be made of a thin, semi-flexible but relatively non-distensible material, such as a polymer, for instance, Parylene (types C, D, F or N), silicone, polyurethane, Nylon, Pebax or polyimide. The inflatable body can return substantially to its original configuration and orientation (e.g., the unactuated/uninflated condition) when the hydraulic fluid is removed. The inflatable body can be capable of withstanding pressures of up to about 300 psi upon application of the hydraulic fluid.

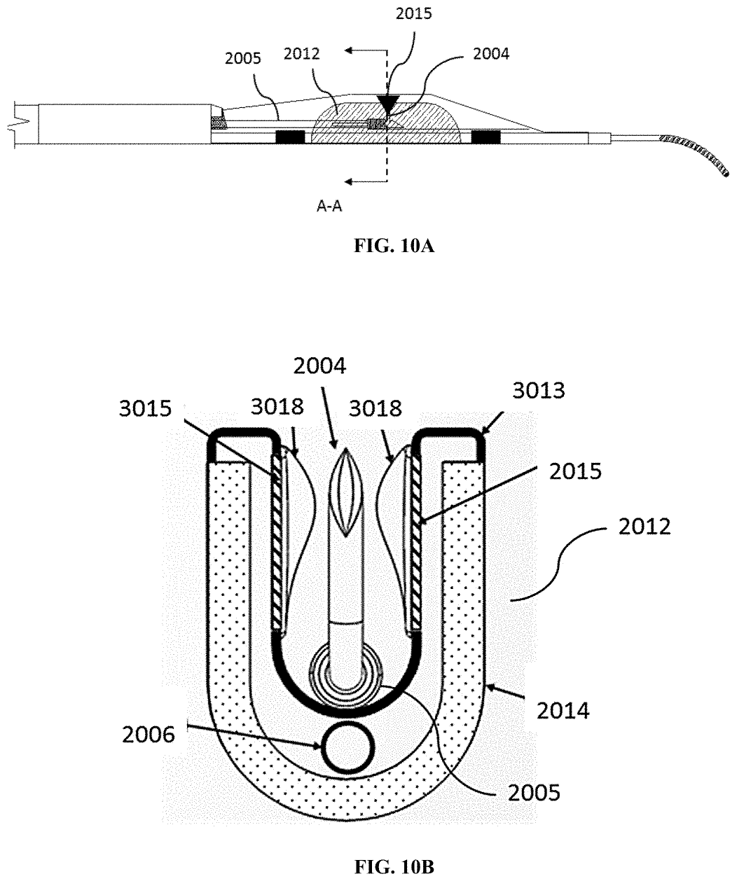

As shown in FIG. 10A and FIG. 10B, at least one tissue penetrating member 2004 can be coupled to the inflatable body 2012 in an orientation transverse to the longitudinal axis of the catheter shaft assembly 1009. The tissue penetrating member 2004 can be a needle which is configured to penetrate into a luminal wall and/or deliver a drug into the luminal wall. The tissue penetrating member 2004 can be another structure such as an atherectomy blade, an optical fiber for delivering laser energy, a mechanical abrasion, or a drilling component, to name a few examples. In some embodiments, the tissue penetrating member can comprise at least one needle or microneedle.

The tissue penetrating member can be in fluidic communication with a flexible drug line tubing 2005. The flexible drug line tubing 2005 can be a separate tubing piece which is received in the injection lumen of the catheter shaft assembly 1009 and in fluidic communication with the injection port at the hub, such that a pharmaceutical agent or a diagnostic agent can be transmitted from the injection port 1021 to the tissue penetrating member along the flexible drug line tubing 2005. Alternatively or in combination, a proximal end of the flexible drug line tubing 2005 can be coupled to an outlet of the injection lumen of the catheter shaft assembly 1009. The flexible drug line tubing 2005 can be made of an appropriate material which exhibits a flexibility or shape memory property. A distal end of the flexible drug line tubing 2005 proximal to the location that the tissue penetrating member bends upright can be in fluidic communication to the tissue penetrating member and can be affixed to an exterior surface of the inflatable body 2012. The distal end of the flexible drug line tubing can be affixed to the exterior surface of the inflatable body 2012 by an adhesive, such as cyanoacrylate.

In some instances, the flexible drug line tubing can be routed through the wall of the inflatable body by passing through a junction of elastomeric material coated with parylene. The flexible drug line tubing can be provided within the inflatable body and routed through the inflatable body at the distal end of the flexible drug line tubing. A junction of elastomeric material coated with parylene can be provided at the inflatable body where the flexible drug line tubing passes from the interior of the inflatable body to the exterior of the inflatable body, such that the flexible drug line tubing is sealed against the inflatable body at the junction.

The medical instrument shown in FIG. 10A has an involuted contracted configuration where the tissue penetrating member (e.g., a needle) is not deployed/exposed. The catheter shaft assembly, in use, can be inserted in and advanced along the patient's body lumen in this involuted contracted configuration until it reaches a target region within the body lumen. FIG. 10B is a cross-sectional view along line A-A of FIG. 10A. As shown in FIG. 10B, the inflatable body 2012 can include a first section 3013 and a second section 2014. In some embodiments, the first section 3013 can be an elastic membrane having a first elasticity, and the second section 2014 can be a rigid polymer (e.g., parylene) having a second elasticity which is less than the first elasticity, such that the first section and the second section can be successively inflated. Here, the parameter elasticity means the ability of a body to resist a distorting influence and to return to its original size and shape when that influence or force is removed. An object having a smaller elasticity can be more rigid and can be inflated under greater pressure. Alternatively, the object having smaller elasticity may be more stretchy than the object with greater elasticity, but the object with greater elasticity (e.g., the first section 3013) may undergo bending stress to open the expandable member from the involuted configuration without further stretching, while the object with less elasticity (e.g., the second section 2014) may secondarily stretch after the expandable cavity has formed a roughly circular cross-sectional shape, thus expanding the pressurized component's diameter as pressure is increased.

In the involuted contracted configuration shown in FIGS. 10A and 10B, the inflatable body 2012 can have a substantially U-shaped cross-section. The tissue penetrating member 2004 such as a needle can be coupled to the second section 3013 of the inflatable body 2012 in an orientation transverse to the longitudinal axis of the catheter shaft assembly. The tissue penetrating member 2004 can be further coupled to the injection lumen of the catheter shaft assembly via the flexible drug line tubing 2005. In the involuted contracted configuration, the needle can be coupled to the second section of the inflatable component with the needle tip pointing outwardly of the inflatable component and enclosed within walls of the inflatable component. As shown in FIG. 10B, the needle can extend approximately perpendicularly from the exterior surface of the second section of the inflatable body. Therefore, once actuated, the needle can move substantially perpendicularly to the longitudinal axis of the catheter shaft assembly and/or the injection lumen into which the flexible drug line tubing is coupled, to allow direct puncture or breach of lumen walls.

The needle can include the sharp needle tip and a needle shaft. The needle tip can provide an insertion edge or point. The needle shaft can be hollow and in fluidic communication with the distal end of the flexible drug line tubing. The needle tip can have an outlet port, permitting an injection of a pharmaceutical or drug into the patient. The needle, however, may not need to be hollow, as it may be configured like a neural probe or electrode to accomplish other tasks. The needle can be a 27-gauge, or smaller, steel needle. The needle can have a penetration length of between 0.4 mm and 4 mm.

At least one protective element 2015 can be coupled to the second section of the inflatable body 2012 at a position in proximity to the tissue penetrating member (e.g., a needle). The least one protective element 2015 can be configured such that at least the tip end of the needle can be bordered by the at least one protective element 2015 at least when the inflatable body is in the involuted contracted configuration. As shown in the cross-sectional view of FIG. 10B, at least one protective element 2015 can be provided at each lateral side of the needle, such that the needle is sheathed and protected by the protective element when the inflatable body is in the involuted contracted configuration. For instance, the protective element can be placed to surround to the sharp needle tip and function to protect the inflatable body from needle tip penetration or damage during transit of the medical instrument into and out of the body lumen.

The protective element 2015 can be integrated into an exterior wall of the second section of the inflatable body 2012. The protective element can be encapsulated by, for example parylene, and can additionally be covered by a soft adhesive 3018 such as silicone, as shown in the cross-sectional view of FIG. 10B. In some embodiments, the protective elements can be built directly into the exterior wall of the inflatable body 2012 by coating them with silicone adhesive, adhering them to a dissolvable substrate, coating the substrate with parylene, and dissolving the substrate. In this way, the protective elements and surrounding silicone can be integrated with the parylene coating and remain permanently intact to the exterior wall of the inflatable body.

The protective elements can be comprised of a hard polymer or metal. The protective elements can be made of, for example, stainless steel, platinum alloy, iridium, tungsten, gold, or the like. The protective elements can be radio-opaque to provide feedback on X-ray imaging of the catheter shaft assembly. The protective elements can be provided with a specific pattern/shape to provide an indication on an inflation status of the inflatable body. For instance, as shown in FIG. 10A, the protective elements can be provided to have an isosceles triangle shape with the vertex pointing downwards when the inflatable body is in the involuted contracted configuration. With the aid of X-ray imaging, an operator of the medical instrument can determine that the inflatable body is in the involuted contracted configuration and/or another specific configuration (e.g., a partially inflated configuration, as will be discussed below) when the protective elements is in the specific shape of an isosceles triangle shape with the vertex pointing downwards. The operator of the medical instrument can otherwise determine that the inflatable body is in a different configuration when the shape of the protective elements is changed (e.g., a fully inflated configuration).

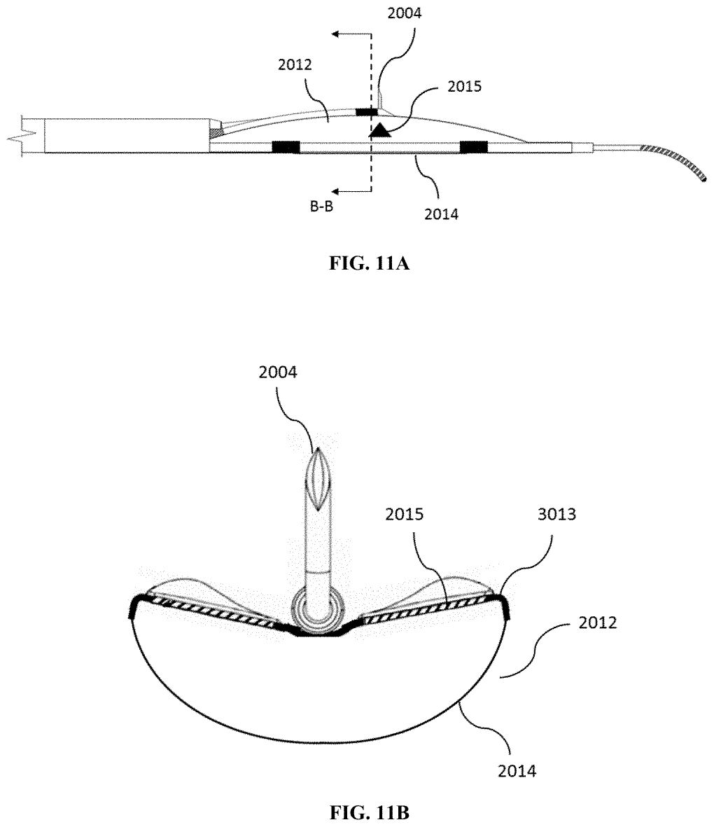

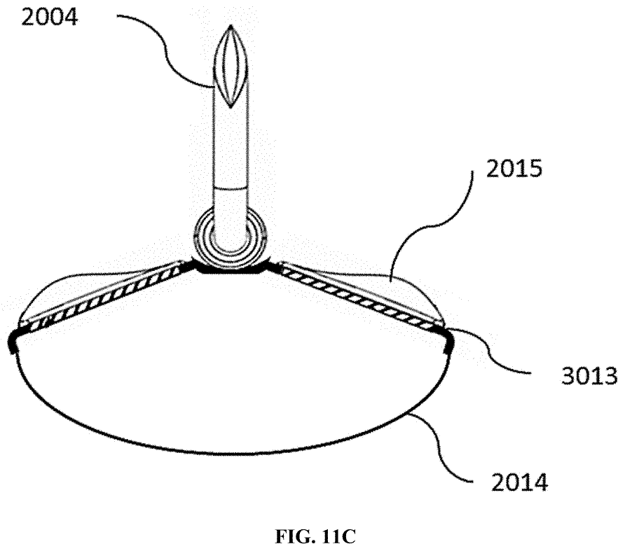

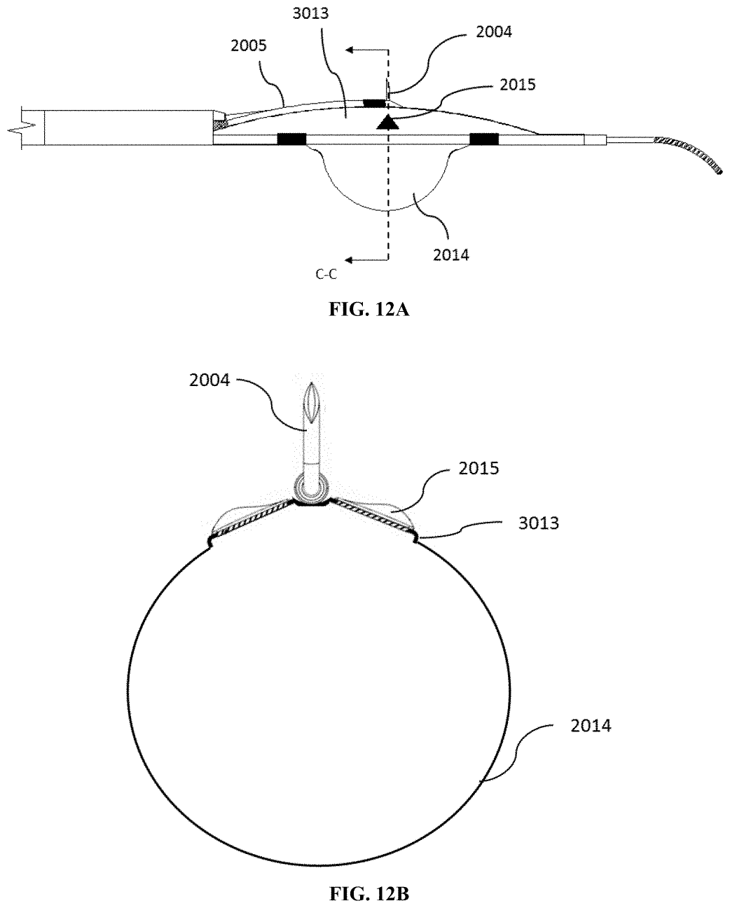

FIG. 11A shows the medical instrument for localized drug delivery where an inflatable body is at a partially inflated configuration. FIG. 11B is a cross-sectional view along line B-B of FIG. 11A, showing a transitional configuration toward the partially inflated configuration of inflatable body. FIG. 11C is a cross-sectional view along line B-B of FIG. 11A, showing a partially inflated configuration of inflatable body. FIG. 12A shows the medical instrument for localized drug delivery where the inflatable body is at a fully inflated configuration and the tissue penetrating member is deployed. FIG. 12B is a cross-sectional view along line C-C of FIG. 12A.

The inflatable body 2012 shown in FIGS. 11A and 11C has a first expanded configuration where the inflatable body is partially inflated by the hydraulic pressure which is built up in the inflatable body. The hydraulic pressure can be generated by the inflation/hydraulic fluid which is supplied into the inflatable body through the inflation lumen. In some embodiments, the first section 3013 of the inflatable body 2012 can be a hinge-like structure that unbends and inverts at a lower activation pressure, leading to a round cross section of the inflated device at a lower activation pressure, as shown in FIG. 11C. Then as activation pressure is increased, the second section 2014 stretches to expand the size of the inflatable body 2012, as shown in FIG. 12B. In other words, the first section 3013 of the inflatable body 2012 can be inflated prior to an inflation of the second section 2014 which is composed of an elastomeric membrane component. The pressure at which the first section 3013 unfolds may be, for example, between 1 and 20 psi, while the pressure at which the second section 2014 stretches may be, for example, in the range from 5 to 200 psi In an exemplary embodiment, the first section 3013 may completely unfold at 5 to 10 psi, leading to a total diameter of the inflatable body 2012 of 3 millimeters, for example, while expansion of the second element 2014 is minimal prior to addition of 10 psi, but increases sharply from 10 psi to 40 psi and leads to growth of the diameter from 3 to up to 20 mm.

As shown in FIG. 11C, in the first expanded configuration, the first section 3013 of the inflatable body 2012 has reached its rounded shape while the second section 2014 does not start to inflate or stretch. The tissue penetrating member 2004 can be sheathed and protected by the protective element during the inflatable body transitioning from the configuration shown in FIG. 10B to the transitional configuration shown in FIG. 11B and then the first expanded configuration shown in FIG. 11C. A pattern/shape of the protective elements 2015 in the first expanded configuration can change with respect to the pattern/shape of the protective elements in the involuted contracted configuration.

The inflatable body 2012 shown in FIGS. 12A and 12B has a second expanded configuration where the inflatable body is fully inflated by the increased hydraulic pressure in the inflatable body. The inflatable body 2012 in the second expanded configuration can have a larger profile than the first expanded configuration as both the first section 3013 and the second section 2014 of the inflatable body 2012 have reached their rounded shape. A coupling between the tissue penetrating member 2004 and the exterior surface of the first section 3013 of the inflatable body 2012 can be maintained due to a flexibility of the flexible drug line tubing 2005. In other words, the flexible drug line tubing 2005 can be deformed to conform to the expanded exterior surface of the first section 3013. The tissue penetrating member 2004 can remain in fluidic communication with the injection lumen of the catheter shaft assembly via the flexible drug line tubing 2005 in this second expanded configuration, such that a therapeutic or diagnostic agent can be delivered to the target region of the patient through the tissue penetrating member.

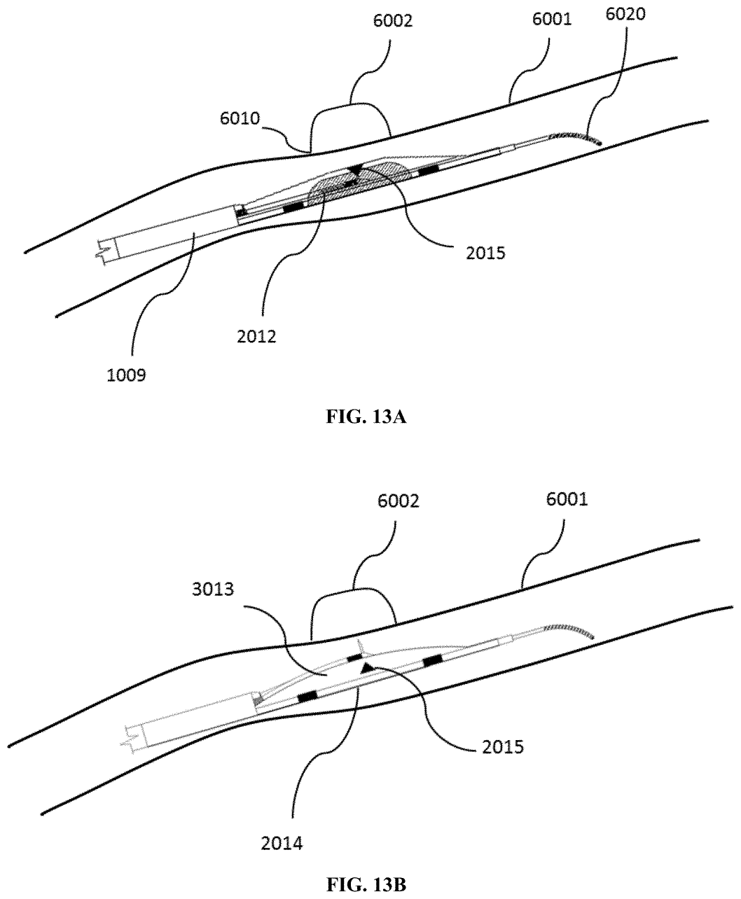

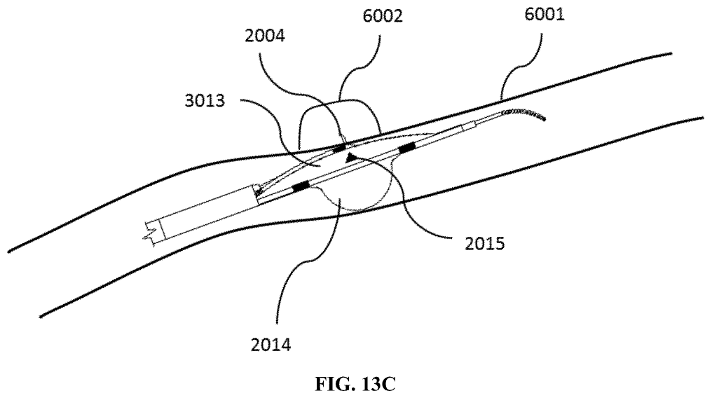

FIG. 13A shows the medical instrument for localized drug delivery as being inserted into a patient's body lumen. The catheter shaft assembly 1009 of the medical instrument can be inserted through an opening in the body (e.g., for bronchial or sinus treatment) or through a percutaneous puncture site (e.g., for artery or venous treatment) of the patient and moved within the patient's body lumen 6001, until a target region 6010 is reached. The catheter shaft assembly can be inserted and moved in the body lumen in the involuted contracted configuration where the inflatable body has a minimum profile and the tissue penetrating member (e.g., needle) is not deployed.

The target region 6010 can be a region where the body lumen tissue 6002 is positioned, and the body lumen tissue 6002 can be the tissue to which the therapeutic or diagnostic agents are to be delivered. The target region 6010 can be the site of tissue inflammation or more usually can be adjacent the sites typically being within 100 mm or less to allow migration of the therapeutic or diagnostic agent. The catheter shaft assembly can follow a guide wire 6020 that has previously been inserted into the patient. Optionally, the catheter shaft assembly can also follow the path of a previously-inserted guide catheter (not shown) that encompasses the guide wire.

As the catheter shaft assembly is guided inside the patient's body, the inflatable body 2012 can remain deflated and the needle can be held inside the U-shaped inflatable body, such that no trauma is caused to the body lumen walls. During maneuvering of the catheter shaft assembly, an imaging technique can be used to image the catheter shaft assembly and assist in positioning the inflatable body and the tissue penetrating member at the target region. The imaging technique can include at least one of a fluoroscopy, X-ray, or magnetic resonance imaging (MRI). For instance, the protective elements 2015 can be radio-opaque to provide feedback on X-ray imaging of the tissue penetrating member and/or the inflatable body. The protective elements 2015 can be provided with a specific pattern/shape such as an isosceles triangle shape with the vertex pointing downwards. For instance, the operator of the medical instrument can determine from this specific isosceles triangle shape with the vertex pointing downwards on the X-ray imaging that the inflatable body is not fully inflated (e.g., in the involuted contracted configuration or the first expanded configuration).

FIG. 13B shows the medical instrument for localized drug delivery as the inflatable component being partially inflated in the patient's body lumen. After being positioned at the target region, a movement of the catheter shaft assembly can be terminated and the hydraulic fluid can be supplied into the inflatable body, causing the inflatable body to inflate into the first expanded configuration where the first section 3013 of the inflatable body is inflated/expanded while the second section 2014 of the inflatable body maintains deflated. As shown, in the first expanded configuration, the first section 3013 of the inflatable body 2012 has reached its rounded shape while the second section 2014 does not start to meaningfully inflate or stretch. The inflated first section 3013 can touch the lumen wall which is opposite to the body lumen tissue 6002, and can raise/move the inflatable body towards the body lumen tissue 6002. The second section of the inflatable body 2014 may not be expanded in the first expanded configuration. This is particularly useful in smaller vessels where the second section 2014 of the inflatable body 2012 is not required to expand in order to penetrate the tissue penetrating element through the vessel wall. In larger vessels, additional pressure may cause the second section 2014 of the inflatable body 2012 to stretch and the inflatable body 2012 may reach a larger diameter to seat the penetrating element into and through vessel wall.

FIG. 13C shows the medical instrument for localized drug delivery as the inflatable body being fully inflated and the tissue penetrating member being deployed to penetrate into a luminal wall. The inflatable body can be converted into the second expanded configuration from the first expanded configuration as the hydraulic pressure in the inflatable body increases as a result of a continuous supplement of the hydraulic fluid from the inflation lumen. In the second expanded configuration, the inflatable body can be fully inflated where both the first section 3013 and the second section 2014 of the inflatable body reach their fully expanded shape. The inversion of the first section 3013 of the inflatable body can move the tissue penetrating member 2004 in a direction substantially perpendicular to the axis of the catheter shaft assembly to puncture the wall of the body lumen 6001 and advance into the body lumen tissue 6002 as well as the adventitia, media, or intima surrounding body lumens. For instance, the tissue penetrating member can be moved by the second section of the inflatable body beyond an external elastic lamina (EEL) of a blood vessel. The inflated second section 3013 of the inflatable body can allow contacting/abutting against the lumen wall which is opposite to the body lumen tissue 6002 during the tissue penetrating member puncturing into the body lumen tissue, such that a penetration depth of the tissue penetrating member can be maximized as a result of a supporting from the inflated first section.

As shown in FIG. 13C, a pattern/shape of the protective elements 2015 can be changed with respect to that shown in FIG. 13A and FIG. 13B. The operator of the medical instrument can determine from this change in the pattern/shape of the protective elements that an inflation status of the inflatable body and/or a development status of the tissue penetrating member have been changed. This change in the pattern/shape of the protective elements on X-ray imaging of the inflatable component can function as an indicator that the tissue penetrating member has been fully deployed.

After actuation of the tissue penetrating member (e.g., needle) and delivery of the drugs/agents to the target region through the tissue penetrating member, the hydraulic fluid can be exhausted from the inflatable body, causing the inflatable body to return to its original, involuted contracted state. The tissue penetrating member, being withdrawn, can once again be sheathed by the protective element. Once the inflatable body is deflated and the tissue penetrating member is withdrawn, the catheter shaft assembly can either be repositioned for further drug delivery or withdrawn from the patient's body lumen.

The hydraulic pressure useful to cause actuation of the inflatable body is typically in the range from 0.1 atmospheres to 20 atmospheres, more typically in the range from 0.5 to 20 atmospheres, and often in the range from 1 to 10 atmospheres. It may take only between approximately 100 milliseconds and five seconds for the tissue penetrating member to move from its furled state to its unfurled state.

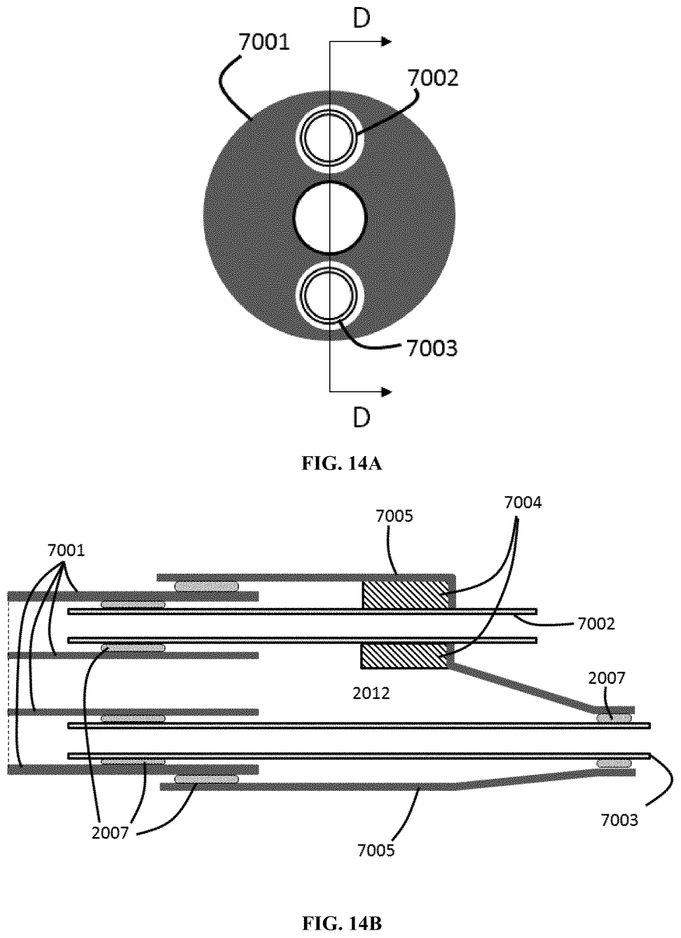

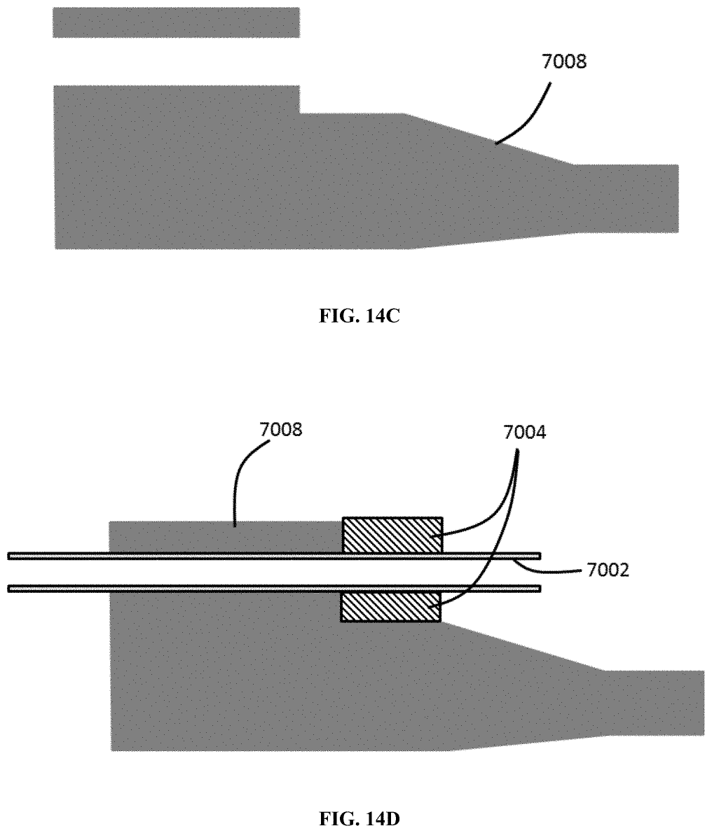

FIG. 14A shows an embodiment useful for routing fluids from a multi-lumen catheter tubing 7001 into separate lumens 7002 and 7003 and an expandable cavity 2012. FIG. 14B is a cross-sectional view along line D-D of FIG. 14A. The cavity may be bound by the walls of an expandable element defined by walls 7005, for example, like the balloon in FIG. 10B, where walls 7005 can form the structure defined by walls 3013 and 2014. In routing fluids from the multi-lumen catheter tubing 7001, manufacturing challenges arise in sealing the tubings if they are required to traverse through a pressurized element like cavity 2012. A variety of embodiments are provided in the present disclosure. In the first exemplary embodiment, as shown in FIG. 14A, an open lumen of the multi-lumen catheter tubing 7001 can be routed into tube 7002, which traverses the wall 7005 of cavity 2012. This can be implemented by first coating a portion of the outside of tube 7002 with an elastomeric adhesive (such as RTV silicone or other thermoplastic elastomer) and placing it in contact with a dissolvable mold in the shape of the walls 7005 of cavity 2012. The dissolvable mold 7008 is shown in FIG. 14C and FIG. 14D.

In FIG. 14C, the tubing 7002 has been added in and elastomeric adhesive 7004 has been coated around the outlet junction of tube 7002 and dissolvable mold 7008. Upon coating with a material to form walls 7005 in FIG. 14A (such material may be a vapor deposited polymer such as parylene or may be a dip-coated polymer such as polyimide or the like), the seal around 7002 can be fully formed. Upon removal of the dissolvable mold by common methods of polymer dissolution, the structure formed by walls 7005, tube 7002 and elastomeric material 7004 can be left. Returning to FIG. 14A, this structure may be bonded with adhesive 2007 into the multi-lumen catheter tubing at each tubing junction (7002 to 7001, 7003 to 7001, 7003 to 7005, and 7005 to 7001) to fully form the cavity 2012, which is fluidically isolated from the interior of tubing 7002 and 7003. In the exemplary example where parylene vapor deposition is applied onto RTV adhesive, a strong material bond can be obtained due to the chemical bonds formed during deposition. In this exemplary example, tubes 7002 and 7003 can be made of polyimide, pebax, PEEK, or other common medical plastics. Adhesive 2007 can be cyanoacrylate, light-cured adhesive, or other medical adhesive. Catheter tubing can be comprised of pebax, polyurethane, nylon, or other medical tubing material. Catheter 7001 can be approximately 0.5 to 4 mm in diameter, and tubings 7002 and 7003 can be approximately 0.1 to 2 mm in diameter.

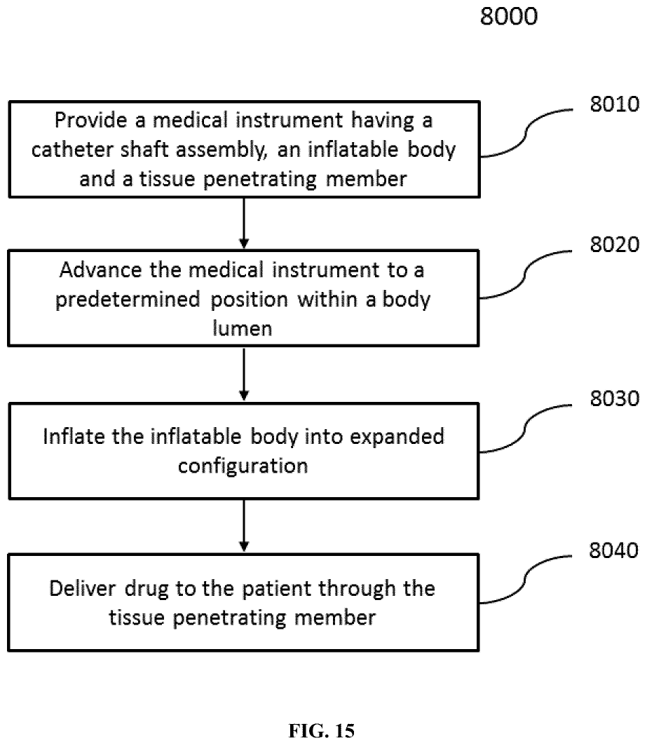

FIG. 15 shows a method 8000 for delivering a drug to a patient in accordance with some embodiments of the disclosure. The method can be performed to deliver a pharmaceutical drug or a diagnostic agent to a patient's body lumen using the medical instrument for localized drug delivery provided in this disclosure. In step 8010, a medical instrument as described with reference to FIGS. 8 to 14 of the disclosure can be provided. The medical instrument can comprise a catheter shaft assembly and a hub coupled to a proximal end of the catheter shaft assembly. The catheter shaft assembly can include a catheter body tubing with one or more lumens such as an injection lumen, in inflation lumen and a guidewire lumen. The medical instrument can comprise an inflatable component provided at a distal end of the catheter shaft assembly. The inflatable component can comprise an inflatable body and at least one protective element provided at the inflatable body. The inflatable body can be inflated from an original involuted contracted configuration to a first expanded configuration and then a second expanded configuration as a hydraulic pressure inside the inflatable body gradually increases. At least one tissue penetrating member (e.g., a needle) can be coupled to the inflatable body in an orientation transverse to the longitudinal axis of the catheter shaft assembly. The at least one protective element can be coupled to the inflatable body at a position in proximity to the tissue penetrating member. For instance, the protective element can be placed to surround to the sharp needle tip of the tissue penetrating member and function to protect the inflatable body from needle tip penetration or damage during transit of the medical instrument into and out of the body lumen.

In step 8020, the medical instrument can be advanced over a guidewire to a predetermined position within the body lumen of the patient when the inflatable component is in the involuted contracted configuration. The catheter shaft assembly of the medical instrument can be inserted through an opening in the body or through a percutaneous puncture site of the patient and moved within the patient's body lumen, until a target region is reached. The catheter shaft assembly can be inserted and moved in the body lumen in the involuted contracted configuration where the inflatable body has a minimum profile. During a delivery of the catheter shaft assembly, an imaging technique such as X-ray or magnetic resonance imaging (MRI) can be used to assist in positioning the inflatable body and the tissue penetrating member at the target region. For instance, the protective elements can be radio-opaque to provide feedback on X-ray imaging of the tissue penetrating member tip/inflatable body.

In step 8030, the inflatable component can be inflated into the second expanded configuration when the catheter shaft assembly is at the predetermined position in the body lumen. The hydraulic fluid can be supplied into the inflatable body when the catheter shaft assembly is positioned at the target region, causing the inflatable body to inflate into the first expanded configuration where only the first section of the inflatable body is inflated and then into the second expanded configuration where both the first section and the second section of the inflatable body are inflated to fill the body lumen. In the second expanded configuration, the inflatable body can be fully inflated and the tissue penetrating member can be moved in a direction substantially perpendicular to the axis of the catheter shaft assembly to puncture the wall of the body lumen and advance into the body lumen tissue. In some embodiments, the method for delivering a drug to a patient can further comprise observing an orientation change of the at least one protective element to confirm an inflation of the inflatable body as inflating the inflatable body changes the orientation of the at least one protective element.

In step 8040, the drug can be delivered to the patient through the tissue penetrating member which is in fluid communication with the injection lumen. The tissue penetrating member can be coupled to the injection lumen via the flexible drug line tubing. For instance, the distal end of the flexible drug line tubing proximal to the location that the tissue penetrating member bends upright can be affixed to an exterior surface of the inflatable body, and a shaft end of the tissue penetrating member can be coupled to the distal end of the flexible drug line. Due to a flexibility of the flexible drug line tubing, the distal end of the flexible drug line tubing can be fixed on the exterior surface of the inflatable body during an inflation of the inflatable body, thus the tissue penetrating member is maintained upright with respect to the exterior surface of the inflatable body during an inflation of the inflatable body. Once the drug delivery is completed, the hydraulic fluid can be exhausted from the inflatable body, causing the inflatable body to return to its original, involuted contracted state. The tissue penetrating member can then be either repositioned for further agent delivery or withdrawn from the patient's body lumen.

Although the above steps show method 8000 in accordance with many embodiments, a person of ordinary skill in the art will recognize many variations based on the teaching described herein. The steps may be completed in a different order. Steps may be added or deleted. Some of the steps may comprise sub-steps. Many of the steps may be repeated as often as beneficial.

The Bullfrog device is a catheter useful in delivering drugs to the perivascular tissue surrounding arteries and veins in the body that fall within the diameter range printed on the Bullfrog labeling. The Bullfrog has an articulating microneedle that is pressed through the blood vessel wall from the inside when the Bullfrog balloon is inflated. When deflated, the Bullfrog balloon shields the needle from scratching against the vessel wall during catheter manipulation and placement. In some instances, the balloon for use in veins may be larger than that for use in arteries as veins usually have lumens with a larger diameter and a larger circumference than lumens of arteries.

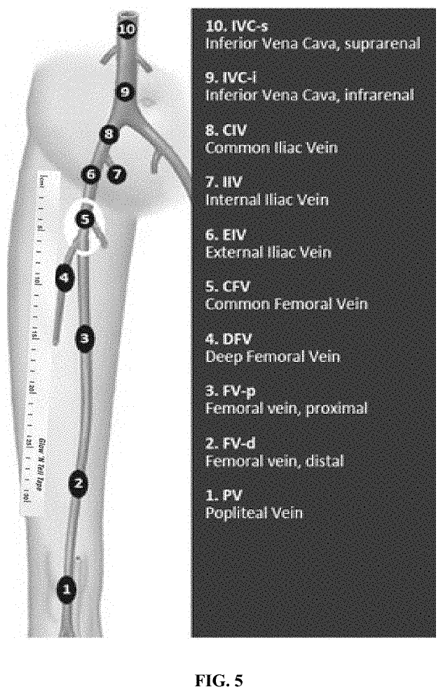

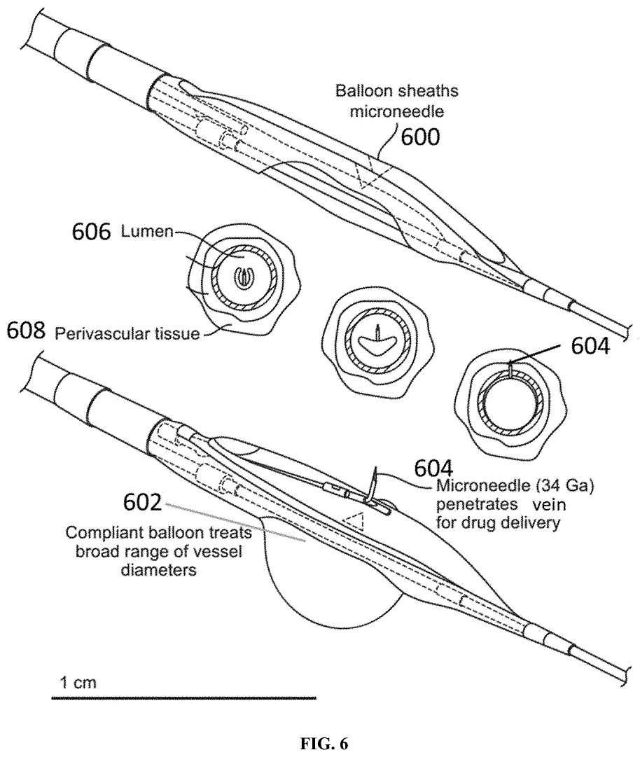

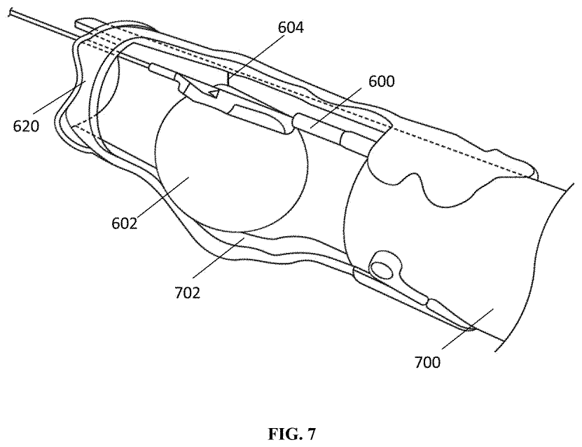

The Bullfrog Micro-Infusion Device is a CE-marked and FDA 510(k)-cleared device for the delivery of medications into the perivascular space of peripheral vessels. Dexamethasone is indicated for soft tissue injection to reduce inflammation. In some instances, thrombosis and subsequent vein fibrosis are known to be due to localized inflammation of the vein wall. In some instances, the delivery of dexamethasone into the perivenous tissue may decrease the early-stage inflammation that has been linked to reduction of patency. The pre-clinical and clinical studies using the Bullfrog Micro-Infusion Device to deliver commercially available dexamethasone at a dose of 1.6 mg per cm of artery have been safe and indicates no significant increase in the risks to patients in this study population. No dose-limiting toxicity has been observed. FIG. 6 shows an example of a needle injection catheter 600 having a balloon 602 that sheaths a microneedle 604. The compliant balloon 602 allows for treatment of a broad range of vessel diameters, including the larger vein diameters, and the microneedle penetrates the vein wall from the lumen 606 for drug delivery into the perivascular tissue 608. FIG. 7 show an example of a needle injection catheter 600 having an expandable balloon 602 and injection needle 604 delivering a therapeutic composition 620 into the perivascular space of a vein 700 affected by DVT at the thrombosed segment 702.

In some embodiments, the methods, devices, systems provided herein use a needle injection catheter. As shown in FIG. 5, a ruler may be placed on skin of target limb, running from the inguinal fold downward and on the medial side of the leg. Prior to endovenous intervention, a radio-opaque ruler may be placed on the skin as shown in FIG. 5, along the anterolateral or posterolateral surface of the thigh and with the 0-cm mark aligned at the inguinal fold. In some embodiments, the various points for endovenous intervention may include popliteal vein (PV) 1, distal femoral vein (FV-d) 2, proximal femoral vein (FV-p) 3, deep femoral vein (DFV) 4, common femoral vein (CFV) 5, external iliac vein (EIV) 6, internal iliac vein (IIV) 7, common iliac vein (CIV) 8, infrerenal inferior vena cava (IVC-i) 9, and suprarenal inferior vena cava (IVC-s) 10.

In some embodiments, the therapeutic delivering catheter may access the thrombosed vein segment from various entry sites. In some embodiments, the sheath of the therapeutic delivering catheter may enter the vasculature of the subject from one or more of popliteal vein, tibial vein, femoral vein, or iliac vein. In some embodiments, the therapeutic delivering catheter may access the thrombosed vein segment from behind the knee. In some embodiments, the therapeutic delivering catheter may access the thrombosed vein segment from popliteal vein. In some embodiments, the popliteal vein access is at the lower popliteal vein. In some embodiments, the therapeutic delivering catheter may access the thrombosed vein segment from tibial vein. In some embodiments, the therapeutic delivering catheter may access the thrombosed vein segment from femoral vein.

In some embodiments, the thrombosis may be treated before a therapeutic composition is delivered into the perivascular tissue surrounding a vein affected by the thrombosis. In some embodiments, a venogram and wire crossing of deep vein thrombosis may be performed. In some embodiments, a recanalization to remove non-adherent clot may be performed. In some embodiments, a venoplasty and stenting as needed to restore venous patency may be performed. In some embodiments, the therapeutic composition may be delivered to the perivascular tissue surrounding a vein affected by the thrombosis using the needle injection catheter.

In some embodiments, angiographic images may be captured of the venous segment affected by the thrombus before treatment and after treatment. In some embodiments, angiographic images may be captured of the venous segment affected by the thrombus to identify the vein and the thrombotic segment to be treated. In some embodiments, angiographic images may be captured of the venous segment affected by the thrombus after perivascular infusion, without luminal contrast infusion.

### Assessment and Endpoint Measurements

A number of endpoints may be measured to determine the effectiveness and safety of perivenous local delivery of therapeutic agents provided herein for treating symptoms of PTS and reducing progression to PTS. Various endpoints may be measured to determine the effectiveness and safety of perivenous local delivery of therapeutic agents provided herein for reducing inflammation and resolving thrombosis in affected veins. In some cases, the rate of clinically relevant loss of primary patency of the vein may be determined at months after thrombectomy in individuals with DVT. In some cases, the rate of clinically relevant loss of primary patency of the vein may be determined at 6 months after thrombectomy in iliofemoral or femoropopliteal DVT with extension below the inguinal ligament. In some cases, the endpoint measures are chosen to reduce investigator bias. In some cases, endpoints may be measured to determine the effectiveness and safety of perivenous local delivery of therapeutic agents and may include but are not limited to reduction of vascular inflammation as evidenced by levels of FDT-PET-detected metabolic activity surrounding the vein, levels of systemic circulating inflammatory biomarkers, extension of vascular patency as determined by duplex ultrasound at 6 months, or reduction in progression to post-thrombotic syndrome at 6 months and longer time points, out to 2 years.

In some cases, the rate of clinically relevant loss of primary patency may be measured to determine the effectiveness of the therapy. Reduction in the rate of re-thrombosis represents a clear clinical benefit to the patient that must be weighed against the risk of the catheter-based infusion of dexamethasone. Reducing the rate of clinically relevant (symptomatic) occlusions at 6 months would provide significant clinical benefit. Clinically relevant loss of primary patency may occur with (a) worsening or non-resolving symptoms of DVT and (b)(i) reintervention of the treated segment or (ii) ultrasound or angiographic detection of rethrombosis of the treated segment causing occlusion in unstented vein or ≥50% narrowing in stented vein. In some cases, measurement of occlusion may be performed with duplex ultrasound techniques.

In some cases, re-thrombosis may be an event that occurs in the midst of an inflamed vein that continues to recruit cells that lead to thrombus aggregation. In the event of re-thrombosis, multiple treatment/interventional possibilities exist, all of which have well established rates of complications. In some cases, the patient may first undergo an interventional procedure which carries various risks (e.g., bleeding complications, contrast complications). In some cases, thrombosis may have occurred, requiring intervention (catheter-directed pharmaceutical thrombolysis or mechanical thrombectomy) to clear the thrombus. In some cases, an alternative to catheter-directed therapy may be the medical management of the patient with oral anticoagulants, depending on the degree of re-thrombosis. In some cases, in the absence of thrombosis, where fibrotic tissue has built up and occluded the vein, venoplasty with or without stenting may be attempted. In some cases, initially or subsequently, the patient may experience long-term and chronic complications including pain, swelling, redness and ulceration of the affected leg. Avoidance of re-thrombosis at 6 weeks would represent a dramatic improvement over the current state-of-the-art therapeutic interventions and would provide a clear clinical benefit to the patient.

In some cases, the effectiveness of the treatment to maintain clinically relevant primary patency of the target vein segment may be assessed by measuring the rate of clinically relevant primary patency overall and in each segment (CIV, EIV, CFV, PFV, FV, POP). In some cases, clinically relevant loss of primary patency may be observed with (a) worsening or non-resolving symptoms of DVT and (b)(i) reintervention of the treated segment or (ii) ultrasound or angiographic detection of rethrombosis of the treated segment causing occlusion in unstented vein or ≥50% narrowing in stented vein. In some cases, the measurements may be taken at discharge and one or more subsequent time points. In some cases, primary patency may be defined by an unoccluded target vein segment without re-intervention.

In some cases, to assess the effectiveness of the treatment to maintain primary patency of the target vein segment, the rate of primary patency overall and in each segment (CIV, EIV, CFV, PFV, FV, POP) may be measured. In some cases, loss of primary patency is observed with ultrasound or venographic detection of complete occlusion of the treated fem-pop segment or a clinically driven re-intervention of the treated segment. In some cases, the measurements may be taken at discharge and one or more subsequent time points.

In some cases, to assess the effectiveness of the treatment to maintain primary assisted patency of the target vein segment, the rate of primary assisted patency may be measured. In some cases, the loss of primary assisted patency may occur with the first complete occlusion of the unstented or stented segment in the target vein, as detected by ultrasound or venography. In some cases, the measurements may be taken at discharge and one or more subsequent time points. In some cases, primary assisted patency may describe the cases where the vein remains functional even when an intervention has been required to keep it open.

In some cases, to assess the effectiveness of the treatment to maintain secondary patency of the target vein segment, rate of secondary patency is measured. In some cases, the loss of secondary patency occurs with permanent occlusion of the unstented or stented segment in the target vein, as detected by ultrasound or venography. In some cases, secondary patency describes the case where the vein can be returned to functional status even after it has been occluded after the initial intervention. This is also often referred to as cumulative patency. In some cases, the measurements may be taken at discharge and one or more subsequent time points.

In some cases, to assess the effectiveness of the treatment to limit the need for clinically driven target vein reintervention, the time to first clinically driven reintervention may be recorded. In some cases, reducing reintervention rate may be important to improving patient quality of life. In some cases, the need for reintervention may be tied to worse late-stage outcomes.

In some cases, to assess the effectiveness of the treatment to limit the rate of venous reflux, the rate of venous reflux (time cutoff 1000 ms) as measured by ultrasound may be taken. In some cases, venous reflux may indicate dysfunctional valves, which is a key characteristic of PTS.

In some cases, to assess limit in the progression to PTS, the PTS rate may be assessed by the PTS rate by Villalta score and VCSS score. Villalta and VCSS scoring systems are commonly used to determine progression and severity of PTS. In some cases, Villalta score of ≥5 or VCSS score ≥4 indicates progression to PTS. To assess the limit in the overall severity of PTS by the treatment, the rate of PTS by Villata and VCSS score may be taken at multiple time points after administration. A rate of mild PTS has a Villalta score of 5-9, moderate PTS by Villalta score of 10-14, of severe PTS by Villalta score of 15 or greater. A rate of mild-to-moderate PTS has a VCSS score of 4-7, of severe PTS by VCSS score ≥8. In some cases, to assess the maintenance of reduced Villalta and VCSS scores versus baseline, a change in Villalta score and VCSS score from baseline to follow-up at 3, 6, 12, 18, and 24 months may be taken. Evidence of improvement in Villalta or VCSS scores can be useful to demonstrate a clinically significant benefit of the treatment.

In some cases, the effectiveness of the treatment to limit the leg pain may be measured by a change from baseline to each follow-up using a Likert 7-point pain scale. In some cases, reducing leg pain may be a key component in improving a participant's quality of life.

In some cases, to assess the effectiveness of the treatment to limit the index-leg minimal circumference as measured by a change from baseline, minimal target leg circumference as measured at 10 cm below the tibial tuberosity of the target leg may be taken after administration. The index leg circumference helps to determine the degree of edema that a patient is experiencing.

In some cases, to assess the effectiveness of the treatment for improvement in quality-of-life outcomes, VEINES questionnaire (25-question VEINES-QOL and 10-question VEINES-Sym) may be taken at baseline and at one or more follow-up. The VEINES questionnaires are commonly accepted within the field of DVT to establish participant quality of life.

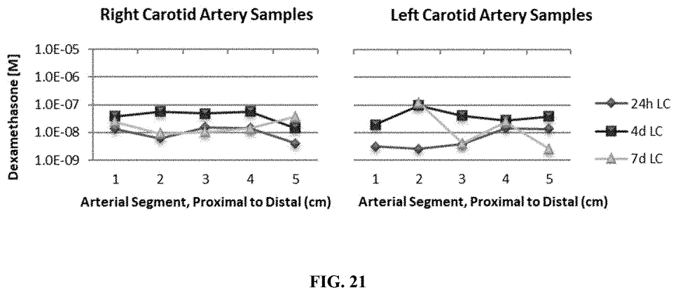

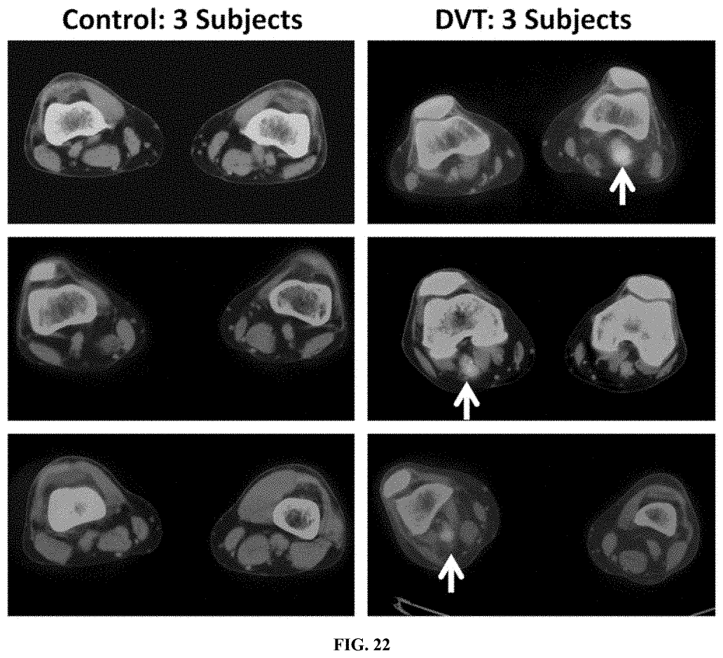

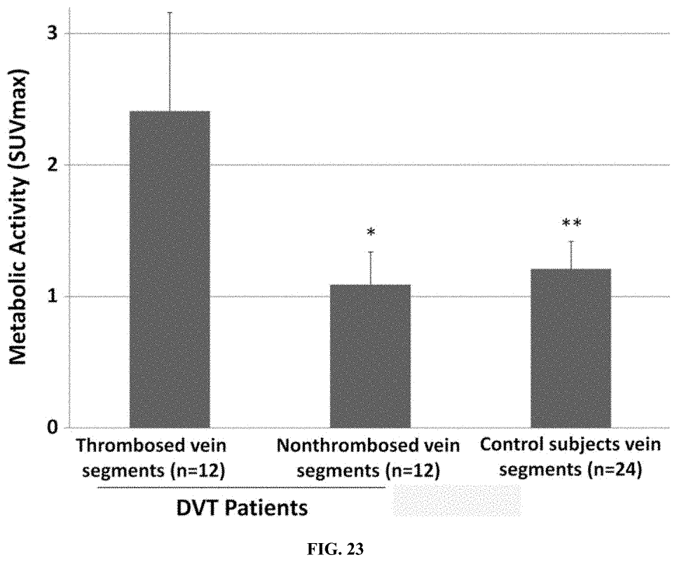

In some cases, to assess the effectiveness of the treatment, the level of metabolic activity surrounding the target vein may be measured by FDG-PET. FIG. 20 illustrates the results of FDG-PET in three DVT subjects, in which inflammation may be imaged based on increased metabolic activity surrounding the inflamed vein, wherein the increase in metabolic activity is detectable via FDG-PET signal. In FIG. 21, this signal strength is displayed as the Metabolic Activity (SUVmax), where thrombosed segments have more than twice the metabolic activity as non-thrombosed vein segments in DVT patients or in control segments in non-thrombosed patients. In some cases, the delivery of anti-inflammatory medication around the target vein may reduce metabolic activity from levels that may be 2 to 4 times normal levels, such as exist in a contralateral, non-diseased segment. In some cases, the metabolic activity levels may be reduced by up to 25%, up to 50%, or back to approximately normal in comparison to non-diseased segments.

In some cases, to assess the effectiveness of the treatment, the levels of circulating inflammatory biomarkers and change from baseline may be measured in order to determine systemically detectable changes in inflammation. In some cases, the levels of circulating inflammatory biomarkers and their change from baseline to follow-ups may be assessed for one or more of the following biomarkers: IL-1β, IL-2, IL-6, IL-8, IL-10, IFN-α, IFN-γ, ICAM-1, TNF-α, hsCRP, D-dimer, and fibrinogen. In some cases, measuring the circulating biomarkers may provide important data to determine which inflammatory molecules are being reduced vs. those that are not. Inflammatory biomarker levels may be linked to progression to PTS.

In some cases, to assess the safety of the treatment, the ability of the treatment to limit the rate of serious adverse events in the first 30 days after treatment is assessed. Also, the ability of the treatment to limit adverse events (subclassified as major, serious, non-serous, unanticipated, revascularization-procedure-related, device-related and drug-related) may be assessed.

In some cases, to assess the technical success of the treatment, complete longitudinal and circumferential distribution of drug around the target vein segment may be assessed by infusion grade and coverage % by angiography during the procedure. In some cases, the distribution pattern achieved during the adventitial and perivascular drug delivery can be used to potentially correlate drug distribution pattern to positive outcomes.

The VCSS score may be ascertained at each listed visit. The score is determined by adding the scores from the list of 10 categories below in Table 5, with a total score ranging from 0 to 30.

TABLE 5
VCSS Score criteria

| Score: | None: 0 | Mild: 1 | Moderate: 2 | Severe: 3 |
|---|---|---|---|---|
| Pain or other | | Occasional pain or | Daily pain or | Daily pain or discomfort |
| discomfort (i.e., aching, | | other discomfort (i.e., | other discomfort | (i.e., limits most regular |
| heaviness, fatigue, | | not restricting regular | (i.e., interfering | daily activities) |
| soreness, burning) | | daily activities) | with but not | |
| Presumes venous | | | preventing | |
| origin. | | | regular daily | |
| | | | activities) | |
| Varicose veins | | Few: scattered (i.e., | Confined to calf | Involves calf and thigh |
| “Varicose” veins must | | isolated branch | or thigh | |
| be >3 mm in diameter | | varicosities or | | |
| to qualify in the | | clusters) Also includes | | |
| standing position. | | corona phlebectatica | | |
| | | (ankle flare) | | |
| Venous edema | | Limited to foot and | Extends above | Extends to knee and above |
| Presumes venous | | ankle area | ankle but below | |
| origin. | | | knee | |
| Skin pigmentation | None | Limited to | Diffuse over | Wider distribution above |
| Presumes venous | or | perimalleolar area | lower third of | lower third of calf |
| origin. | focal | | calf | |
| Does not include focal | | | | |
| pigmentation over | | | | |
| varicose veins or | | | | |
| pigmentation due to | | | | |
| other chronic diseases. | | | | |
| Inflammation | | Limited to | Diffuse over | Wider distribution above |
| More than just recent | | perimalleolar area | lower third of | lower third of calf |
| pigmentation (i.e., | | | calf | |
| erythema, cellulitis, | | | | |
| venous eczema, | | | | |
| dermatitis) | | | | |
| Induration | | Limited to | Diffuse over | Wider distribution above |
| Presumes venous origin | | perimalleolar area | lower third of | lower third of calf |
| of secondary skin and | | | calf | |
| subcutaneous changes | | | | |
| (i.e., chronic edema | | | | |
| with fibrosis, | | | | |
| hypodermitis). Includes | | | | |
| white atrophy and | | | | |
| lipodermatosclerosis. | | | | |
| Active ulcer number | 0 | 1 | 2 | ≥3 |
| Active ulcer duration | N/A | <3 mo | >3 mo but <1 y | Not healed for >1 y |
| (longest active) | | | | |
| Active ulcer size | N/A | Diameter <2 cm | Diameter 2-6 | Diameter >6 cm |
| (largest active) | | | cm | |
| Use of compression | Not | Intermittent use of | Wears stockings | Full compliance: |
| therapy | used | stockings | most days | stockings |

In some cases, a target leg exam may be used to examine the target leg for clinical signs of venous disease and characterize by CEAP classification schema. The schema includes: Clinical: C0—No clinical signs, C1—Small varicose veins, C2—Large varicose veins, C3—Edema, C4—Skin changes without ulceration, C5—Skin changes with healed ulceration, C6—Skin changes with active ulceration; Etiology: EC—Congenital, EP—Primary, ES—Secondary (usually due to prior DVT); Anatomy: AS—Superficial veins, AD—Deep veins, AP—Perforating veins; Pathophysiology: PR—Reflux, PO—Obstruction. In some cases, as part of the target leg exam, three circumferences at ankle, calf, and thigh may be measured.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining”, “measuring”, “evaluating”, “assessing,” “assaying,” and “analyzing” are often used interchangeably herein to refer to forms of measurement and include determining if an element is present or not (for example, detection). These terms can include quantitative, qualitative or quantitative and qualitative determinations. Assessing is alternatively relative or absolute. “Detecting the presence of” includes determining the amount of something present, as well as determining whether it is present or absent.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. The disease can be endometriosis. In some cases, the subject is not necessarily diagnosed or suspected of being at high risk for the disease.

The term “in vivo” is used to describe an event that takes place in a subject's body.

The term “ex vivo” is used to describe an event that takes place outside of a subject's body. An “ex vivo” assay is not performed on a subject. Rather, it is performed upon a sample separate from a subject. An example of an “ex vivo” assay performed on a sample is an “in vitro” assay.

The term “in vitro” is used to describe an event that takes places contained in a container for holding laboratory reagent such that it is separated from the living biological source organism from which the material is obtained. In vitro assays can encompass cell-based assays in which cells alive or dead are employed. In vitro assays can also encompass a cell-free assay in which no intact cells are employed.

As used herein, the term “about” a number refers to that number plus or minus 10% of that number. The term ‘about’ a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The section headings used herein are for organizational purposes only and are not to be construed as limiting the subject matter described.

### EXAMPLES

The following examples are included for illustrative purposes only and are not intended to limit the scope of the invention.

### Example 1

### In Vivo Mouse Study

Provided herein is a pre-clinical study to examine the feasibility of therapeutic dexamethasone local delivery to the perivascular surrounding tissue of venous thrombus in a mouse model of deep vein thrombosis (DVT) induced by inferior vena cava (IVC) ligation. A total of 30 mice were evaluated in this study. On day 0, deep vein thrombus (DVT) was induced in the infrarenal IVC. On Day 2, the mice were injected with the control (10 subjects received injections of PBS with 1% methylene blue solution (final concentration of 0.2% methylene blue)) or low dose dexamethasone (10 subjects received injections of 4 mg/mL dexamethasone sodium phosphate with 1% methylene blue solution (final concentration of 3.2 mg/mL dexamethasone, final concentration of 0.5% methylene blue)) or high dose dexamethasone (10 subjects received injections of 10 mg/mL dexamethasone sodium phosphate with 2.5% methylene blue solution (final concentration of 8 mg/mL dexamethasone, final concentration of 0.5% methylene blue)) into the perivascular tissue surrounding IVC. The mice were sacrificed on Day 8, and RNA analysis (by PCR array analysis (i.e., inflammatory/fibrosis markers) with RNA extracted from the samples) or histology analysis was performed (n=5 per group). The histology analysis included measurements of vessel area, lumen area, vein wall area=Vessel area−Lumen area, % vein wall area=Vein wall area/Vessel area, vein thickness, thrombus area, organizing thrombus area, and % organizing area=Organizing area/Thrombus area. Inflammation of the IVC wall and within the outer one-fourth layer of the thrombus was assessed with semi-quantitative evaluation.

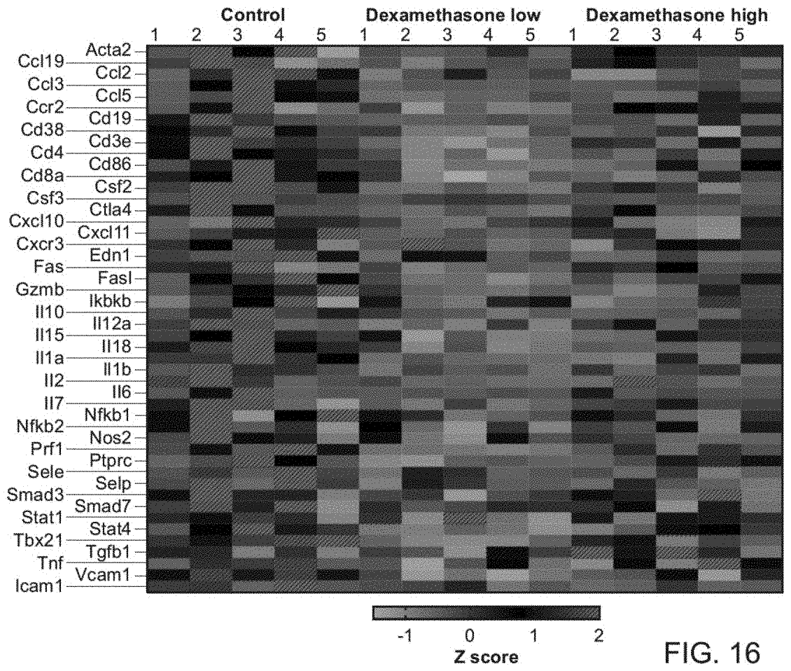

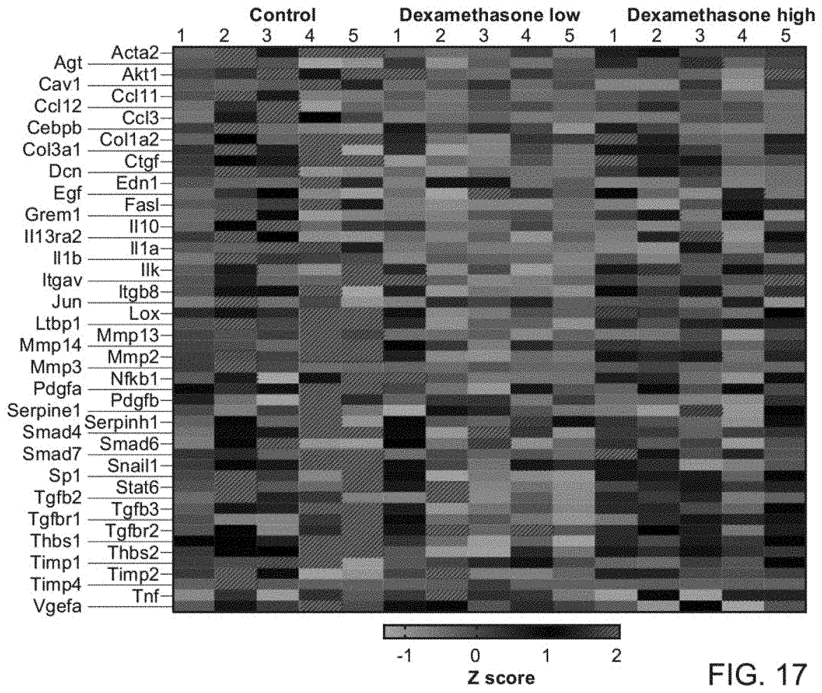

Thrombus weight was similar among the three groups (p=0.42). RNA was extracted from the DVT, inflammatory and fibrosis-related gene panels were assessed. RNA analysis revealed that the inflammatory genes, such as Cc12, Cxcl11, Cxcr3, IL-lb, IL-2, IL-6, IL-18, Nfkb1, Nfkb2, were significantly suppressed in both dexamethasone low- and high-dose groups compared with the control group as shown in FIG. 16. RNA analysis heat map of a portion of the inflammation panel shows the result of microarray RT-PCR for genes involved in TaqMan mouse immune response array plate as shown in FIG. 16. Inflammation-related genes were highly expressed in the control group while the inflammation-gene expression in both dexamethasone low dose and high dose groups were suppressed. This is in agreement with previous reports that glucocorticoids regulate these pro-inflammatory genes. Moreover, there was a trend in suppressing several fibrosis-related genes (Acta2, Colla2, Col3a1, MMP2, MMP13, MMP14, Tgfb2, Tgfb3, Timp1) in the dexamethasone group as shown in FIG. 17. However, the expression of the other fibrosis-related genes (e.g., Itgb, Smad6, Timp2, Thbs1, Thbs2, Vegfa) were similar among the three groups. The dexamethasone low dose group showed the same degree of reduced inflammatory gene expression as the high dose group.

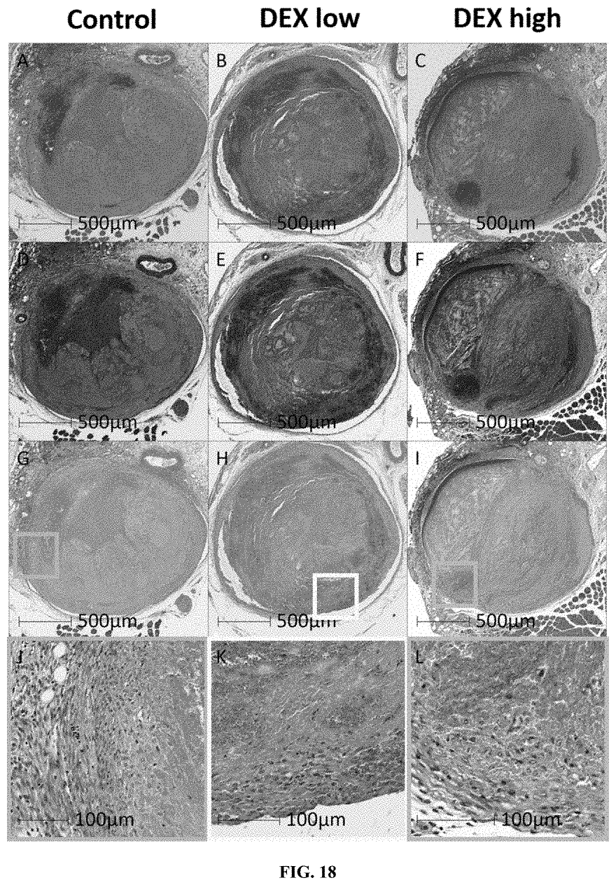

Histology evaluation revealed that the thrombus area, the IVC vein wall thickness, and vein wall area were similar among the three groups as shown in FIG. 18. Although the animal number was limited, the area of the organizing thrombus in the dexamethasone-treated group was smaller than the control group. However, there was no significant difference between the low dose and high dose dexamethasone groups. The dexamethasone-treated groups demonstrated less inflammation in the thrombus than the control group by semi-quantification. FIG. 18 shows representative histology images of the IVC and DVT, where panels A, D, G, J: Histology images of the control group. Low power field (A, D, G) from the control group with high-power image of boxed are in G shown in J. The edge of the thrombus shows advanced organization and adheres to the IVC wall. Inflammatory cell infiltration in the vessel wall and thrombus is observed. B, E, H, K: Histology images of the dexamethasone low dose group Low-(B, E, H) and high-power (K) fields from the dexamethasone low dose group. C, F, I, L: Histology images of the dexamethasone high dose group. Inflammatory cell infiltration within the thrombus was relatively less compared with the control case in the dexamethasone-treated groups. Less thrombus organization was observed in the dexamethasone-treated case. (A-C: Hematoxylin and eosin stain, D-F: Movat Pentachrome stain, G-I: Martius Scarlet Blue stain.)

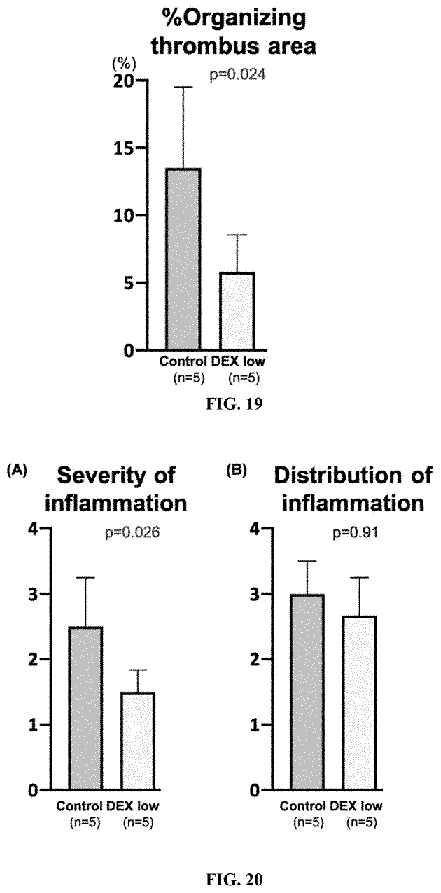

Percentage of the thrombus area occupied by organizing thrombus in an in vivo mouse study were similar amongst the groups. FIG. 19 shows that the area of organizing thrombus in the dexamethasone-treated group was significantly smaller than in the control group (p=0.024). FIG. 20 shows semi-quantitative evaluation of inflammation in the entire thrombus. in an in vivo mouse study. More severe inflammation was observed in the control group compared to the dexamethasone-treated groups. There were no significant differences in terms of the vein wall thickness or distribution of inflammation in the thrombus.

### Example 2

### In Vivo Pig Study

Provided herein is an in vivo pig study to examine the pharmacokinetics of perivenous local delivery of dexamethasone. Dexamethasone uptake and persistence in tissues has been demonstrated in a study of Bullfrog Micro-Infusion Device delivery of dexamethasone to the adventitial tissue of porcine carotid arteries. In this study, sustained levels in the range of 10 to 100 nM were seen 1, 4, and 7 days after infusion of 1 mg.

FIG. 21 shows dexamethasone levels measured in pig carotid arteries 1, 4, and 7 days after confirmed delivery of 1 mg dexamethasone sodium phosphate in 3 ml volume to the carotid artery adventitia with the Bullfrog Micro-infusion Device from an in vivo pig study. The delivery was made in segment 3 in each case. each line represents a single artery.

### Example 3

### In Vivo Pig Study

Provided herein is an in vivo pig study to examine the toxicity of perivenous local delivery of dexamethasone.

A first study was designed to compare a high dose of dexamethasone (10 mg equivalent dose of dexamethasone phosphate) delivered in 3 ml volume to the perivascular tissue of porcine AV grafts (6 mm ringed PTFE) implanted between femoral artery and femoral vein pairs, bilaterally. Fourteen days after graft implantation, percutaneous transluminal angioplasty (PTA) was performed (7 mm balloon, 16 atmosphere inflation pressure) at two sites per graft: across the graft-vein anastomosis (GVA) and in the proximal vein (PV). Perivascular infusion of either dexamethasone (6 grafts) or placebo (2 grafts) was administered following the PTA procedure. Infusions of 3m1 were always consistent between the 2 grafts in each animal. Animals were euthanized 14 days after the treatment procedure and the graft-vein anastomosis and proximal vein were analyzed by histopathology and histomorphometry to determine adverse effects from the high dose of dexamethasone. The study was not powered to identify differences in stenosis but rather was aimed at determining dexamethasone local toxicity.

The histopathology findings of the study indicated that femoral GVA treated with angioplasty and perivascular, high-dose dexamethasone, via Bullfrog Micro-Infusion Catheter, exhibited no differences compared to GVA treated with angioplasty and perivascular placebo.

A second study was designed to assess the local toxicity of dexamethasone administered via the Bullfrog Micro-Infusion Device in a swine model. The following objectives were met in the study: In each of four subjects, confirmation of the safety of up to 16 mg dexamethasone delivered to the adventitia and perivascular tissue of each of 4 peripheral arteries and 6.4 mg dexamethasone delivered to the adventitia and perivascular tissue of each of 3 coronary arteries (at 3.2 mg/mL with 20% contrast), as compared to normal untreated tissue in two untreated subjects by clinical pathology and histopathology examination, at 28±3 days. Measurement of dexamethasone plasma concentration at baseline, after each infusion and at 5 minutes, 1 hour, 24 hours, 7 days and 28±3 days after final dose administration to confirm removal of dexamethasone from systemic circulation. Measurement of dexamethasone tissue concentration at 28±3 days after dosing of 6.4 mg into each of three coronary arteries and 16 mg into each of four peripheral arteries within each of four subjects (individual doses per treated segment for a total of up to 83.2 mg dexamethasone per subject).

All animals successfully received the test article (dexamethasone delivered by Bullfrog device) without complication. None of the animals experienced adverse postoperative events leading to early death. Gross necropsy showed no evidence of injury to the treatment areas. In addition, peripheral organs did not reveal any abnormalities that could be associated with the administration of the test article.

Microscopic evaluation of tissues from six swine administered dexamethasone treatment with the Bullfrog Micro-Infusion Device and euthanized at 28±3 days or left as an untreated control and euthanized at 0 days showed the following: there was no evidence of local toxicity to the treated vessels and no evidence of local vascular irritation upon dexamethasone injection with the Mercator MedSystems Micro-Infusion Device. The injection procedure rarely caused minimal mural injury that was of no consequence on vascular healing or patency; namely there was no evidence of thrombosis or stenosis. The treated vessels were fully healed, generally showing a normal wall and occasionally displaying minimal to mild perivascular or adventitial fibrosis and low severity non-specific and localized mural inflammation considered to be of no pathological significance. There were isolated instances of media dissection in treated coronary arteries and a single instance of increased mural injury. These events were deemed to be procedural in origin and bore no relationships to dexamethasone injection.

The study concluded that based on evaluation of tissues from six swine administered dexamethasone treatment with the Mercator MedSystems Micro-Infusion Device or left as an untreated control, no adverse or toxicologically meaningful changes were present in the treated vessels. There was minor to occasionally mild procedural injury that was fully healed at the end of the study and produced no adverse consequences on the patency or healing of treated vessels.

### Example 4

### Perivenous Dexamethasone Therapy: Examining Reduction of Inflammation After Thrombus Removal to Yield Benefit in Subacute and Chronic Iliofemoral DVT (DEXTERITY-SCI)

Provided herein is a study to examine the effect of perivenous local delivery of dexamethasone on inflammation levels after thrombus removal in subacute and chronic inflammation in individuals with iliofemoral DVT. In some cases, the individuals also have symptoms of PTS.

This study is an interventional, multi-site, two-phase trial to examine the effect of Bullfrog® Micro-Infusion Device perivenous injection of dexamethasone sodium phosphate injection, USP, in a concentration of 3.2 mg/mL and dosage of 1.28 mg/cm to improve 6-month vessel patency after thrombectomy and stenting in symptomatic iliofemoral DVT with infrainguinal extension and late presentation (14-60 days post symptom onset). In the first phase (Lead-in Phase) of the trial, 20 participants are enrolled, and all are treated with dexamethasone. With confirmation of safety based on 6-week data from the first phase, the second phase (RCT Phase) of the trial has 40 participants in a 1:1 randomization receiving either dexamethasone (treatment) or sham saline (control) injections.

Description of Study Intervention: After completion of DVT recanalization (including baseline recanalization of de novo DVT and any re-intervention of the target vein through one year), participants qualify for enrollment in the study and receive treatment with the investigational drug (a solution containing 80% of 4.0 mg/mL dexamethasone sodium phosphate injection, USP, and 20% of contrast medium with >300 mg unbound iodine per mL) or sham (80% saline and 20% contrast medium with >300 mg unbound iodine per mL). Investigational drug or sham is delivered by Bullfrog Micro-Infusion Device to the adventitia and perivascular tissue around target vein segments. The dosage is delivered in a volume of 0.4 mL (1.28 mg) per cm of target vein length, up to 50 cm, for a total volume of up to 20 mL and a total dose of up to 64 mg dexamethasone.

Patients assigned to dexamethasone treatment in either the Lead-in Phase or the RCT Phase receive dexamethasone perivascular therapy at baseline intervention and then again at each DVT reintervention of their target vein for a one-year period. Similarly, patients assigned to control during the RCT Phase receive sham saline injections baseline and then again at each DVT reintervention of their target vein for a one-year period.

Study Hypothesis: In this study, the hypothesis is that negative outcomes including post-thrombotic syndrome (PTS) arise from post-thrombotic vein wall inflammation culminating in vein wall scarring, rethrombosis, loss of valve function, loss of venous patency and venous fibrosis due to inflammation. In some cases, the perivascular delivery of dexamethasone is intended to reduce deep vein thrombosis-related inflammation concomitant with removal of thrombus burden, relieving symptoms, reducing the potential for re-thrombosis and vein wall fibrosis, and thereby limiting loss of patency and resultant progression to re-thrombosis and/or occurrence or worsening of post-thrombotic syndrome.

Objectives and Endpoints: A number of endpoints are measured to determine the effectiveness and safety of perivenous local delivery of dexamethasone for treating subchronic and chronic inflammation due to DVT and PTS.

Rate of clinically relevant loss of primary patency is measured to determine the effectiveness of the therapy. Reducing the rate of clinically relevant (symptomatic) occlusions at 6 months would provide significant clinical benefit. Clinically relevant loss of primary patency occurs with (a) worsening or non-resolving symptoms of DVT and (b)(i) reintervention of the treated segment or (ii) ultrasound or angiographic detection of rethrombosis of the treated segment causing occlusion in unstented vein or ≥50% narrowing in stented vein. Timeframe for assessment is at about 6 months following the procedure. Measurement of occlusion may be performed with duplex ultrasound techniques.

To determine the safety of the therapy and to limit the incidence of composite major adverse events (MAE) at 30 days following treatment of an obstruction in the femoropopliteal segment, various measurements at 30 days following treatment including death, clinically significant pulmonary embolism (i.e., symptomatic, confirmed by CT pulmonary angiography), major (BARC 3b or greater) bleeding, target vessel thrombosis confirmed by imaging as assessed by core lab, infection of the treatment or insertion site, and AV fistula at the treatment site are measured.

To assess the effectiveness of the treatment to maintain clinically relevant primary patency of the target vein segment, the rate of clinically relevant primary patency overall and in each segment (CIV, EIV, CFV, PFV, FV, POP) are taken. In some cases, clinically relevant loss of primary patency may be observed with (a) worsening or non-resolving symptoms of DVT and (b)(i) reintervention of the treated segment or (ii) ultrasound or angiographic detection of rethrombosis of the treated segment causing occlusion in unstented vein or ≥50% narrowing in stented vein. The measurements are taken at discharge, 5 weeks, 3, 6, 12, 18 and 24 months. Primary patency may be defined by an unoccluded target vein segment without re-intervention. Recording those occlusions that are symptomatic improves understanding of clinical significance.

To assess the effectiveness of the treatment to maintain primary patency of the target vein segment, the rate of primary patency overall and in each segment (CIV, EIV, CFV, PFV, FV, POP) may be measured. In some cases, loss of primary patency is observed with ultrasound or venographic detection of complete occlusion of the treated fem-pop segment or a clinically driven re-intervention of the treated segment. The measurements are taken at discharge, 5 weeks, 3, 6, 12, 18 and 24 months.

To assess the effectiveness of the treatment to maintain primary assisted patency of the target vein segment, the rate of primary assisted patency was measured. The loss of primary assisted patency may occur with the first complete occlusion of the unstented or stented segment in the target vein, as detected by ultrasound or venography. The measurements are taken at 3, 6, 12, 18 and 24 months. Primary assisted patency may describe the cases where the vein remains functional even when an intervention has been required to keep it open.

To assess the effectiveness of the treatment to maintain secondary patency of the target vein segment, rate of secondary patency is measured. The loss of secondary patency occurs with permanent occlusion of the unstented or stented segment in the target vein, as detected by ultrasound or venography. The measurements are taken at 3, 6, 12, 18 and 24 months. Secondary patency describes the case where the vein can be returned to functional status even after it has been occluded after the initial intervention. This is also often referred to as cumulative patency.

To assess the effectiveness of the treatment to limit the need for clinically driven target vein reintervention, the time to first clinically driven reintervention was recorded at 5 weeks, 3, 6, 12, 18 and 24 months or unscheduled. The number of clinically driven reinterventions in the first year post enrollment is taken, and clinically driven reintervention rate (number of clinically driven reinterventions per year) over 24 months is taken. In some cases, reducing reintervention rate may be important to improving patient quality of life. In some cases, the need for reintervention may be tied to worse late-stage outcomes.

To assess the effectiveness of the treatment to limit the rate of venous reflux, the rate of venous reflux (time cutoff 1000 ms) as measured by ultrasound is taken at 6 and 12 months. In some cases, venous reflux may indicate dysfunctional valves, which is a key characteristic of PTS.

To assess limit in the progression to PTS, the PTS rate is assessed by the PTS rate by Villalta score ≥5 and by VCSS score ≥4 at 3, 6, 12, 18 and 24 months. Villalta and VCSS scoring systems are commonly used to determine progression and severity of PTS.

To assess the limit in the overall severity of PTS by the treatment, the rate of PTS by Villata and VCSS score are taken at 3, 6, 12, 18 and 24 months. A rate of mild PTS has a Villalta score of 5-9, moderate PTS by Villalta score of 10-14, of severe PTS by Villalta score of 15 or greater. A rate of mild-to-moderate PTS has a VCSS score of 4-7, of severe PTS by VCSS score ≥8.

To assess the maintenance of reduced Villalta and VCSS scores versus baseline, a change in Villalta score and VCSS score from baseline to follow-up at 3, 6, 12, 18 and 24 months are taken. Evidence of improvement in Villalta or VCSS scores can be useful to demonstrate a clinically significant benefit of the treatment.

To assess the effectiveness of the treatment to limit the leg pain (Likert 7-point scale) as measured by a change from baseline to each follow-up, the patients are assessed by the Likert pain scale at 5 weeks, 3, 6, 12, 18, and 24 months. In some cases, reducing leg pain may be a key component in improving a participant's quality of life.

To assess the effectiveness of the treatment to limit the index-leg minimal circumference as measured by a change from baseline, minimal target leg circumference as measured at 10 cm below the tibial tuberosity of the target leg are taken at 10 day and 5 weeks. The index leg circumference helps to determine the degree of edema that a patient is experiencing.

To assess the effectiveness of the treatment for improvement in quality-of-life outcomes, VEINES questionnaire (25-question VEINES-QOL and 10-question VEINES-Sym) were taken at baseline and each follow-up. Score from VEINES-QOL and VEINES-Sym, comparing follow up to baseline are taken at 10 day, 5 week, 3, 6, 12, 18 and 24 months. The VEINES questionnaires are commonly accepted within the field of DVT to establish participant quality of life.

To assess the effectiveness of the treatment, the levels of circulating inflammatory biomarkers and change from baseline are measured in order to determine systemically detectable changes in inflammation. The levels of circulating inflammatory biomarkers and their change from baseline to follow-ups at 10 days, 5 weeks, 3 months are assessed for one or more of the following biomarkers: IL-1β, IL-2, IL-6, IL-8, IL-10, IFN-α, IFN-γ, ICAM-1, TNF-α, hsCRP, D-dimer, and fibrinogen. In some cases, measuring the circulating biomarkers will provide important data to determine which inflammatory molecules are being reduced vs. those that are not. Inflammatory biomarker levels may be linked to progression to PTS.

To assess the safety of the treatment, the ability of the treatment to limit the rate of serious adverse events in the first 30 days after treatment is assessed. Also, the ability of the treatment to limit adverse events (subclassified as major, serious, non-serous, unanticipated, revascularization-procedure-related, device-related and drug-related) were assessed up to 24 months at 5 weeks, 3, 6, 12, 18 and 24 months.

To assess the technical success of the treatment, complete longitudinal and circumferential distribution of drug around the target vein segment were assessed by infusion grade and coverage % by angiography during the procedure. In some cases, the distribution pattern achieved during the adventitial and perivascular drug delivery can be used to potentially correlate drug distribution pattern to positive outcomes.

### Example 5

### Perivenous Dexamethasone Therapy: Examining Reduction of Inflammation After Thrombus Removal to Yield Benefit in Acute Femoropopliteal DVT (DEXTERITY-AFP)

Provided herein is a study to examine the effect of perivenous local delivery of dexamethasone on inflammation levels after thrombus removal in acute inflammation in individuals with femorpopliteal DVT. In some cases, the individuals also have symptoms of PTS.

This is an interventional, multi-site, two-phase trial to examine the effect of Bullfrog® Micro-Infusion Device perivenous injection of dexamethasone sodium phosphate injection, USP, in a concentration of 3.2 mg/mL and dosage of 1.28 mg/cm to improve patency 6 months after venous thrombectomy in symptomatic femoropopliteal deep vein thrombosis with or without proximal extension into the iliofemoral segment. In the first phase (the Lead-in Phase) of the trial, 20 participants will be enrolled, and all will be treated with dexamethasone. Upon confirmation of safety based on 6-week data from the Lead-in Phase, the second phase (the RCT Phase) of the trial will enroll 60 participants in a 1:1 randomization receiving either dexamethasone (treatment) or sham saline (control) injections.

The aim of this study is to determine the rate of clinically relevant loss of primary patency at 6 months after thrombectomy in femoropopliteal DVT with or without proximal extension into the iliofemoral segment. Typically, the patency loss in subjects experiencing similar symptoms and having gold-standard thrombolytic therapy may be approximately 60% at 6 weeks and 50% at 6 months. In some cases, mechanical thrombectomy may improve patency by 15-20%, but still leaves more than 35% of patients with another occlusion within 6 months.

Description of Study Intervention: The patients received catheter-directed thrombolysis/thrombectomy to relieve symptoms of femoropopliteal deep vein thrombosis, with or without iliac vein involvement. After completion of DVT recanalization, participants are qualified for enrollment in the study and receive treatment with the investigational drug (a solution containing 80% of 4.0 mg/mL dexamethasone sodium phosphate injection, USP, and 20% of contrast medium with >300 mg unbound iodine per mL). Investigational drug is delivered by Bullfrog Micro-Infusion Device to the adventitia and perivascular tissue around target vein segments. The dosage is delivered in a volume of 0.4 mL (1.28 mg) per cm of target vein length, up to 50 cm, for a total volume of up to 20 mL and a total dose of up to 64 mg dexamethasone.

Study Hypothesis: The hypothesis of the study is that negative outcomes including post-thrombotic syndrome arise from acute, post-thrombotic vein wall inflammation culminating in vein wall scarring, rethrombosis, loss of valve function, loss of venous patency and venous inflammation. The perivascular delivery of dexamethasone may reduce deep vein thrombosis-related inflammation concomitant with removal of thrombus burden, relieving symptoms, reducing the potential for re-thrombosis and vein wall fibrosis, and thereby limiting progression to re-thrombosis and/or post-thrombotic syndrome. The Lead-in Phase initially assesses safety and later with the RCT Phase provide information on treatment effect that is used in designing a pivotal study.

Objectives and Endpoints: The objectives and endpoints for this example are similar in many respects as Example 4.

A primary objective and endpoint of the study was to study the effectiveness of the treatment to limit the rate of clinically relevant loss of primary patency in the fem-pop segment at 6 months following the procedure. Rate of clinically relevant loss of primary patency was measured at 6 months. Clinically relevant loss of primary patency occurs with (a) worsening or non-resolving symptoms of DVT and (b)(i) reintervention of the treated segment or (ii) ultrasound or angiographic detection of rethrombosis of the treated segment causing occlusion in unstented vein or ≥50% narrowing in stented vein. In some cases, the measurement of occlusion may be straightforward with duplex ultrasound techniques. Reducing the rate of clinically relevant (symptomatic) occlusions at 6 months would provide significant clinical benefit.

The safety of the treatment was assessed by its ability to limit the incidence of composite major adverse events (MAE) at 30 days following treatment of an obstruction in the femoropopliteal segment, as measured by one or more of the following events: all-cause death, clinically significant (i.e., symptomatic, confirmed by CT pulmonary angiography) pulmonary embolism, major (BARC 3b or greater) bleeding, target vessel thrombosis confirmed by imaging as assessed by core lab, infection of the treatment or insertion site, or AV fistula at the treatment site. The interventional drug should not cause incremental safety risk beyond the current gold-standard technology. The timepoint is 30 days because the drug delivered should have its principal safety effects in the peri-procedural timeframe, and systemic levels are expected to be negligible within days of the injection.

To assess the effectiveness of the treatment to maintain clinically relevant primary patency of the target vein segment, the rate of clinically relevant primary patency overall and in each segment (CIV, EIV, CFV, PFV, FV, POP) is measured at discharge, 5 weeks, 3, 6, 12, 18 and 24 months. Clinically relevant loss of primary patency occurs with (a) worsening or non-resolving symptoms of DVT and (b)(i) reintervention of the treated segment or (ii) ultrasound or angiographic detection of rethrombosis of the treated segment causing occlusion in unstented vein or ≥50% narrowing in stented vein. Primary patency is a common outcome in venous thrombosis studies Primary patency is defined by an unoccluded target vein segment without re-intervention. Recording those occlusions that are symptomatic may improve understanding of clinical significance.

To assess the effectiveness of the treatment to maintain primary patency of the target vein segment, the rate of primary patency is measured at discharge, 5 weeks, 3, 6, 12, 18 and 24 months. Loss of primary patency occurs with ultrasound or venographic detection of complete occlusion of the treated fem-pop segment or a clinically driven re-intervention of the treated segment.

To assess the effectiveness of the treatment to limit need for clinically driven target vein reintervention, the reintervention rate is assessed at 5 weeks, 3, 6, 12, 18 and 24 months. Reducing reintervention rate may be an important factor to improve a patient's quality of life. The need for reintervention may be tied to worse late-stage outcomes.

To assess the effectiveness of the treatment to limit rate of venous noncompressibility, the rate of venous noncompressibility by ultrasound at discharge, 5 weeks, 6 months, 12 months is measured. In some cases, venous noncompressibility at one month is linked to progression to PTS.

To assess the effectiveness of the treatment to limit residual thrombus as detected by residual vein diameter under compression, the residual thrombus thickness measured by compression ultrasound, in mm, is measured at discharge, 5 weeks, 6 months, 12 months. In some cases, the amount of residual thrombus indicates whether thrombus may be clearing or building back up in the vein.

To assess the effectiveness of the treatment to limit the rate of venous reflux, the rate of venous reflux (time cutoff 1000 ms) as measured by ultrasound is assessed at 6 and 12 months. In some cases, venous reflux indicates dysfunctional valves, which is a key characteristic of PTS

To assess the effectiveness of the treatment to limit the progression to PTS, the PTS rate by Villalta score ≥5 and by PTS rate by VCSS score ≥4 is assessed at 3, 6, 12, 18 and 24 months. Villalta and VCSS scoring systems are commonly used to determine progression and severity of PTS.

To assess the effectiveness of the treatment to limit the overall severity of PTS, the rate of mild PTS by Villalta score 5-9, moderate PTS by Villalta score 10-14, and severe PTS by Villalta score ≥15 is assessed at 3, 6, 12, 18 and 24 months. Also, the rate of mild-to-moderate PTS by VCSS score 4-7, severe PTS by VCSS score ≥8 is assessed at 3, 6, 12, 18 and 24 months. In addition to reducing the rate of progression to PTS, by incrementally reducing the severity of PTS, participants may have better quality of life.

To assess the effectiveness of the treatment to maintain reduced Villalta and VCSS scores versus baseline, the change in Villalta score and VCSS score from baseline to follow-up at 3, 6, 12, 18 and 24 months are assessed. In some cases, evidence of improvement in Villalta or VCSS scores can be useful to demonstrate clinically significant benefit.

To assess the effectiveness of the treatment to limit the leg pain, a change in Likert pain scale (Likert 7-point scale) from baseline to each follow-up at 5 weeks, 3, 6, 12, 18, and 24 months is assessed. In some cases, leg pain may be a key factor in improving a participant's quality of life.

To assess the effectiveness of the treatment to limit the index-leg minimal circumference as measured by a change from baseline, the minimal target leg circumference as measured at 10 cm below the tibial tuberosity of the target leg is taken at 10 day, 5 weeks. In some cases, the index leg circumference helps to determine the degree of edema that a patient is experiencing.

To assess the effectiveness of the treatment to improve quality-of-life outcomes, change in VEINES questionnaire (25-question VEINES-QOL and 10-question VEINES-Sym) scores from baseline to each follow-up at 10 day, 5 week, 3, 6, 12, 18 and 24 months are taken. The VEINES questionnaires are commonly accepted within the field of DVT to establish participant quality of life.

To assess the effectiveness of the treatment, levels of circulating inflammatory biomarkers and change from baseline to follow-ups at 10 days, 5 weeks, 3 months in order to determine systemically detectable changes in inflammation are measured The biomarkers include one or more of: IL-1β, IL-2, IL-6, IL-8, IL-10, IFN-α, IFN-γ, ICAM-1, TNF-α, hsCRP, D-dimer, and fibrinogen.

To assess the safety of the treatment, the rate of serious adverse events in the first 30 days after treatment were observed. In addition, any adverse events (subclassified as major, serious, non-serous, unanticipated, revascularization-procedure-related, device-related and drug-related) were observed to 24 months.

To assess the technical success of the treatment, complete longitudinal and circumferential distribution of drug around the target vein segment are assessed during the procedure by infusion grade and coverage % by angiography. In some cases, the distribution pattern achieved during the adventitial and perivascular drug delivery can be used to potentially correlate drug distribution pattern to positive outcomes.

### Example 6

### In Vivo Human Clinical Study

Provided herein are examples of in vivo human clinical trials using the Bullfrog Micro-Infusion Device for local delivery of dexamethasone. The Bullfrog Micro-Infusion Device was successfully used in the DANCE (Dexamethasone to the Adventitia to Enhance Clinical Efficacy After Femoropopliteal Revascularization)-Pilot study and the DANCE trial in superficial femoral and popliteal arteries.

In the DANCE-Pilot study, 20 subjects were enrolled and treated with the Bullfrog device delivery of dexamethasone sodium phosphate. In the DANCE trial, 283 limbs were enrolled and treated with the Bullfrog device delivery of dexamethasone sodium phosphate. In 3.3% of the subjects enrolled in DANCE, there was no detected contrast medium distribution in the adventitia and perivascular tissues. In one subject in DANCE-Pilot and one subject in DANCE, there was a transient hyperglycemia event reported, which was treated and controlled with insulin therapy. There were no other unexpected adverse device events or suspected unexpected severe adverse reactions reported in the study.

The single-arm DANCE (Dexamethasone to the Adventitia to Enhance Clinical Efficacy After Femoropopliteal Revascularization) trial enrolled 262 subjects (283 limbs) with symptomatic peripheral artery disease (Rutherford category 2 to 4) receiving percutaneous transluminal angioplasty (PTA) (n=124) or atherectomy (ATX) (n=159) in femoropopliteal lesions <15 cm in length. A mixture of dexamethasone/contrast medium (80%/20%) was delivered to the adventitia and perivascular tissues surrounding target lesions in all subjects. Thirty-day assessments included major adverse limb events (MALE) and post-operative death. Twelve-month assessments included primary patency, freedom from clinically driven target lesion revascularization (CD-TLR), Rutherford scoring, and walking impairment questionnaire. At 12 months, primary patency rates in DANCE-ATX and -PTA per-protocol populations were 78.4% (74.8% intent-to-treat [ITT]) and 75.5% (74.3% ITT), respectively. Rates of CD-TLR in DANCE-ATX and -PTA subjects were 10.0% (13.1% ITT) and 11.0% (13.7% ITT), respectively. There were no 30-day MALE+post-operative death events nor 12-month device- or drug-related deaths or MALE. In the primary analysis, both the ATX and PTA DANCE groups (ITT) were superior (P<0.001) to the 52.5% historical performance goal. In the secondary analysis, both the ATX and PTA DANCE groups were noninferior to the 72.3% contemporary performance goal, whether examining the PP (P<0.001 and P<0.004, respectively) or ITT (P<0.002 and P<0.005, respectively) population.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### REFERENCES

Rondina M T, Lam U T, Pendleton R C, Kraiss L W, Wanner N, Zimmerman G A, Hoffman J M, Hanrahan C, Boucher K, Christian P E, Butterfield R I, Morton K A. (18)F-FDG PET in the evaluation of acuity of deep vein thrombosis. Clin Nucl Med. 2012 December; 37(12):1139-45. doi: 10.1097/RLU.0b013e3182638934. PMID: 23154470; PMCID: PMC3564643.

Mosevoll K A, Johansen S, Wendelbo Ø, Nepstad I, Bruserud Ø, Reikvam H. Cytokines, Adhesion Molecules, and Matrix Metalloproteases as Predisposing, Diagnostic, and Prognostic Factors in Venous Thrombosis. Frontiers in medicine 2018;5:147.

Roumen-Klappe E M, Janssen M C, Van Rossum J, Holewijn S, Van Bokhoven M M, Kaasjager K, Wollersheim H, Den Heijer M. Inflammation in deep vein thrombosis and the development of post-thrombotic syndrome: a prospective study. J Thromb Haemost. 2009 April; 7(4):582-7. doi: 10.1111/j.1538-7836.2009.03286.x. Epub 2009 Jan. 19. PMID: 19175493.),

1. A method of reducing progression to post-thrombotic syndrome (PTS) in a subject, the method comprising:
(a) identifying a vein in the subject affected by deep vein thrombosis (DVT) currently or previously and/or is at risk for progressing to PTS;
(b) advancing a therapeutic delivering catheter within a lumen of the vein affected by DVT to or near a thrombosed segment of the vein; and
(c) delivering a therapeutic composition into a perivascular tissue at or near the thrombosed segment using the therapeutic delivering catheter,
wherein the therapeutic composition comprises an anti-inflammatory agent and a therapeutic dosage of the anti-inflammatory agent ranges from about 0.1 mg per cm of the thrombosed segment to about 10 mg per cm of the thrombosed segment.

2. The method of claim 1, wherein the anti-inflammatory agent comprises a glucocorticoid.

3. The method of claim 2, wherein the glucocorticoid comprises dexamethasone.

4. The method of claim 3, wherein the vein affected by DVT comprises a plurality of thrombotic segments.

5. The method of claim 4, wherein the therapeutic composition is delivered to the plurality of thrombosed segments.

6. The method of claim 1, wherein the vein affected by DVT has undergone a catheter-directed thrombolysis or thrombectomy (CDT) previously.

7. The method of claim 1, wherein the vein affected by DVT has undergone an endovascular procedure previously, wherein the endovascular procedures comprises one or more of venous valve repair, venous bypass, and venous stents.

8. The method of claim 1, wherein a total dosage of the anti-inflammatory agent delivered into the vein affected by DVT ranges between about 1 mg and about 100 mg.

9. The method of claim 1, wherein a therapeutic concentration of the anti-inflammatory agent delivered into the vein affected by DVT ranges between about 0.1 mg/ml to about 10 mg/ml.

10. The method of claim 9, wherein a volume of the anti-inflammatory agent delivered into the vein affected by DVT ranges between about 0.01 ml per cm of the thrombosed vein to about 100 ml per cm of the thrombosed vein.

11.-18. (canceled)

19. The method of claim 1, wherein a level of one or more inflammatory biomarkers decreases after the delivery of a therapeutic composition into a perivascular tissue at or near the thrombosed segment.

20. The method of claim 19, wherein the one or more inflammatory biomarkers comprises one or more of IL-1β, IL-2, IL-6, IL-8, IL-10, IFN-α, IFN-γ, ICAM-1, TNF-α, CRP, D-dimer, fibrinogen, MCP-1, IL-1Ra, IL-1α, MMP-1, MMP-2, MMP-8, MMP-9, TIMP, ICAM-1, VCAM-1, and soluble P-selectin.

21. The method of claim 19, wherein the level of one or more inflammatory biomarkers is measured from a sample from whole blood, plasma, serum, or perivascular tissue.

22. The method of claim 1, wherein a level of one or more anti-inflammatory biomarkers increases after the delivery of a therapeutic composition into a perivascular tissue at or near the thrombosed segment.

23. (canceled)

24. The method of claim 1, wherein the reduction in progression to PTS is assessed by maintenance or an increase in patency of the thrombosed segment.

25. The method of claim 24, wherein the maintenance or the increase in patency lasts for at least 5 weeks, 3 months, 6 months, 12 months, 18 months, or 24 months.

26. The method of claim 1, wherein the reduction in progression to PTS is assessed by a decrease or a lack of increase in rethrombosis in the thrombosed segment.

27. The method of claim 26, the decrease or the lack of increase in rethrombosis lasts for at least 5 weeks, 3 months, 6 months, 12 months, 18 months, or 24 months.

28. (canceled)

29. The method of claim 1, wherein the reduction in progression to PTS is assessed by a decrease or a lack of increase in venous reflux.

30. The method of claim 29, wherein the decrease or the lack of increase in venous reflux lasts for at least 5 weeks, 3 months, 6 months, 12 months, 18 months, or 24 months.

31. (canceled)

32. The method of claim 1, wherein the reduction in progression to PTS is assessed by a decrease or a lack of increase in fibrosis and stiffness of wall and valve of the vein affected by DVT.

33. (canceled)

34. The method of claim 1, wherein the reduction in progression to PTS is assessed by a decrease or a lack of increase in a symptom of PTS, wherein the symptom of PTS comprises one or more of pain, cramps, heaviness, pruritus, paresthesia, edema, skin induration, hyperpigmentation, venous ectasia, redness, and pain during calf compression.

35. The method of claim 1, wherein the reduction in progression to PTS is assessed by a decrease or a lack of increase in a Villalta score or a VCSS score.

36. The method of claim 1, wherein the vein affected by DVT currently or previously and/or is at risk for progressing to PTS is identified by fluordeoxyglucose-positron emission tomography (FDG-PET).

37. The method of claim 1, wherein the reduction in progression to PTS is assessed by FDG-PET scanning of the perivascular tissue.

38. (canceled)

39. (canceled)

40. The method of claim 37, wherein an increase in a residual local metabolic activity detected by FDG-PET indicates progression to PTS.

41. (canceled)

42. The method of claim 1, wherein the therapeutic composition comprises one or more component for extended release, sustained release, or controlled release.

43.-84. (canceled)

85. A system for use in reducing progression to post-thrombotic syndrome (PTS) in a subject according to the method of claim 1, the system comprising:
a therapeutic composition comprising an anti-inflammatory agent;
a catheter configured to be placed within a vein affected by deep vein thrombosis (DVT) in the subject;
an expandable element at a distal end of the catheter, wherein the expandable element is inflatable from an involuted contracted configuration; and
an injection needle coupled to the expandable element,
wherein expanding the expandable element advances the injection needle in a direction transverse to a longitudinal axis of the catheter to puncture wall of the vein at or near a thrombosed segment of the vein, and
wherein, when the needle has punctured the wall of the vein, the needle delivers an amount of the therapeutic composition to a perivascular tissue at or near a thrombosed segment of the vein, the amount being therapeutic to reducing progression to PTS.

86. The system of claim 85, wherein the expandable element is expandable to a circumference to fill a lumen of the vein, wherein the circumference is larger than 2 mm.
